(19) United States
(12) Patent Application Publication
(10) Pub. No.: US 2013/0102785 A1
(43) Pub. Date: Apr. 25, 2013

(54) METHOD OF MAKING COUPLED HETEROARYL COMPOUNDS VIA REARRANGEMENT OF HALOGENATED HETEROAROMATICS FOLLOWED BY OXIDATIVE COUPLING

(71) Applicants: Seth Marder, Atlanta, GA (US); Yulia Alexandrovna Getmanenko, Atlanta, GA (US)

(72) Inventors: Seth Marder, Atlanta, GA (US); Yulia Alexandrovna Getmanenko, Atlanta, GA (US)

(73) Assignee: Georgia Tech Research Corporation, Atlanta, GA (US)

(21) Appl. No.: 13/578,238

(22) PCT Filed: Feb. 9, 2011
(86) PCT No.: PCT/EP2011/051913

§ 371 Date: Jan. 3, 2013

Related U.S. Application Data
(60) Provisional application No. 61/303,163, filed on Feb. 10, 2010.

(51) Int. Cl. C07F 7/08; C07D 495/22; C07D 495/04; C07D 409/04; C07D 401/04

(52) U.S. Cl. CPC C07F 7/083; C07D 409/04; C07D 401/04; C07F 7/0827; C07D 495/04; C07D 495/22
USPC 546/259; 549/4; 540/1; 549/59; 548/110; 549/44

(57) ABSTRACT

The inventions disclosed and described herein relate to new and efficient generic methods for making a wide variety of compounds having Formulas (I) and (II) as shown below (Formulas (I) and (II)) wherein HAr is an optionally substituted five or six membered heteroaryl ring, and Hal is a halogen, and Y is a bridging radical, such as S, Se, NR⁵C(O), C(O)C(O), Si(R⁵)₂, SO, SO₂, PR⁵, BR⁵, C(R⁵)₂ or P(O)R⁵. The synthetic methods employ a “Base-Catalyzed Halogen Dance” reaction to prepare a metallated compound comprising a five or six membered heteroaryl ring comprising a halogen atom, and then oxidatively coupling the reactive intermediate compound. The compounds of Formula (II) and/or oligomer or polymers comprising repeat units having Formula (II) can be useful for making semi-conducting materials, and/or electronic devices comprising those materials.

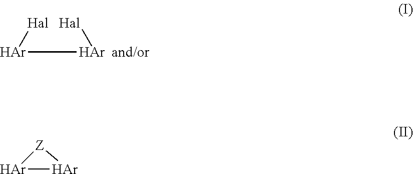

### RELATED APPLICATIONS

This application claims the priority of U.S. Provisional Application No. 61,303,163 filed 10 Feb. 2010, the whole content of this application being incorporated herein by reference for all purposes.

### STATEMENT OF GOVERNMENT LICENSE RIGHTS

The inventors received partial funding support through the STC Program of the National Science Foundation under Agreement Number DMR-0120967 and the Office of Naval Research through a MURI program, Contract Award Number 68A-1060806. The Federal Government has certain rights in this invention.

### TECHNICAL FIELD OF THE INVENTION

The various inventions disclosed, described, and/or claimed herein relate to the field of synthesis of organic compounds comprising coupled heteroaryl rings, and compounds produced by the methods of the invention, many of which are useful for the preparation of monomeric, oligomeric, or polymeric organic compounds useful in making organic electronic devices, such as transistors, solar cells, and light emitting diodes.

### BACKGROUND OF THE INVENTION

In recent years there has been a good deal of interest in the art in creating new semiconducting organic materials (monomeric, oligomeric, or polymeric) that comprise conjugated aromatic and/or heteroaromatics rings, and are capable of conducting electrical charge carriers (holes and/or electrons) for use in making various electronic devices, such as for example transistors, solar cells, and light emitting diodes.

While existing semiconductor technologies based on inorganics such as silicon, germanium, etc are highly developed, fabrication of those devices is expensive and the resulting devices are rigid and fragile. The development of new and physically flexible and solution processable organic semiconducting materials could allow the inexpensive fabrication of electronic devices and light emitting displays on inexpensive flexible materials such as plastics, organic coatings, etc. Accordingly, there remains a need in the art for new and improved organic semiconductors, that can provide improved processability, performance, cost, and stability in use in organic electronic devices.

Some progress has already been made in synthesizing such new organic semiconducting materials, including semiconducting organic polymers or copolymers, but many of those organic semiconducting materials comprise highly specifically designed and substituted aryl or heteroaryl subunits, including subunits comprising multiple conjugated and/or fused ring subunits. Unfortunately, known synthetic methods employed to synthesize monomeric aryl and heteroaryl polymer precursors of the organic materials are still often exotic and expensive, and the ultimate performance of the final organic semiconductors could still use significant improvements. Accordingly, there remains a need in the art for improved methods for making polymerizable monomeric or oligomeric precursors of new and improved semiconducting organic materials.

Aryl and heteroaryl halides, especially bromide and iodides, are well known as polymerizable precursors of such semiconducting small molecules, oligomers, polymers and copolymers, and are also well known to be convertible to aryl or heteroaryl boronic ester or trialkyl tin derivatives that are also polymerizable or can be reacted to make small molecules and oligomers (typically in the presence of transition metal polymerization catalysis such as palladium or nickel complexes). Synthetic methods for making many such aryl or heteroaryl halide compounds are known, but the synthesis of particular desirable isomers of many aryl or heteroaryl halides remain difficult or expensive.

It is to that end that the various embodiments of the various inventions described below, which relate to new methods for producing polymerizable monomers or oligomers, or the required synthetic precursors, are directed.

It is known in the art that aryl or heteroaryl halides can sometimes be isomerized to move the halogen to a different position on the aryl or heteroaryl ring if they are treated with very strong bases, such as for example organo-lithium or organo-magnesium reagents, or lithium dialkylamides. This base catalyzed rearrangement of aryl and heteroaryl halides, called the “Base-Catalyzed Halogen Dance” (“BCHD”) rearrangement (see, for example Schnurich et al, Chem. Soc. Rev., 2007, 36, 1046-1057, and de Souza, Curr. Org. Chem. 2007, 11, 637-646) both hereby incorporated by reference for their teachings regarding the methodology of the Halogen Dance reaction and its known synthetic applications) is, although not wishing to be bound by theory, believed in the art to occur via deprotonation of a relatively acidic hydrogen on the ring of a halogenated aryl or heteroaryl starting material by a strongly basic organometallic reagent (typically organo lithium, organomagnesium, or lithium dialkylamide compounds), to form a metallated (often lithiated) form of the starting halo aryl or heteroaryl. The metallated halo aryl or heteroaryl can then undergo a series of metal-halogen exchange reactions that can result in migration of the original halogen substituent to a more thermodynamically stable position on the original aryl or heteroaryl ring (as envisioned in the conceptual schematic diagram below, where Het is a ring heteroatom, Hal is a halogen, and M is often Li or Mg).

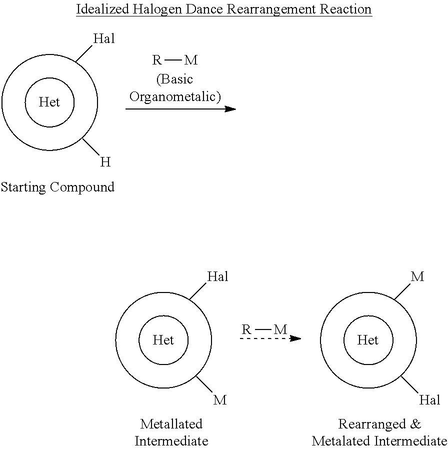

The rearranged and metallated halogenated intermediate heteroaryl compounds formed via the Halogen Dance rearrangement have then been further utilized in a variety of ways in the prior art, especially by reactions with electrophiles, but those prior uses are believed to be significantly different in kind than the uses of those halogenated and metallated heteroaryl intermediates for oxidative couplings described and claimed hereinbelow.

It is known in the art that the rings of some metallated (typically lithiated) aryl or heteroaryl compounds can be oxidatively coupled with certain oxidizing agents such as copper salts or thionyl chloride, as schematically indicated in the idealized drawing below (see for example, Gronowitz, S. Acta Chem. Scand. 15, 1393-1395 (1961); Whitesides et al, J. Amer. Chem. Soc. 89(20) 5302-5303 (1967); Surry et al, Angew Chem. Int. Ed., 44, 1870-1873 (2005), and Oae et al, Phosphorus, Sulfur, and Silicon, Vol. 103, 101-110 (1995), hereby incorporated by reference for their various teachings regarding relevant oxidative coupling reactions).

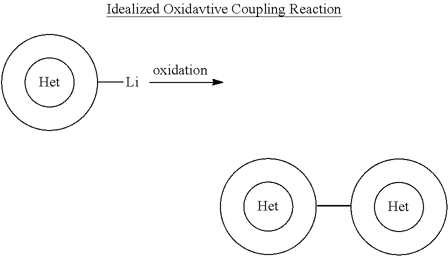

Lastly, it has long been known in the art, such as for example as recently disclosed in PCT Publication WO 2009/115413 (hereby incorporated by reference herein) that certain bishalogenated bisthiophene compounds could be coupled with various regents to form a class of fused ring bisthiophene heteroaryls as indicated in the reaction scheme below:

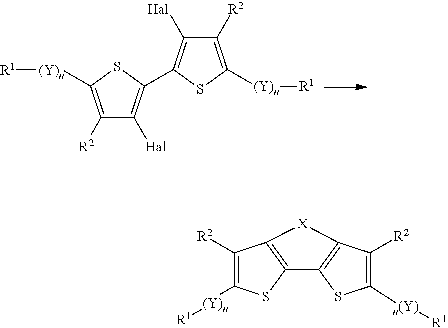

W herein Hal stands for hydrogen or halogen, especially Br, R¹ is hydrogen or a substituent, n ranges from 0 to 6, preferably being 0; Y, if present, is substituted or unsubstituted phenylene, thiene, 1,2-ethylene, or is 1,2-ethinylene; R² is hydrogen or certain aryls and alkyls, and X is certain bridging groups. WO 2009/115413 taught that its compounds and/or certain copolymers derived therefrom could be useful as semiconductors for making electronic devices. WO 2009/115413 did not however teach or suggest that a combination of the halogen dance reaction and an oxidative coupling reaction could be used to prepare its bishalogenated bisthiophene starting materials, or that fused ring heterocycle that do not comprise at least two thiophene rings could be prepared by the methods disclosed.

The various inventions described hereinbelow relate to a sequence of reactions that can be used to conveniently and economically prepare a very wide variety of both known and new dihalo-aryl and/or heteroaryl intermediates, which serve as precursors for the preparation of reactive small molecules that can be used as precursors for the synthesis of new small molecules, oligomers, polymers, and co-polymers that can be useful for making organic electronic devices.

### SUMMARY OF THE INVENTION

The various inventions and/or their embodiments disclosed herein relate to new methods for making heteroaryl compounds having at least two coupled heteroaryl rings and two halogens that employ a sequence of reactions that involve the use of the Base-Catalyzed Halogen Dance (BCHD) reaction to prepare optionally substituted heteroaryl intermediates that are then oxidatively coupled, to prepare a very wide variety of heteroaryl small molecule, oligomer, polymer, and co-polymer compounds having at least two coupled heteroaryl rings.

In many embodiments, the inventions relate to various methods for synthesizing a bishalo-bisheteroaryl compound comprising the compound of Formula (I)

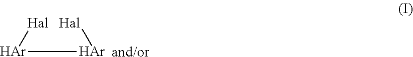

wherein HAr is an optionally substituted five or six membered heteroaryl ring comprising at least one ring carbon atom and at least one ring heteroatom, and Hal is a halogen. While there are many embodiments of the disclosed methods for making the compounds of Formula (I), in many of those embodiments the steps of the method comprise at least:
a. providing an optionally substituted precursor compound comprising a halo-heteroaryl ring having an Hal substituent at a first position on the HAr ring;
b. treating the precursor compound with a strongly basic compound to induce the isomerization of the precursor compound to produce an intermediate compound wherein the Hal atom is bound to a different position on the HAr ring;
c. treating the intermediate compound with an oxidizing agent so as to form a carbon-carbon bond between two intermediate compounds and thereby form the bishalo-bisheteroaryl compound.

Both some known bishalo-bisheteroaryl compounds and also a variety of new and non-obvious bishalo-bisheteroaryl compounds can be readily and efficiently prepared via the “Base-Catalyzed Halogen Dance/Oxidative Coupling” reaction sequences disclosed, described, and/or claimed herein. Many of the bishalo-bisheteroaryl compounds of the invention comprise two coupled heteroaryl radicals, and have the structure shown in Formula (Ia):

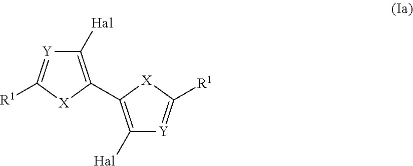

wherein
a. R¹ can be hydrogen, a halide, or a C₁-C₃₀ organic radical, such as for example optionally substituted alkyl, alkynyl, aryl, and heteroaryl radicals, or —Sn(R²)₃, —Si(R²)₃, or —B(—OR²¹)₂ radicals, wherein each R² is an independently selected alkyl or aryl, and each R²¹ is an independently selected alkyl or aryl, or the R²¹ groups together form an optionally substituted alkylene group bridging the oxygen atoms;
b. X can be O, S, Se, or NR³ wherein R³ is a C₁-C₁₈ alkyl, perfluoroalkyl, aryl, or heteroaryl; and
c. Y can be CH, CR⁴, or N, wherein R⁴ is a C₁-C₁₈ alkyl, aryl, or heteroaryl.

Moreover, many of both the novel and known the bishalo-bisheteroaryl compounds produced by the BCHD/oxidative coupling methods can be easily further functionalized and/or elaborated to produce a wide variety of known or new downstream compounds, oligomers, or polymers that are useful for many purposes.

Among the compounds that can be prepared from the bishalo-bisheteroaryl compounds of Formula (I) produced by the BCHD/oxidative coupling methods are a wide variety of fused tricyclic compounds of Formula (II), as shown below:

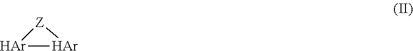

wherein
a. HAr can be any of the optionally substituted heteroaryl rings disclosed elsewhere herein, and
b. Z is a bridging group, such as for example S, Se, NR⁵, C(O), C(O)C(O), Si(R⁵)₂, SO, SO₂, PR⁵, P(O)R⁵, BR⁵, or C(R⁵)₂, wherein R⁵ is a C₁-C₅₀ organic radical.

Many fused tricyclic compounds of Formula (II) can be prepared by
a. optionally treating a bishalo-bisheteroaryl compound with an organometallic compound to exchange a metal for a Hal substituent, and form a bismetallo-bisheteroaryl compound, and
b. reacting the bismetallo-bisheteroaryl compound with a suitable electrophile, or reacting the bishalo-bisheteroaryl compound or bismetallo-bisheteroaryl compound with a nucleophile, to introduce the Z group, or a precursor thereof suitable for forming the fused tricyclic compound.

Examples of such fused tricyclic compounds include but are not limited to compounds of Formula (IIa) shown below:

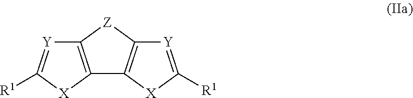

wherein
a. R¹ can be hydrogen, a halide, or a C₁-C₃₀ organic radical selected from optionally substituted alkyl, alkynyl, aryl, and heteroaryl, or —Sn(R²)₃, —Si(R²)₃, Si(OR²)₃, or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b. X can be O, S, Se, or NR³ wherein R³ is a C₁-C₁₈ alkyl, perfluoroalkyl, aryl, or heteroaryl; and
c. Y can be CH, CR⁴, or N, wherein R⁴ is a C₁-C₁₈ alkyl, aryl, or heteroaryl; and
d. Z can be S, Se, NR⁵, C(O), C(O)C(O), Si(R⁵)₂, SO, SO₂, PR⁵, P(O)R⁵, BR⁵, or C(R⁵)₂, wherein R⁵ is a C₁-C₅₀ organic radical selected from optionally substituted alkyl, perfluoroalkyl, aryl, and heteroaryl.

In many embodiments of the compounds of Formula (IIa), R¹ can be an optionally substituted aryl, or heteroaryl radical. For example, in many embodiments of the bishalo-bisheteroaryl compounds of Formula (I) or the fused tricyclic compounds of Formula (II), R¹ can be a relatively electron rich radical having one of the formulas shown below:

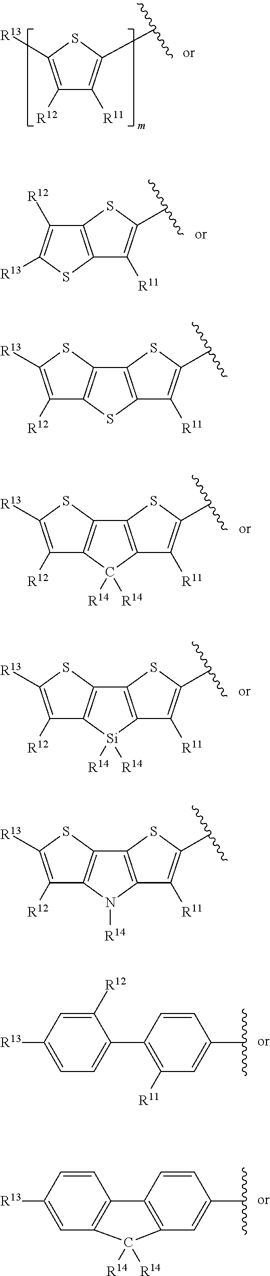

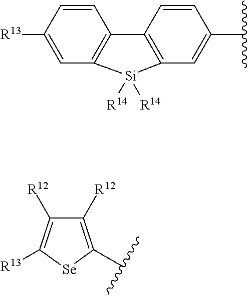

wherein R¹¹-R¹⁴ are defined elsewhere hereinbelow.

In other embodiments of the bishalo-bisheteroaryl compounds of Formula (I) or the fused tricyclic compounds of Formula (IIa), R¹ can be a relatively electron poor heteroaryl radical, such as for example one of the formulas shown below:

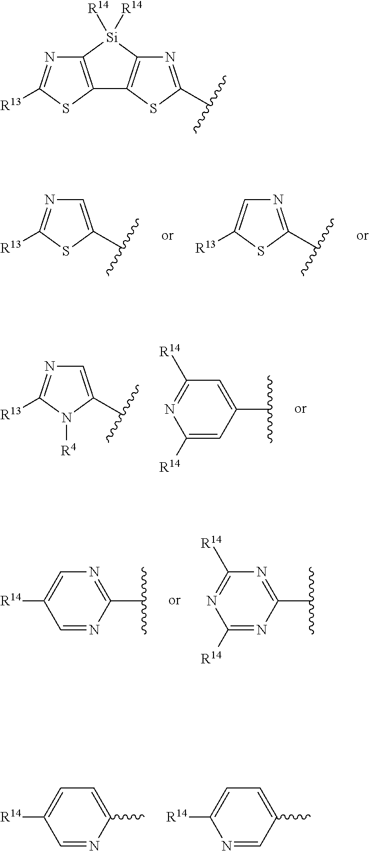

The various genera and subgenera of compounds of Formula (II) or (IIa), prepared by the methods of the invention, can be readily further functionalized and/or elaborated to produce a wide variety of known and new downstream compounds, oligomers, or polymers that are useful for many purposes, including for the preparation of compounds and compositions for making electronic devices, such as transistors, solar cells, light emitting diodes, and the like.

Further detailed description of preferred embodiments of the various inventions broadly outlined above will be provided below in the Detailed Description section provided below.

### BRIEF DESCRIPTION OF THE FIGURES

FIG. 1 discloses the aromatic region of ¹H NMR spectra (400 MHz, CDCl₃) of (a) starting 2-(5-trimethylsilyl-3-n-hexyl-thiophen-2-yl)-5-bromothiazole and (b) its BCHD reaction product, 2-(5-trimethylsilyl-3-n-hexyl-thiophen-2-yl)-4-bromothiazole (signal at 2907.23 for Hz (a) and 7.27 ppm for (b) are residual CHCl₃). See Example 7.

### DETAILED DESCRIPTION OF THE INVENTION

The various inventions and/or their embodiments disclosed herein relate to new methods for making heteroaryl compounds of Formula (I) having at least two coupled heteroaryl rings and two halogens, which employ a sequence of reactions that involve the use of the Base-Catalyzed Halogen Dance (BCHD) reaction to prepare optionally substituted heteroaryl intermediates (in-situ), which are then oxidatively coupled, to prepare a very wide variety of bishalo-bisheteroaryl compounds having at least two coupled heteroaryl rings. Many of the bishalo-bisheteroaryl compounds can then be used to prepare a wide variety of fused tricyclic compounds of Formula (II) as shown above and below, and oligomers, polymers, and copolymers derived therefrom. Such compounds can be used to prepare chemical compositions for making electronic devices, such as transistors, solar cells, light emitting diodes, and the like. In addition they can be used to make various light absorbing materials that could have applications in the fields of sensing, nonlinear optics, optical limiting, as well.

Nevertheless, before describing the many possible embodiments of the inventions described herein, it is desirable to set forth certain relevant definitions.

### DEFINITIONS

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be individually selected from a group consisting of two or more of the recited elements or components.

In addition, where the use of the term “about” is presented before a quantitative value, the present teachings also include the specific quantitative value itself, unless specifically stated otherwise. In some embodiments, the term “about” can refer to a +−10% variation from the nominal value stated.

It should be understood that the order of steps or order for performing certain actions can be immaterial so long as the methods disclosed herein remain operable. Moreover, two or more steps or actions may also be conducted simultaneously, so long as the methods disclosed herein remain operable.

As used herein, a “polymer” or “polymeric compound” refers to a molecule (e.g., a macromolecule) including a plurality of one or more repeating units connected by covalent chemical bonds. A polymer can be represented by the general formula:

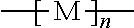

wherein M is the repeating unit or monomer, and n is the number of M's in the polymer. For example, if n is 3, the polymer shown above is understood to be:
M-M-M.

The polymer or polymeric compound can have only one type of repeating unit as well as two or more types of different repeating units. In the former case, the polymer can be referred to as a homopolymer. In the latter case, the term “copolymer” or “copolymeric compound” can be used instead, especially when the polymer includes chemically significantly different repeating units. The polymer or polymeric compound can be linear or branched. Unless specified otherwise, the assembly of the repeating units in the copolymer can be head-to-tail, head-to-head, or tail-to-tail. In addition, unless specified otherwise, the copolymer can be a random copolymer, an alternating copolymer, or a block copolymer.

As used herein, “halo” or “halogen” refers to fluoro, chloro, bromo, and iodo.

As used herein, “oxo” refers to a double-bonded oxygen (i.e., ═O).

As used herein, “alkyl” refers to a straight-chain or branched saturated hydrocarbon group. Examples of alkyl groups include methyl (Me), ethyl (Et), propyl (e.g., -propyl and /iso-propyl), butyl (e.g., n-butyl, iso-butyl, sec-butyl, tert-butyl), pentyl groups (e.g., n-pentyl, neopentyl), hexyl groups, and the like. In various embodiments, an alkyl group can have 1 to 40 carbon atoms (i.e., C₁₋₄₀ alkyl group), or, 1-20 carbon atoms (i.e., C₁₋₂₀ alkyl group). In some embodiments, an alkyl group can have 1 to 6 carbon atoms, and can be referred to as a “lower alkyl group”. Examples of lower alkyl groups include methyl, ethyl, propyl (e.g., n-propyl and iso-propyl), and butyl groups (e.g., n-butyl, sec-butyl, tert-butyl). In some embodiments, alkyl groups can be substituted as described herein. An alkyl group is generally not substituted with another alkyl group, an alkenyl group, or an alkynyl group.

As used herein, “haloalkyl” refers to an alkyl group having one or more halogen substituents. At various embodiments, a haloalkyl group can have 1 to 40 carbon atoms (i.e., C₁₋₄₀ haloalkyl group), for example, 1 to 20 carbon atoms (i.e., C₁₋₂₀ haloalkyl group). Examples of haloalkyl groups include CF₃, C₂F₅, CHF₂, CH₂F, CCl₃, CHCl₂, CH₂Cl, C₂Cl₅, and the like. Perhaloalkyl groups, i.e., alkyl groups where all of the hydrogen atoms are replaced with halogen atoms (e.g., CF₃ and C₂F₅), are included within the definition of “haloalkyl.”

As used herein, “alkoxy” refers to —O-alkyl group. Examples of alkoxy groups include, but are not limited to, methoxy, ethoxy, propoxy (e.g., n-propoxy and iso-propoxy), t-butoxy, pentoxy, hexoxy groups, and the like. The alkyl group in the —O-alkyl group can be substituted as described herein.

As used herein, “cycloalkyl” refers to a non-aromatic carbocyclic group including cyclized alkyl, alkenyl, and alkynyl groups. In various embodiments, a cycloalkyl group can have 3 to 22 carbon atoms, for example, 3 to 20 carbon atoms (e.g., C₃₋₁₄ cycloalkyl group). A cycloalkyl group can be monocyclic (e.g., cyclohexyl) or polycyclic (e.g., containing fused, bridged, and/or spiro ring systems), where the carbon atoms are located inside or outside of the ring system. Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. In some embodiments, cycloalkyl groups can be substituted as described herein.

As used herein, “heteroatom” refers to an atom of any element other than carbon or hydrogen and includes, for example, nitrogen, oxygen, silicon, sulfur, phosphorus, and selenium.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or cycloheteroalkyl rings. An aryl group can have 6 to 24 carbon atoms in its ring system (e.g., C₆₋₂₀ aryl group), which can include multiple fused rings. In some embodiments, a polycyclic aryl group can have 8 to 24 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system).

As used herein, “heteroaryl” refers to an aromatic ring system containing at least one ring heteroatom selected from oxygen (O), nitrogen (N), sulfur (S), silicon (Si), and selenium (Se). The heteroaryl rings typically comprise a five or six membered aromatic ring, which may however be bonded to additional rings, so as to form a polycyclic ring system where at least one of the rings present in the ring system is aromatic and contains at least one ring heteroatom. Polycyclic heteroaryl groups include those having two or more heteroaryl rings fused together, as well as those having at least one monocyclic heteroaryl ring fused to one or more aromatic carbocyclic rings, non-aromatic carbocyclic rings, and/or non-aromatic cycloheteroalkyl rings. A heteroaryl group, as a whole, can have, for example, 5 to 24 ring atoms and contain 1-5 ring heteroatoms (i.e., 5-20 membered heteroaryl group). The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5- or 6-membered monocyclic and 5-6 bicyclic ring systems shown below:

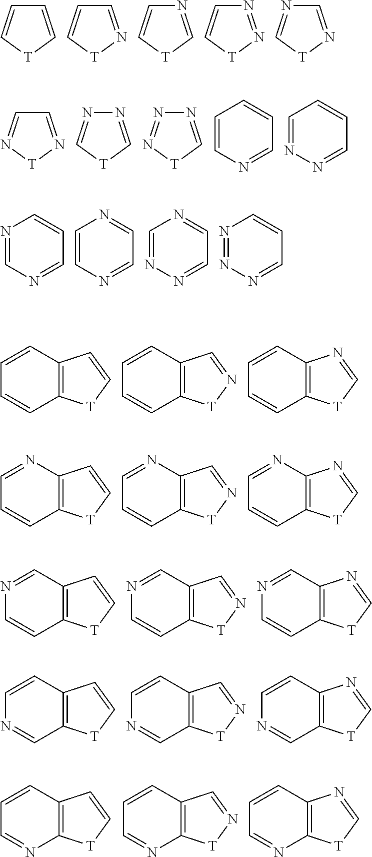

where T is O, S, NH, N-alkyl, N-aryl, N-(arylalkyl) (e.g., N-benzyl), SiH₂, SiH(alkyl), Si(alkyl)₂, SiH(arylalkyl), Si(arylalkyl)₂, or Si(alkyl)(arylalkyl). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzotbiazolyl, benzisothiazolyl, benzisoxazolyl, benzoxadiazolyl, benzoxazolyl, cinnolinyl, 1H-indazolyl, 2H-indazolyl, indolizinyl, isobenzofuryl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In some embodiments, heteroaryl groups can be substituted as described herein.

As used herein, a “p-type semiconductor material” or a “p-type semiconductor” refers to a semiconductor material having holes as the majority current carriers. In some embodiments, when a p-type semiconductor material is deposited on a substrate, it can provide a hole mobility in excess of about 10⁻⁵ cm²/Vs. In the case of field-effect devices, a p-type semiconductor can also exhibit a current on/off ratio of greater than about 10, or preferably greater than about 10⁵.

As used herein, an “n-type semiconductor material” or an “n-type semiconductor” refers to a semiconductor material having electrons as the majority current carriers. In some embodiments, when an n-type semiconductor material is deposited on a substrate, it can provide an electron mobility in excess of about 10⁻⁵ cm²/Vs. In the case of field-effect devices, an n-type semiconductor can also exhibit a current on/off ratio of greater than about 10, or preferably greater than about 10⁵.

As used herein, “solution-processable” refers to compounds (e.g., polymers), materials, or compositions that can be used in various solution-phase processes including spin-coating, printing (e.g., inkjet printing, screen printing, pad printing, offset printing, gravure printing, flexographic printing, lithographic printing, mass-printing and the like), spray coating, electrospray coating, drop casting, dip coating, and blade coating.

### Methods for Synthesizing Bishalo-Bisheteroaryl Compounds

The various inventions and/or their embodiments disclosed herein relate to new methods for making heteroaryl compounds having at least two coupled heteroaryl rings and two halogens, via a sequence of reactions that involve the use of the Base-Catalyzed Halogen Dance (BCHD) reaction to prepare optionally substituted heteroaryl intermediates that have a halogen (especially Br or I) bonded to the heteroaryl ring, and also typically have a main group metal (such as Li or Mg) bonded to the ring. The highly reactive metallated and halogenated heteroaryl rings produced by a BCHD reaction are then oxidatively coupled, to prepare a very wide variety of heteroaryl compounds having at least two coupled heteroaryl rings and two halogens.

In many embodiments, the inventions relate to various methods for synthesizing a bishalo-bisheteroaryl compound of Formula (I)

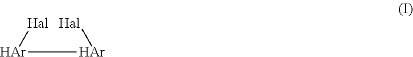

wherein HAr is an optionally substituted five or six membered heteroaryl ring, which comprises at least one ring carbon atom and at least one ring heteroatom, and Hal is a halogen, especially Br or I. In many embodiments of the methods, HAr is a five membered heteroaryl ring that may optionally be substituted with additional organic or inorganic substituent groups, including additional aryl or heteroaryl rings. In various embodiments, the HAr ring and its optional substituents together comprise between 1 to 50, or 2 to 40, or 3 to 30 carbon atoms.

The method for synthesizing the compounds of Formula (I) comprise at least the following steps:
a. providing an optionally substituted precursor compound comprising a halo-heteroaryl ring having an Hal substituent at a first position on the HAr ring;
b. treating the precursor compound with a strongly basic compound to induce the isomerization of the precursor compound to produce an intermediate compound wherein the Hal atom is bound to a different position on the HAr ring;
c. treating the intermediate halo-heteroaryl compound with an oxidizing agent so as to form a carbon-carbon bond between two intermediate halo-heteroaryl compounds and thereby form the bishalo-bisheteroaryl compound.

The optionally substituted precursor compound comprises at least one halo-heteroaryl ring having the Hal substituent (typically Br or I) at a first position on the HAr ring, but may also have other organic or inorganic ring substituents, including additional halides, and other aryl or heteroaryl ring at other positions of the HAr heteroaryl ring. A preferred group of ring substituents for HAr include aryl or heteroaryl rings, fluoride, cyano, alkyl, alkynyl, alkoxy, perfluoroalkyl, and perfluoroalkoxy groups that can significantly modulate the electronic properties of the HAr ring, modify the solubilities or other physical properties, and/or are substantially chemically stable after oxidation by holes or reduction by the electrons used as current carriers in electronic devices. The ring substituents for HAr can also be certain functional groups such as trialkyltin, trialkylsilicon, trialkoxysilicon, or organoborate ester groups that are well known as useful for subsequent cross-coupling with or polymerization of the compounds of Formula (I) or (II).

In many embodiments, the precursor compound for the methods of synthesis is also the precursor for the HAr rings, and have the structure

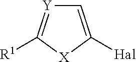

wherein
a. R¹ is a halide, or an optionally substituted organic radical;
b. X is O, S, Se, or NR³ wherein R³ is a C₁-C₁₈ alkyl, perfluoroalkyl, aryl, or heteroaryl; and
c. Y is CH, CR⁴, or N, wherein R⁴ is a C₁-C₁₈ alkyl, aryl, or heteroaryl.

Preferred R¹ organic radicals, which can be attached to the five-membered heteroaryl ring at the position indicated in the drawing either before or after the halogen dance/oxidative coupling reaction steps, can be an C₁-C₃₀ organic radical, such as for example an alkyl, alkynyl, aryl, heteroaryl, —Sn(R²)₃ (triorganotin), —Si(R²)₃ (triorganosilyl), Si(OR²)₃ (trialkoxysilyl) or —B(—OR²¹)₂ (organoborate ester) group wherein each R² is an independently selected alkyl or aryl, and each R²¹ is an independently selected alkyl or aryl, or the R²¹ groups together form an optionally substituted alkylene group bridging the oxygen atoms.

Preferred triorganotin radicals include trialkyltin radicals, especially tributyltin and trimethyltin radicals, which are well known for their use in palladium-catalyzed Stille coupling and/or polymerization reactions with organic halides, especially aryl or heteroaryl bromides or iodides. Preferred triorganosilyl radicals include trialkylsilyl radicals, especially trimethylsilyl (TMS) radicals or triisopropylsilyl (TIPS) radicals, which can be easily converted to halides such as bromides and iodides, or directly react in the Hiyama coupling (for activated TMS groups). Preferred trialkoxysilyl radicals include trimethoxysilyl, or triethoxylsilyl, or tripropoxysilyl radicals. Preferred organoborate ester groups comprise alkyl groups at R², or are pinnacol borate radicals (ie. 4,4,5,5-tetramethyl-1,3,2-dioxaborolane groups having the structure shown below, which are well known for their reactivity in palladium catalyzed Suzuki coupling reactions with other organic halides, especially aryl or heteroaryl halides:

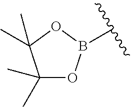 [SMILES: *B1OC(C)(C)C(C)(C)O1]

In many embodiments, the R¹ radicals are aryl or heteroaryl radicals that can themselves be optionally substituted. For example, R¹ can be a C₁-C₃₀ aryl (such as phenyl, napthyl, biphenyl, and the like as described elsewhere herein), or heteroaryl (such as thiophene, pyrrole, thiazole, or the like as described elsewhere herein), optionally substituted by one to four ring substituents independently selected from halides, alkyl, alkynyl, cyano, perfluoroalkyl, alkoxide, perfluoroalkoxide, —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group to form a ring bridging the oxygen atoms.

In some embodiments, R¹ can be an optionally substituted C₁-C₃₀ alkynyl radical, such as those having the structure —C≡C—R², wherein R² can be hydrogen, —Si(R²)₃, wherein each R² is an independently selected alkyl or aryl, or an optionally substituted alkyl, aryl, or heteroaryl.

In some preferred embodiments, the R¹ radicals can be either optionally substituted aryl or heteroaryls having a relatively electron-rich conjugated π electron system that can function as “electron donor” “co-monomer”, or a relatively electron-poor conjugated π electron system that can function as “electron acceptor” “co-monomer”, for the preparation of oligomeric compounds that are useful for making downstream “low bandgap” copolymers capable of efficiently conducting holes or electrons. Non-limiting examples of desirable electron rich R¹ radicals include the various heteroaryls shown below:

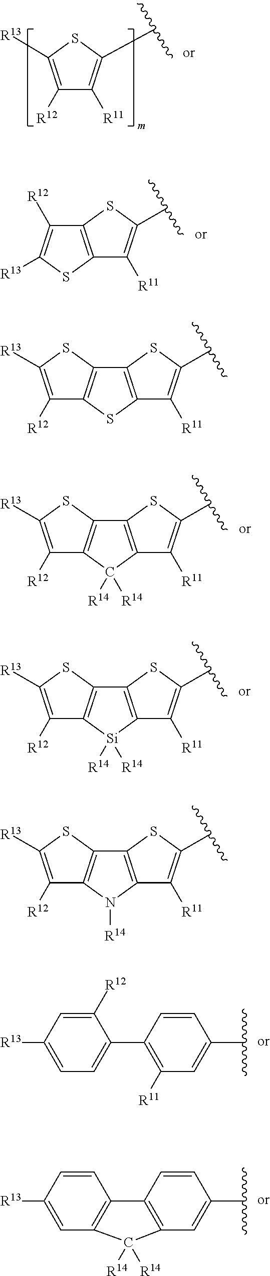

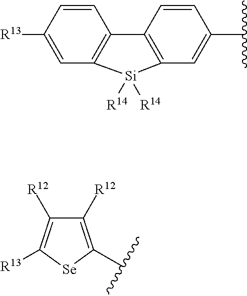

R¹ can also be a relatively electron poor heteroaryl radical, such as for example one of the formulas shown below:

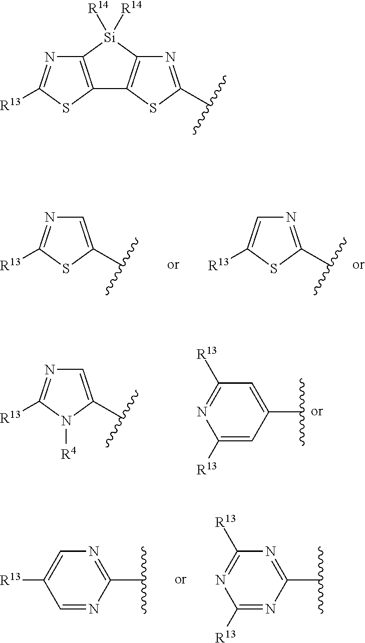

In connection with substituents for the R¹ aryl or heteroaryl groups described above, R¹¹, R¹², R¹⁴ can be any C₁-C₃₀ organic radical, such as but not limited to a C₁-C₁₈ alkyl, perfluoroalkyl, or alkoxy group, and R¹³ can be hydrogen, halide, any C₁-C₃₀ organic radical, such as but not limited to a C₁-C₁₈ alkyl, perfluoroalkyl, or alkoxy group, including Si(R²)₃, Si(OR²)₃, —B(—OR²¹)₂, or Sn(R²)₃.

In many embodiments, the R¹ radicals are “terminal” aryl or heteroaryl radicals, such as the electron poor radicals shown below:

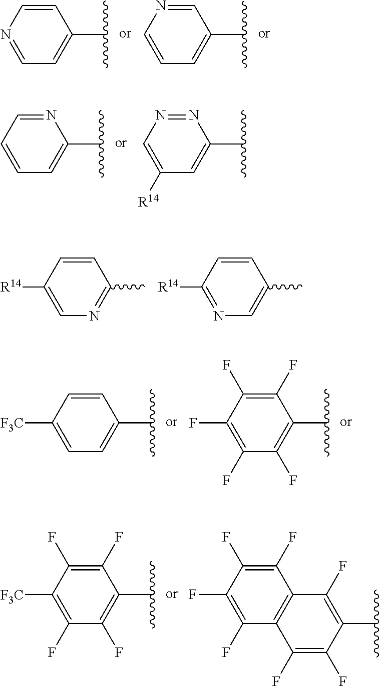

In additional related embodiments, the precursor compounds used for the synthesis of compounds of Formula (I) can have the structure shown below:

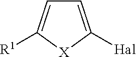

wherein
a. R¹ and Hal can be defined in any of the ways described above; and
b. X is S, Se or NR³ wherein R³ is a C₁-C₁₈ alkyl, perfluoroalkyl, aryl, or heteroaryl. In some embodiments, R³ is CF₃.

In additional related embodiments, the precursor compounds used for the synthesis of compounds of Formula (I) can be the thiazole or imidazole

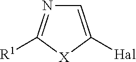

wherein
a. R¹ and Hal can be defined in any of the ways described above; and
b. X is S or NR³ wherein R³ is a C₁-C₁₈ alkyl, perfluoroalkyl, aryl, or heteroaryl.

In some embodiments, R³ is CF₃.

In additional related embodiments, the precursor compounds used for the synthesis of compounds of Formula (I) can be the thiazoles shown below:

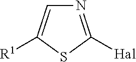

wherein
a. R¹ and Hal can be defined in any of the ways described above.

It will be appreciated that many methods for synthesizing many of the various precursor compounds described above are known to those of ordinary skill in the art, or are commercially available from well known suppliers. Exemplary methods for synthesizing some of the precursor compounds are provided below. It should also be noted that one kind of R¹ substituent (such as —SiR₃ groups) may be initially present before the Halogen Dance/oxidative coupling reaction sequences are employed, but that the initial R¹ group (such as halide or —SiR₃ groups) could then be optionally removed and replaced with a different R¹ group, such as an aryl or heteroaryl, or SnR₃ or organoborate ester group.

The method for synthesizing the bishalo-bisheteroaryl compounds of Formula (I) described and claimed herein typically comprise at least the following steps, which relates to performance of the Base-Catalyzed Halogen Dance portion of the reaction sequence:
a. treating the precursor compound with a strongly basic compound to induce the isomerization of the precursor compound to produce an intermediate compound wherein the Hal atom is bound to a different position on the HAr ring;

The strongly basic compounds used to initiate the “Base-Catalyzed Halogen Dance” reaction can be any compound that is sufficiently strongly basic to deprotonate one of the ring hydrogens of the precursor compound, to form the reactive equivalent of an organic anion on the deprotonated carbon in the ring of the precursor compound. In practice, the strongly basic compounds employed are typically organometallic compounds of Group I or Group II metals, especially organolithium or organomagnesium compounds. In many embodiments, the strongly basic compound employed can be a lithium dialkylamide (such as for example lithium diisopropyl amide).

Typically, the “Base-Catalyzed Halogen Dance” rearrangement reaction corresponding to step b recited above is initiated by addition of a small molar excess (for example about 1.1 equivalents) of the strongly basic compound to a solution of the precursor compound. Without wishing to be bound by theory, it is believed that this practice typically results in the deprotonation of a hydrogen atom of the precursor compound and concurrent formation of an organometallic (usually lithium) salt of the precursor compound as a highly reactive “in-situ” intermediate, which undergoes isomerization to form thermodynamically more stable species. During the reaction, the base present also initiates a sequence of lithium-halogen exchange reactions, which can have the effect of moving/isomerizing the halogen atom of the precursor compound (Hal) to a more thermodynamically stable position on the ring of the precursor compound. This “Base-Catalyzed Halogen Dance” reaction sequence, which produces a highly reactive organometallic intermediate compound wherein the Hal atom is bound to a different position on the HAr ring” can be conceptually illustrated by the diagram below:

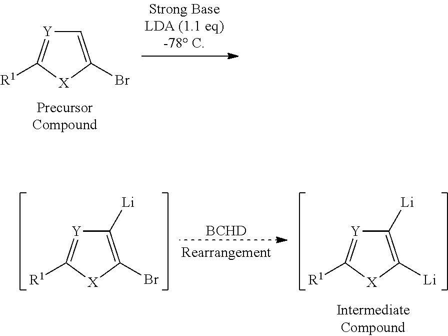

In the methods of the invention, the rearranged and often highly reactive intermediate compound is then subjected to an oxidative coupling step, as recited below.
a. treating the intermediate compound with an oxidizing agent so as to form a carbon-carbon bond between two intermediate compounds and thereby form the bishalo-bisheteroaryl compound.

A wide variety of oxidizing agents can be used to treat the intermediate compound and form the bishalo-bisheteroaryl compound. For example, thionyl chloride and a variety of copper (II) salts can be employed. CuCl₂ is employed as an oxidizing agent in many embodiments of the methods of the invention. A schematic diagram illustrating the oxidation reaction and formation of the bishalo-bisheteroaryl compound is shown below.

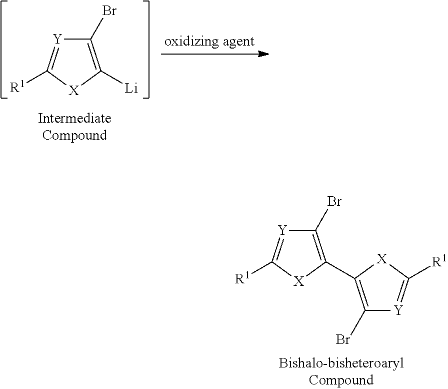

The product bishalo-bisheteroaryl compounds can be readily purified and isolated by many of the methods well known in the art, including extraction, distillation, crystallization, sublimation, or chromatography.

A general synthetic procedure for carrying out some of the synthetic methods described above and claimed below is as follows: A heteroaryl bromide is dissolved in anhydrous THF and the solution cooled in acetone/dry ice bath under nitrogen atmosphere. Lithium diisopropyl amide (LDA) (1.1 eq.) is added dropwise and the progress of the BCHD reaction monitored by GC/MS and/or ¹H NMR. After BCHD reaction completion, CuCl₂ (1.1 eq.) is added in one portion, the mixture stirred at −78° C. for a few hours and then warmed to room temperature. The reaction mixture is diluted with hexanes and water, the organic phase is removed and the aqueous phase is extracted with hexanes several times. The combined organic phases are dried over MgSO₄, the solvents were removed by rotary evaporation, the residue is dissolved in hexanes or other suitable solvent and the solution is filtered through a plug of silica gel. The product can be further purified by crystallization, sublimation, column chromatography, Kugelrohr distillation, or many other techniques well known to those of ordinary skill in the art.

Examples of sub-generic classes of bishalo-bisheteroaryl compounds that can be synthesized via the methods of the invention have Formula (Ia) shown below:

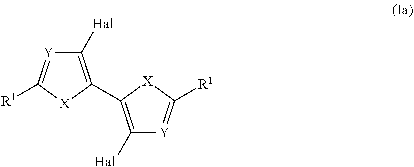

wherein R¹, X, Y, and Hal can be defined in any of the ways already detailed above, or as follows:
a. R¹ is a halide, or a C₁-C₃₀ organic radical selected from optionally substituted alkyl, alkynyl, aryl, and heteroaryl, or —Sn(R²)₃, —Si(R²)₃, —Si(OR²)₃, or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b. X is O, S, Se, or NR³ wherein R³ is a C₁-C₁₈ alkyl, perfluoroalkyl, aryl, or heteroaryl; and
c. Y is CH, CR⁴, or N, wherein R⁴ is a C₁-C₁₈ alkyl, aryl, or heteroaryl;

In Formulas I, Ia, and their various subgenera described herein, Hal can be a halogen, including F, Cl, Br, or I. In many embodiments Hal is Br or I, or in many cases Br.

Examples of other sub-generic classes of bishalo-bisheteroaryl compounds that can be synthesized via the methods of the invention are shown below:

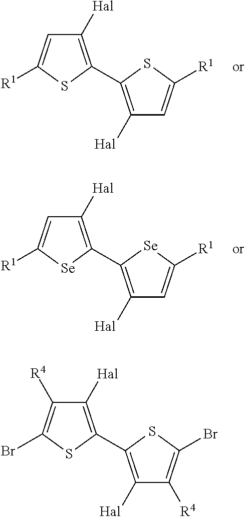

wherein R¹, X, Y, and Hal can be defined in any of the ways already detailed above, especially wherein Hal is Br, or as follows:
a. R¹ is a halide, or a C₁-C₃₀ organic radical selected from optionally substituted alkyl, alkynyl, aryl, heteroaryl, or —Sn(R²)₃, —Si(R²)₃, —Si(OR²)₃, or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b. R⁴ is a C₁-C₁₈ alkyl, aryl, or heteroaryl.

Additional examples of sub-generic classes of bishalo-bisheteroaryl compounds that can be synthesized via the methods of the invention are shown below:

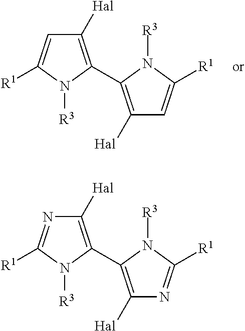

wherein R¹, X, Y, and Hal can be defined in any of the ways already detailed above, or as follows:
a. R¹ is a halide, or a C₁-C₃₀ organic radical selected from optionally substituted alkyl, alkynyl, aryl, heteroaryl, or —Sn(R²)₃, —Si(R²)₃, Si(OR²)₃, or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b. R³ is a C₁-C₁₈ alkyl, perfluoroalkyl, aryl, or heteroaryl.

Yet further examples of sub-generic classes of bishalo-bisheteroaryl compounds that can be synthesized via the methods of the invention are shown below:

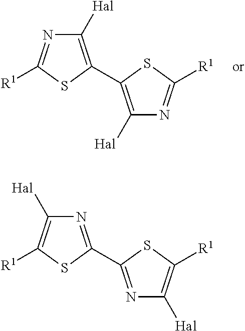

wherein R¹, X, Y, and Hal can be defined in any of the ways already detailed above, or wherein R¹ or a C₁-C₃₀ organic radical selected from optionally substituted alkyl, alkynyl, aryl, heteroaryl, or —Sn(R²)₃, —Si(R²)₃, Si(OR²)₃, or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms.

It should also be noted that for any of the sub-generic classes of bishalo-bisheteroaryl compounds described above, in some embodiments R¹ can have the structures shown below:

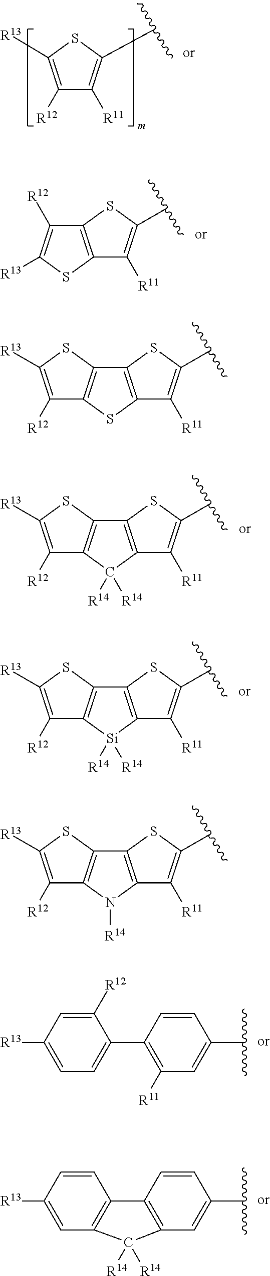

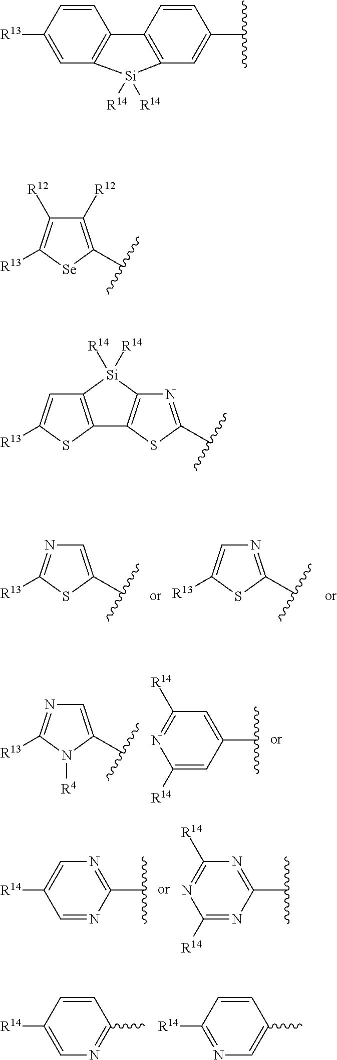

wherein m is 1, 2, 3, or 4, and R¹¹, R¹², R¹⁴ are hydrogen or a C₁-C₁₈ alkyl, perfluoroalkyl, alkoxy, or perfluoroalkoxy group, and R¹³ is hydrogen, —B(—OR²¹)₂, Si(R²)₃, or Sn(R²)₃, wherein each R² is an independently selected alkyl or aryl, and each R²¹ is an independently selected alkyl or aryl, or the R²¹ groups together form an optionally substituted alkylene group to form a ring bridging the oxygen atoms.

Suitable starting materials for preparing compounds of Formula (I) having two thiazole rings and having a variety of aryl or heteroaryl substituents at R¹ can often be prepared by the generic synthetic procedure illustrated in the diagram below:

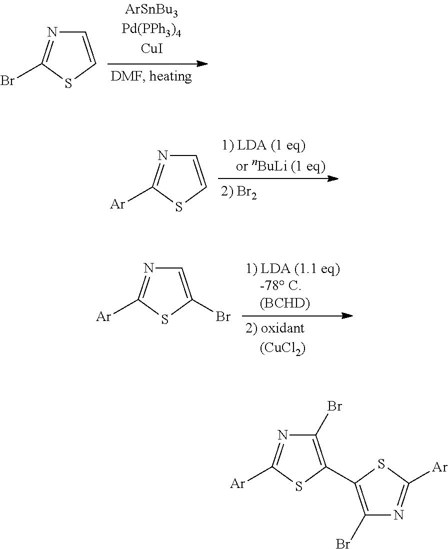

Specific examples of bishalo-bisheteroaryl compounds that have been synthesized by the methods of the invention are shown in Table 1 shown below, and further examples provided in the Examples Section below.

TABLE 1
Examples of compounds synthesized via the sequence of BCHD rearrangement—CuCl₂ oxidative coupling.

| | | | Isolated |
|---|---|---|---|
| Entry | Substrate | Product | Yield, % |
| 1 | 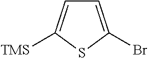 | 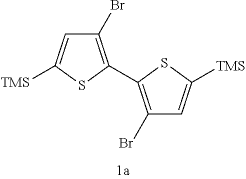 [SMILES: C[Si](C)(C)c1cc(Br)c(-c2sc([Si](C)(C)C)cc2Br)s1] | 60-84 |
| 2 | 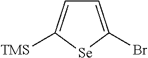 | 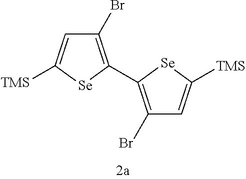 [SMILES: C[Si](C)(C)c1cc(Br)c(-c2[se]c([Si](C)(C)C)cc2Br)[se]1] | 50-66 |
| 3 | 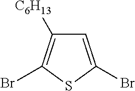 | 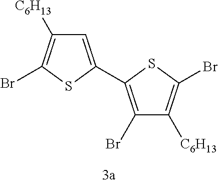 | 81-87 |
| 4 | 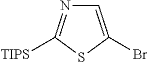 | 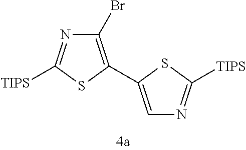 | 60-82 |
| 5 | 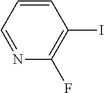 | 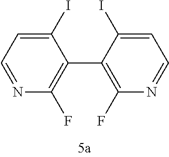 [SMILES: Fc1nccc(I)c1-c1c(I)ccnc1F] | 60 |
| 6 | 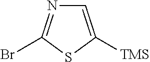 | 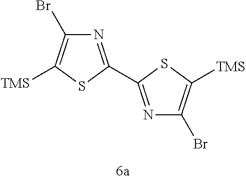 [SMILES: C[Si](C)(C)c1sc(-c2nc(Br)c([Si](C)(C)C)s2)nc1Br] | 35-68 |
| 7 | 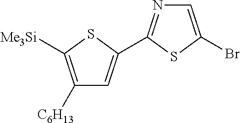 | 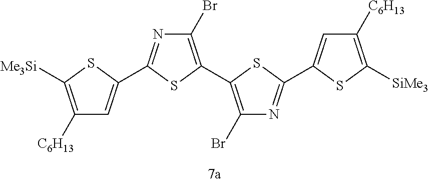 | 67% (after l^{st} column) |

### Methods for Synthesizing Fused Tricyclic Compounds

The ready availability of a wide variety of bishalo-bisheteroaryl compounds of Formula (I) via the synthetic methods described above provides a wide variety of starting materials for the synthesis of a wide variety of fused tricyclic compounds of Formula (II), as shown below:

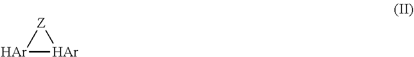

wherein
a. HAr can be any of the optionally substituted heteroaryl ring radicals disclosed elsewhere herein, and
b. Z is a bridging group, such as S, Se, NR⁵, C(O), C(O)C(O), Si(R⁵)₂, SO, SO₂, PR⁵, P(O)R⁵, BR⁵, or C(R⁵)₂ wherein R⁵ is an organic radical.

Many fused tricyclic compounds of Formula (II) can be prepared by additionally
a. optionally treating the bishalo-bisheteroaryl compound with an organometallic compound to exchange a metal for the Hal substituents, and form a bismetallo-bisheteroaryl compound, and
b. reacting the bismetallo-bisheteroaryl compound with a suitable electrophile, or reacting the bishalo-bisheteroaryl compound or bismetallo-bisheteroaryl compound with a nucleophile, to introduce the Z group, or a precursor thereof suitable for forming the fused tricyclic compound.

Restating the method steps above, in some embodiments, the invention relates to multi-step methods of making fused tricyclic compounds of Formula (II), comprising the structure

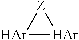

wherein
a. HAr is an optionally substituted five or six membered heteroaryl ring comprising at least one ring carbon atom and at least one ring heteroatom,
b. Z is S, Se, NR⁵, C(O), C(O)C(O), Si(R⁵)₂, SO, SO₂, PR⁵, P(O)R⁵, BR⁵, or C(R⁵)₂ wherein R⁵ is a C₁-C₅₀ organic radical selected from optionally substituted alkyl, perfluoroalkyl, aryl, and heteroaryl,
   wherein the method comprises the steps of
i) providing an optionally substituted precursor compound comprising a halo-heteroaryl ring having an Hal substituent at a first position on the HAr ring, and Hal is a halogen, and
ii) treating the precursor compound with a strongly basic compound to induce the isomerization of the precursor compound to produce an intermediate compound wherein the Hal atom is bound to a different position on the HAr ring; and
iii) treating the intermediate compound with an oxidizing agent so as to form a carbon-carbon bond between two intermediate compounds and thereby form the bishalo-bisheteroaryl compound having the structure

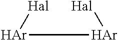

and
iv) optionally treating the bishalo-bisheteroaryl compound with an organometallic compound to exchange a metal for the Hal substituents, and form a bismetallo-bisheteroaryl compound, and
   (1) reacting the bismetallo-bisheteroaryl compound with a suitable electrophile, or
   (2) reacting the bishalo-bisheteroaryl compound or bismetallo-bisheteroaryl compound with a nucleophile, to introduce the Z group, or a precursor thereof suitable for forming the fused tricyclic compound.

In some embodiments of such methods of making the fused tricyclic compounds of Formula (I), the halogenated positions of the bishalo-bisheteroaryl compounds of Formula (I) can condensed with nucleophilic reagents that comprise the Z group. Consider for example the following exemplary condensation reaction of a bishalo-bisheteroaryl compound with a nucleophilic amine compound in the presence of a palladium catalyst, whose details are presented below in Example 10. or a similar novel selenium derivative:

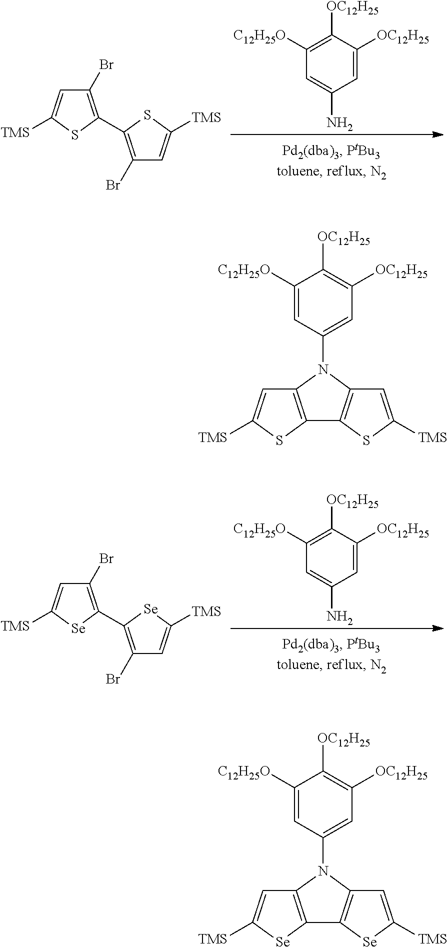

In other embodiments of the methods of making the fused tricyclic compounds, the bishalo-bisheteroaryl compound is first reacted with an organometallic compound to exchange a metal for the Hal substituents, and thereby form a nucleophilic bismetallo-bisheteroaryl compound, which is then condensed with an electrophilic source of the Z radical, to form a subclass of fused tricyclic compounds of Formula (IIa), as shown below:

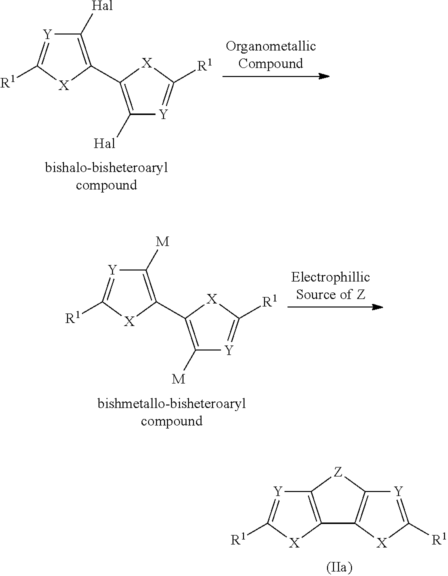

As indicated in the diagram above, the organometallic compound used to react with and activate the bishalo-bisheteroaryl compound and form a bismetallo-bisheteroaryl compound, which is then reacted with a suitable source of the Z radical. Suitable organometallic compounds for activating the bishalo-bisheteroaryl compound include highly basic and/or nucleophilic main group organometallic compounds such as organolithium compounds (such as n-butyl lithium), or organomagnesium compounds. Other suitable organometallic compounds for activating the bishalo-bisheteroaryl compound include various transition metal catalyst compounds, especially late transition metals from Groups VIII, IB, or IIB.

In many embodiments of the methods, the electrophilic source of the Z radical can be a compound V—R⁶—V′, where R⁶ is selected from S, Se, NR⁵, C(O), C(O)C(O), Si(R⁵)₂, SO, SO₂, PR⁵, P(O)R⁵, BR⁵, or C(R⁵)₂, and V and V′ are leaving groups, or V and V′ together form a leaving group suitable for a condensation reaction with the bismetallo-bisheteroaryl compound, to form the fused tricyclic compound. In many embodiments, R⁵ is an optionally substituted organic radical selected from alkyl, perfluoroalkyl, alkoxide, aryl, heteroaryl, or the like. R⁵ has between one and 50 carbon atoms, or between 2 and 30 carbon atoms. In many embodiments, V and/or V′ are halides such as Cl, Br or I, or other similar anionic leaving groups.

Specific examples of suitable V—R⁶—V′ reagents for introducing the Z radicals include but are not limited to dimethylcarbamoyl chloride (for introducing a CO group), diethyl oxalate (for introducing α-dicarbonyl groups), Cl₂SiR₂ (for introducing SiR₂ groups), SCl₂ or (PhSO₂)₂S (for introducing S bridges, which can be oxidized to SO or SO₂ groups), RB(OMe)₂ (for introducing BR bridges); Cl₂PR (for introducing PR bridges, which can be oxidized to phosphine oxides); and (PhSO₂)₂Se (for introducing Se bridges).

In other embodiments, V and/or V′ can be organic leaving groups, such as perfluoroalkoxides, or amines such as the N,N-dimethylethylenediamine radical of N,N-dimethyl-piperazine-2,3-dione, which is an effective source of alpha-dicarbonyl “Z” groups, as illustrated by the drawing and Example 16 below.

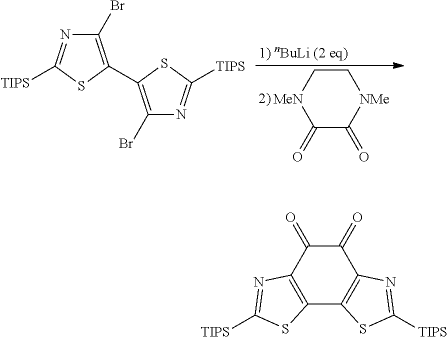

Overall, the various inventions described herein relate to general three step method for synthesizing a very wide variety of fused tricyclic compounds, as shown in the reaction scheme diagram below:

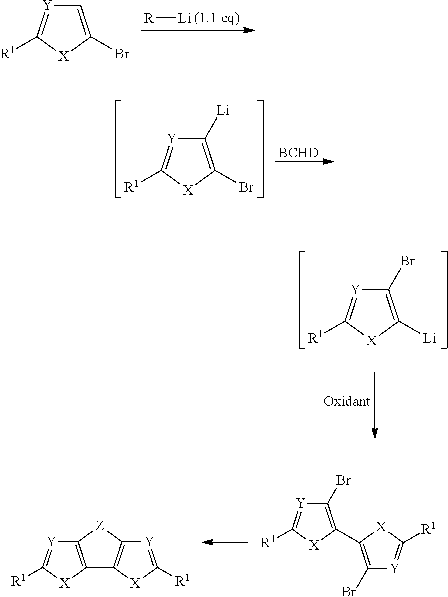

wherein R¹, X, Y, and Z can be defined in any of the ways disclosed hereinabove.

### The Fused Tricyclic Compounds

As disclosed and described above, the various embodiments of the methods of the inventions provide unexpectedly short, efficient and inexpensive methods for making a wide variety of fused tricyclic compounds, many of which can be used as semiconducting materials for making electronic devices, or they may be used as synthetic intermediates and further elaborated or polymerized to produce other semiconducting materials useful for making electronic devices.

The fused tricyclic compounds that can be made by the methods described herein include some have the general structure of Formula (II) shown below:

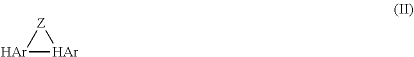

wherein
a. HAr can be defined in any manner described above, and
b. Z is an organic or inorganic group bridging the two HAr radicals to form the tricyclic compound. For example, Z can be S, Se, NR⁵, C(O), C(O)C(O), Si(R⁵)₂, SO, SO₂, PR⁵, P(O)R⁵, BR⁵, or C(R⁵)₂ wherein R⁵ is an optionally substituted organic radical selected from alkyl, perfluoroalkyl, alkoxide, aryl, heteroaryl, or the like. It should also be noted that when Z is C(O) or C(O)C(O) (i.e. one or more carbonyl groups, the corresponding ketals can also be readily synthesized, as disclosed below, and such ketals can be very valuable synthetic intermediates that facilitate additional functionalization of the HAr groups, as will also described below.

In many embodiments of the fused tricyclic compounds, HAr is an optionally substituted five membered heterocycle. Examples of the such fused tricyclic compounds can have the generic structure shown in Formula (IIa) shown below

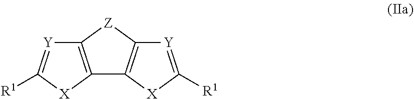

wherein R¹, X, Y, and Z can be defined in any of the ways disclosed herein.

In some such embodiments of the compounds of Formula (IIa), R¹ can be hydrogen, a halide, or a C₁-C₃₀ organic radical. Such R¹ organic radicals can be selected from optionally substituted alkyl, alkynyl, aryl, and heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms. Such R¹ organic radicals can be selected from an organic acyl compound having the formula

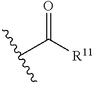

wherein R¹¹ is an aryl or heteroaryl optionally substituted with 1-10 independently selected halide, cyano, alkyl, perfluoroalkyl, acyl, alkoxy, or perfluoroalkoxy groups.

In the compounds of Formula (IIa),
a. X can be O, S, Se, or NR³ wherein R³ is a C₁-C₁₈ alkyl, perfluoroalkyl, aryl, or heteroaryl; and
b. Y can be CH, CR⁴, or N, wherein R⁴ is a C₁-C₁₈ alkyl, aryl, or heteroaryl; and
c. Z can be S, Se, NR⁵, C(O), C(O)C(O), Si(R⁵)₂, SO, SO₂, PR⁵, P(O)R⁵, BR⁵, or C(R⁵)₂, wherein R⁵ is a C₁-C₅₀ organic radical selected from optionally substituted alkyl, perfluoroalkyl, aryl, and heteroaryl.

In some preferred embodiments the compounds of Formula (IIa), Z is C(O), C(O)C(O), to give mono or bis keto derivatives of Formula (IIb) or Formula (IIb), or ketal protected derivatives thereof, having Formulas (IId), (IIe), or (IIf) shown below, where n is 2 or 3.

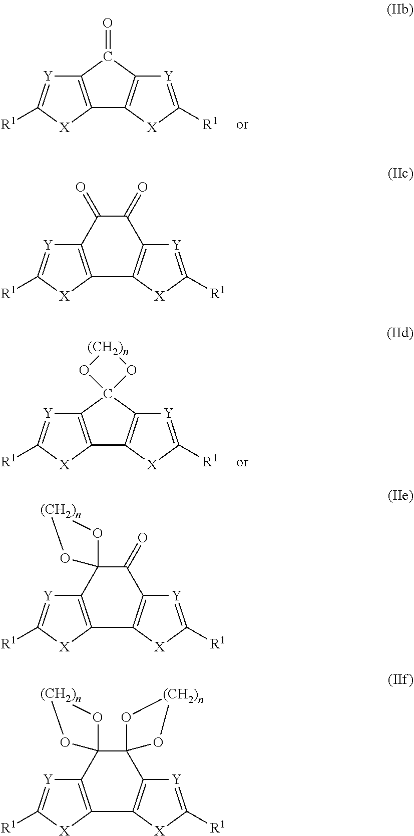

wherein X, Y, and R¹ can be any of the groups identified elsewhere herein.

The ketal protected derivatives having Formulas (IId), (IIe), or (IIf) are especially useful as synthetic intermediates that allow easy further functionalizations at R¹, followed by deprotection to liberate the functionalized parent carbonyl compounds. Specific examples of such ketal protected compounds include the bis-thiophene and bisthiazole ketal compounds whose structures are shown below:

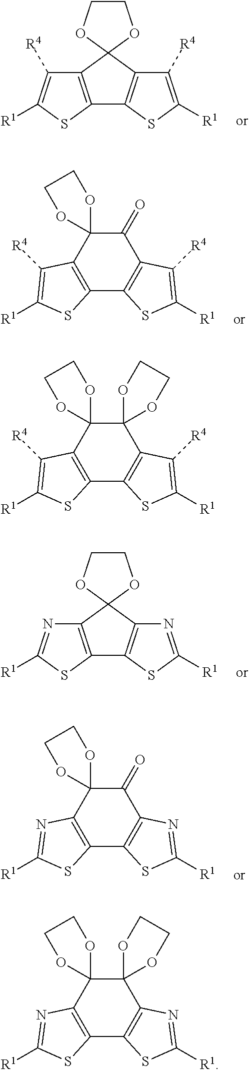

Some subgenera of the compounds of Formulas (IIa), (IIb), and (IIc) include the bis-thiophenes having the structure

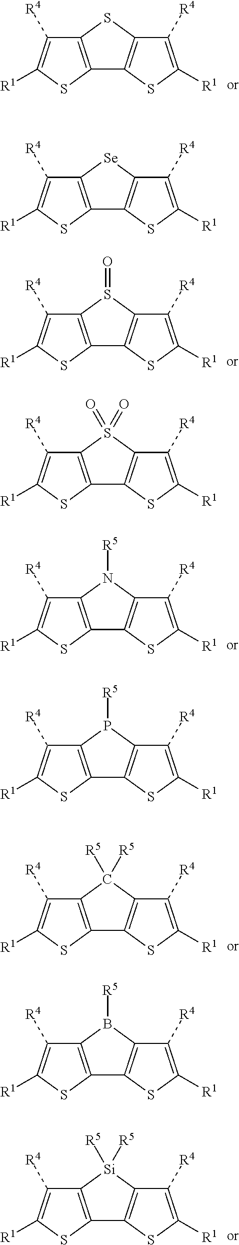

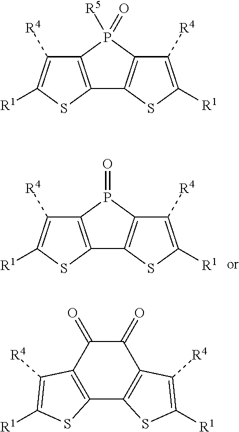

wherein R¹ can be hydrogen or a halide, or a C₁-C₃₀ organic radical selected from optionally substituted alkyl, alkynyl, aryl, and heteroaryl, or —Sn(R²)₃, —Si(R²)₃, Si(OR²)₃ or —B(—OR²¹)₂ wherein each R² can be an independently selected alkyl or aryl, and each R²¹ can be an independently selected alkyl or aryl, or the R²¹ groups together form an optionally substituted alkylene group bridging the oxygen atoms, R⁴ can be hydrogen or optionally a C₁-C₁₈ alkyl group, and R⁵ can be a C₁-C₅₀ organic radical selected from alkyl, aryl, heteroaryl.

Related subgenera of the compounds of Formula (IIa) include the bis-selenophenes having the structure

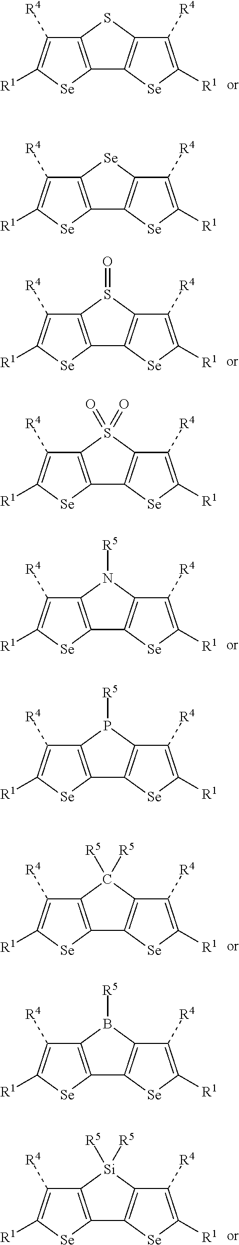

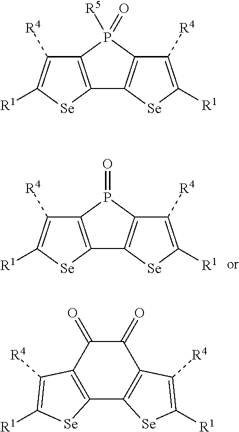

wherein R¹ can be hydrogen or a halide, or a C₁-C₃₀ organic radical selected from optionally substituted alkyl, alkynyl, aryl, and heteroaryl, or —Sn(R²)₃, —Si(R²)₃, Si(OR²)₃ or —B(—OR²¹)₂ wherein each R² can be an independently selected alkyl or aryl, and each R²¹ can be an independently selected alkyl or aryl, or the R²¹ groups together form an optionally substituted alkylene group bridging the oxygen atoms, R⁴ can be hydrogen or optionally a C₁-C₁₈ alkyl group, and R⁵ can be a C₁-C₅₀ organic radical selected from alkyl, aryl, heteroaryl.

Other related embodiments of the compounds of Formula (IIa) include the bispyrroles shown below:

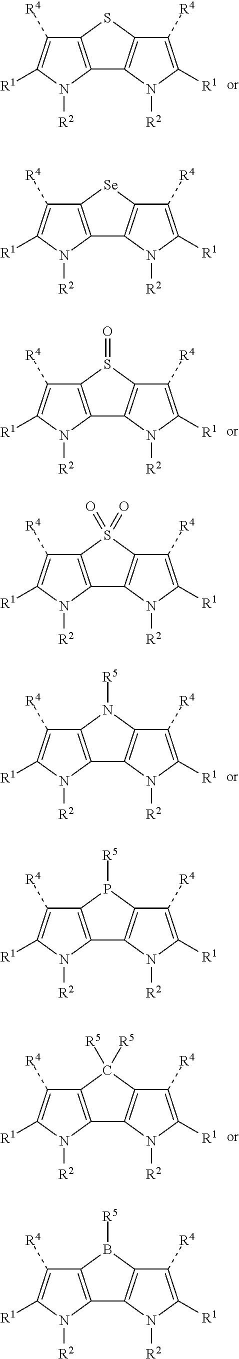

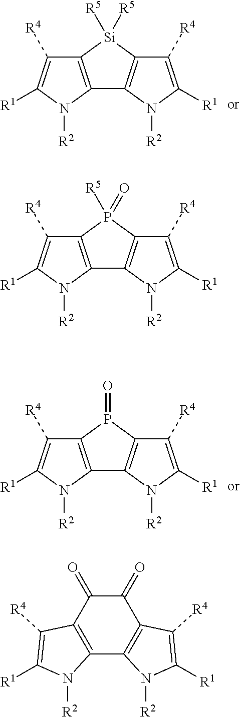

wherein R¹ is hydrogen or a halide, or a C₁-C₃₀ organic radical selected from alkyl, alkynyl, aryl, or heteroaryl, or —Sn(R²)₃, —Si(R²)₃, Si(OR²)₃ or —B(—OR²¹)₂ wherein each R² is an independently selected alkyl, perfluoroalkyl, or aryl and each R²¹ is an independently selected alkyl or aryl, or the R²¹ groups together form an optionally substituted alkylene group to form a ring bridging the oxygen atoms, R⁴ is hydrogen, cyano, or optionally a C₁-C₁₈ alkyl group, and R⁵ is a C₁-C₅₀ organic radical selected from alkyl, aryl, heteroaryl. In some embodiments, R² is a CF₃ group.

Other related embodiments of the compounds of Formula (IIa) include the bisthiazoles shown below:

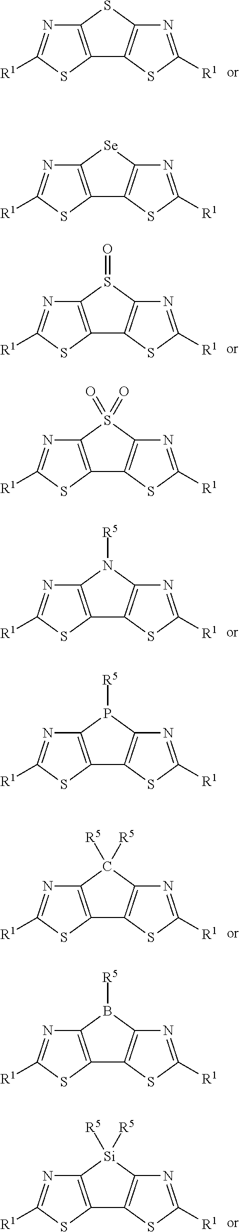

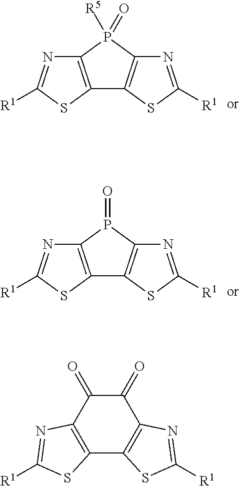

wherein R¹ is hydrogen or a halide, or a C₁-C₃₀ organic radical selected from optionally substituted alkyl, alkynyl, aryl, and heteroaryl, or —Sn(R²)₃, —Si(R²)₃, Si(OR²)₃ or —B(—OR²¹)₂ wherein each R² is an independently selected alkyl or aryl and each R²¹ is an independently selected alkyl or aryl, or the R²¹ groups together form an optionally substituted alkylene group bridging the oxygen atoms, and R⁵ is a C₁-C₅₀ organic radical selected from alkyl, aryl, heteroaryl.

Of particular interest are bisthiazole-biscarbonyl compounds having the structure:

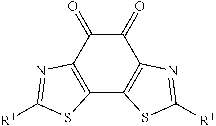

wherein R¹ can be hydrogen, a halide, an optionally substituted C₁-C₃₀ aryl or heteroaryl, alkynyl, Si(R²)₃, Si(OR²)₃, Sn(R²)₃, or B(OR²)₂ wherein each R² is an independently selected C₁-C₁₈ alkyl or aryl, or the R² groups together form a cyclic alkylene.

Such bisthiazole-biscarbonyl compounds have fused tricyclic cores that are highly electron deficient, and are useful for making polymers and/or compositions that can conduct electrons, and hence are very useful for making electronic devices. In addition they can be useful as optical absorbing materials, nonlinear optical materials, sensing materials and optical limiting materials.

Yet other related embodiments of the compounds of Formula (IIa) include the bisimidazoles shown below:

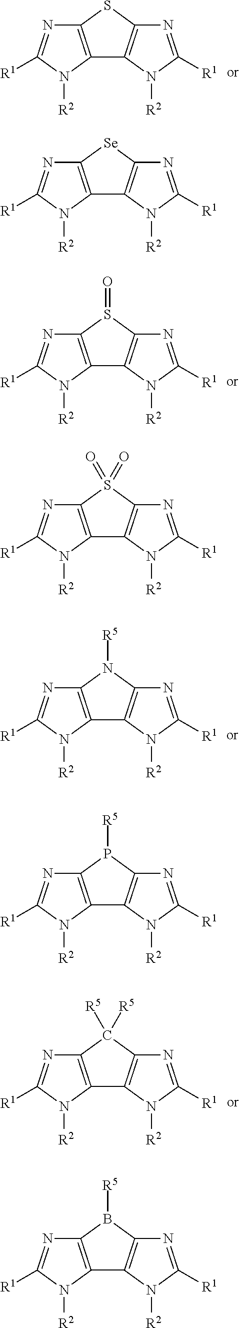

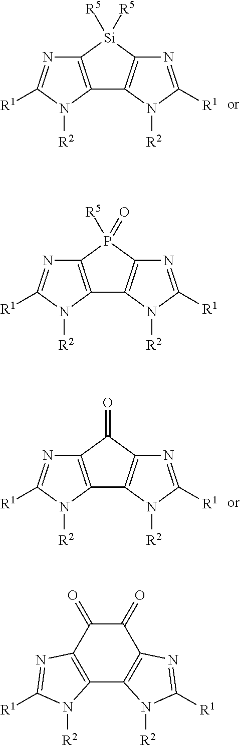

wherein R¹ is hydrogen or a halide, or a C₁-C₃₀ organic radical selected from optionally substituted alkyl, alkynyl, aryl, heteroaryl, or —Sn(R²)₃, —Si(R²)₃, or —B(—OR²¹)₂ wherein each R² is an independently selected alkyl or aryl and each R²¹ is an independently selected alkyl or aryl, or the R²¹ groups together form an optionally substituted alkylene group to form a ring bridging the oxygen atoms, and R⁵ is a C₁-C₅₀ organic radical selected from alkyl, perfluoroalkyl, aryl, or heteroaryl.

In many embodiments of the compounds of Formula (IIa) and its several subgenera shown above, R¹ can be an optionally substituted aryl, or heteroaryl. For example, R¹ can be a relatively electron rich radical having one of the formulas shown below:

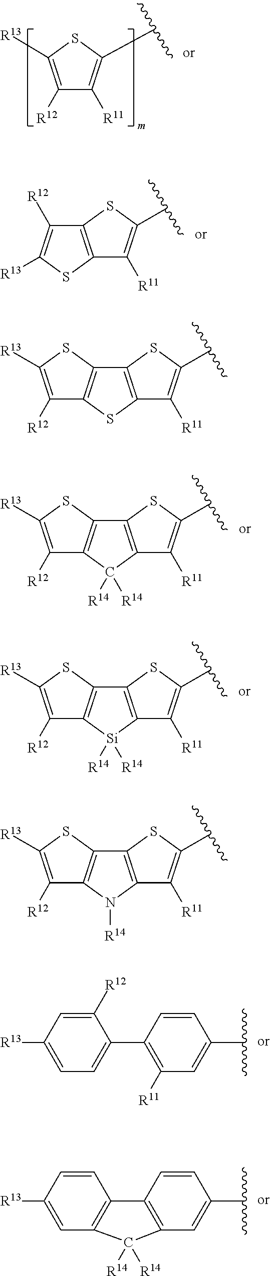

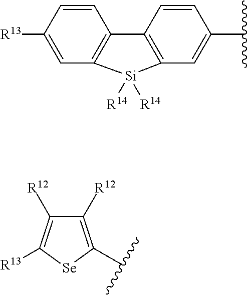

wherein m is 1, 2, 3, or 4, and R⁴, R¹¹, R¹², R¹⁴ are a C₁-C₁₈ alkyl, perfluoroalkyl, or alkoxy group, and R¹³ is hydrogen, halide, Si(R²)₃, Si(OR²)₃ or Sn(R²)₃.

In other embodiments of the fused tricyclic compounds of Formula (IIa) or its subgenera, R¹ can be a relatively electron poor heteroaryl radical, such as for example one of the formulas shown below:

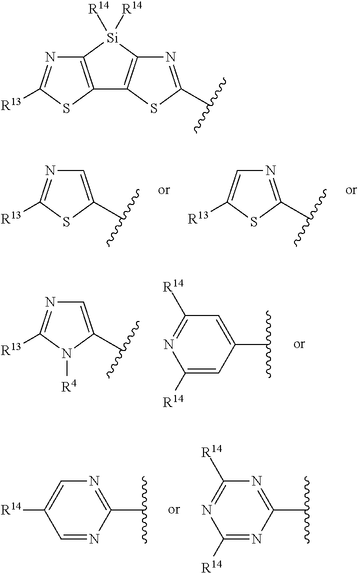

wherein m is 1, 2, 3, or 4, and R⁴, and R¹⁴ are a C₁-C₁₈ alkyl, perfluoroalkyl, or alkoxy group, and R¹³ is hydrogen, halide, Si(R²)₃, or Sn(R²)₃.

Moreover, in some embodiments of the compounds of Formula (IIa), R¹ can be a relatively electron poor terminal aryl or heteroaryl, such as those having the structures:

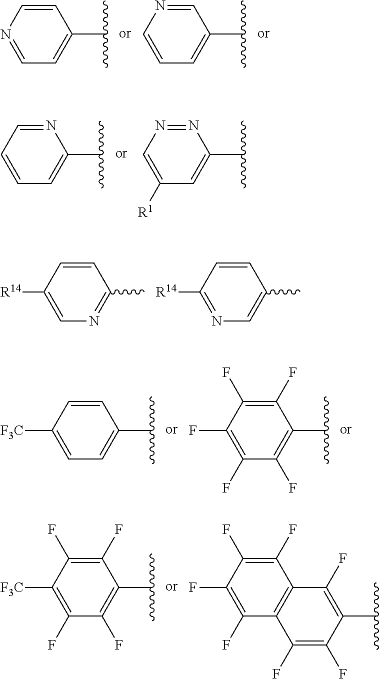

Examples of specific compounds of Formula (IIa) that have been experimentally synthesized in the lab include the compounds illustrated in Table 2.

TABLE 2
Summary of the tricyclic cores obtained from the aryl dibromides synthesized by the sequence of BCHD
reaction and CuCl₂ oxidative coupling.

| | | | Isolated |
|---|---|---|---|
| Entry | Bis heteroaryl Halide | Product | Yield, % |
| 1 | 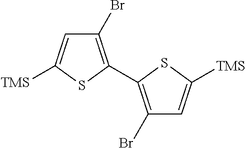 [SMILES: C[Si](C)(C)c1cc(Br)c(-c2sc([Si](C)(C)C)cc2Br)s1] | 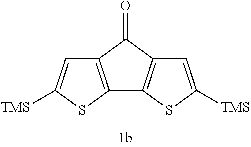 [SMILES: C[Si](C)(C)c1cc2c(s1)-c1sc([Si](C)(C)C)cc1C2=O] | 56-85 |
| 2 | 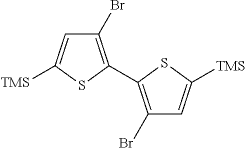 [SMILES: C[Si](C)(C)c1cc(Br)c(-c2sc([Si](C)(C)C)cc2Br)s1] | 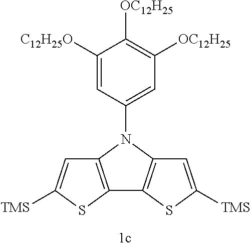 | 53 |
| 1 | 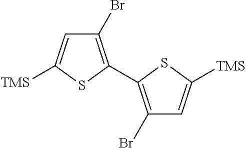 [SMILES: C[Si](C)(C)c1cc(Br)c(-c2sc([Si](C)(C)C)cc2Br)s1] | 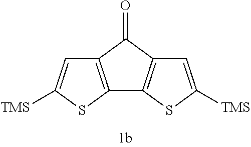 [SMILES: C[Si](C)(C)c1cc2c(s1)-c1sc([Si](C)(C)C)cc1C2=O] | 56-85 |
| 2 | 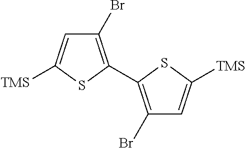 [SMILES: C[Si](C)(C)c1cc(Br)c(-c2sc([Si](C)(C)C)cc2Br)s1] | 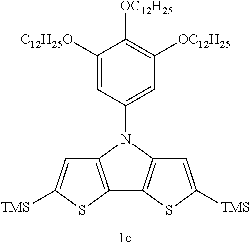 | 53 |
| 3 | 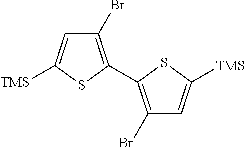 [SMILES: C[Si](C)(C)c1cc(Br)c(-c2sc([Si](C)(C)C)cc2Br)s1] | 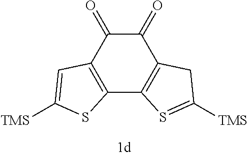 | 27-80 |
| 4 | 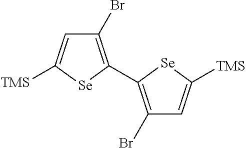 [SMILES: C[Si](C)(C)c1cc(Br)c(-c2[se]c([Si](C)(C)C)cc2Br)[se]1] | 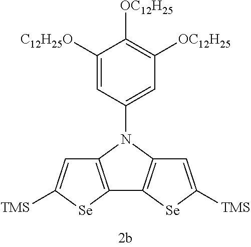 | 51-81 |
| 5 | 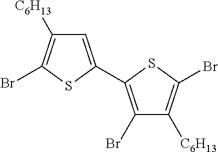 | 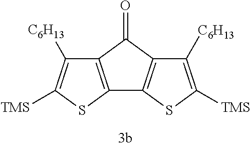 | 52-54 |
| 6 | 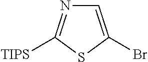 | 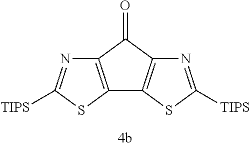 | 39 |
| 7 | 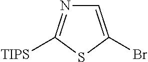 | 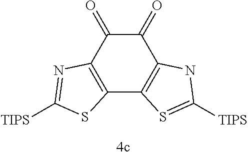 | 38 |
| 1 | 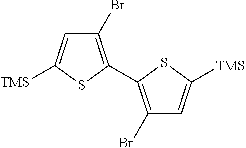 [SMILES: C[Si](C)(C)c1cc(Br)c(-c2sc([Si](C)(C)C)cc2Br)s1] | 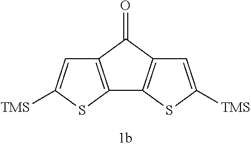 [SMILES: C[Si](C)(C)c1cc2c(s1)-c1sc([Si](C)(C)C)cc1C2=O] | 56-85 |
| 2 | 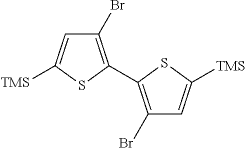 [SMILES: C[Si](C)(C)c1cc(Br)c(-c2sc([Si](C)(C)C)cc2Br)s1] | 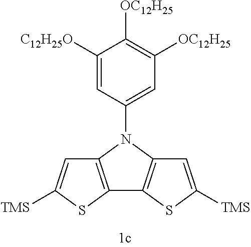 | 53 |
| 8 | 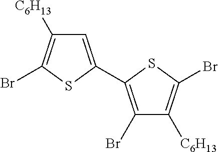 | 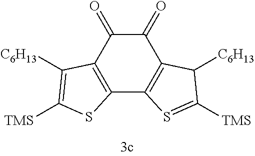 | 34 |

### Compounds of Formula (IIa) as Synthetic Intermediates

The various subgenera of compounds of Formula (II) or (IIa), available by the methods of the invention, can also be readily further functionalized and/or elaborated to produce a wide variety of known and new downstream compounds, oligomers, polymers, or copolymers that are useful for many purposes, including for the preparation of compounds and compositions for making electronic devices, such as transistors, solar cells, light emitting diodes, and the like.

For example, it has been discovered that the compounds of Formula (IIa) wherein R¹ is a triorganosilane can be readily converted to the corresponding iodides or bromides, as shown in the diagram below and in Table 3.

TABLE 3
Summary of Synthesized Fused Tricyclic Dihalides

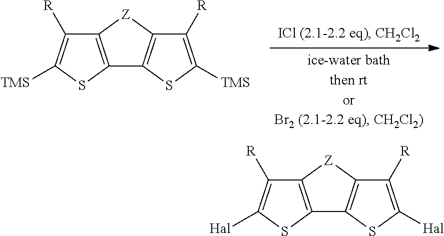

| | | E_{1/2}^{0/-1}, V | E_{1/2}^{-1/-2}, V |
|---|---|---|---|
| Entry | Aryl Dihalide | (solvent) | (solvent) |
| 1 | 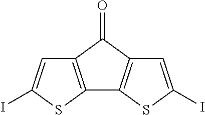 [SMILES: O=C1c2cc(I)sc2-c2sc(I)cc21] | −1.49 (CH₂Cl₂) | n/a |
| 2 | 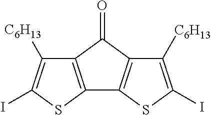 | −1.61 (CH₂Cl₂) | n/a |
| 3 | 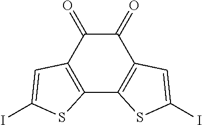 [SMILES: O=C1C(=O)c2cc(I)sc2-c2sc(I)cc21] | −0.94 (THF) | −1.60 (THF) |
| 4 | 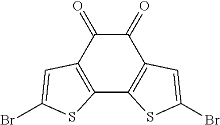 [SMILES: O=C1C(=O)c2cc(Br)sc2-c2sc(Br)cc21] | −0.90 (THF) | −1.64 (THF) |
| 5 | 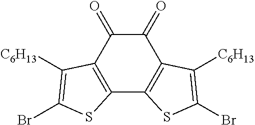 | −1.01 (CH₂Cl₂) | −1.62 (CH₂Cl₂) |
| 6 | 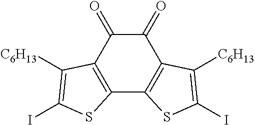 | −1.02 (CH₂Cl₂) | −1.62 (CH₂Cl₂) |
| 7 | 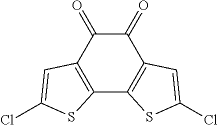 [SMILES: O=C1C(=O)c2cc(Cl)sc2-c2sc(Cl)cc21] | −0.88 (THF) | −1.68 (THF) |
| 8 | 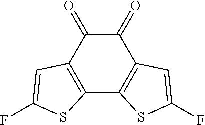 [SMILES: O=C1C(=O)c2cc(F)sc2-c2sc(F)cc21] | −0.91 (THF) | −1.73 (THF) |
| 9 | 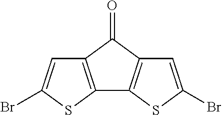 [SMILES: O=C1c2cc(Br)sc2-c2sc(Br)cc21] | −1.48 (CH₂Cl₂) | |

R = H, C₆H₁₃; Z = C(O), C(O)—C(O); Hal = Br, I
CV experiment: 0.1 M ⁿBu₄NPF₆ in THF or CH₂Cl₂ vs Cp₂Fe at 0 V

Such fused tricyclic dihalides can be coupled at the R¹ halides with a wide variety of other aryl or heteroaryl compounds, via the well known Stille, Sonogashira or Suzuki coupling procedures (see Hassan et al. Chem. Rev., 2002, 102, 1359-1469, and Sonogashira et al., Tetrahedron Lett., 1975, 50, 4467-4470, both hereby incorporated herein by reference), to produce a wide variety of oligomers, or polymerizable oligomeric materials that can be used to prepare copolymers comprising those repeat units.

Alternatively, fused tricyclic compounds comprising Si(OR)₃ or SnR₃ radicals suitable for Hiyama or Stille couplings or polymerizations with other corresponding aryl or heteroaryl radicals can be prepare as indicated in the reaction diagrams shown below:

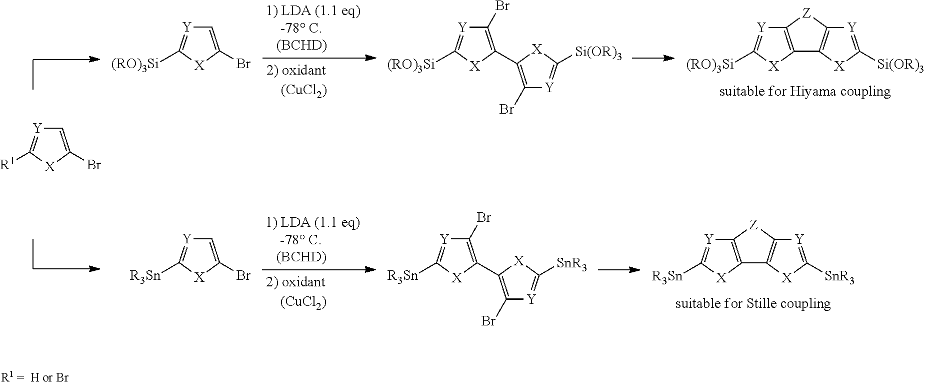

### Polymers Comprising the Fused Tricyclic Compounds as Repeat Units

Some aspects of the present inventions relate to new polymers comprising one or more of the fused tricyclic compounds disclosed herein as repeat units for copolymers. For example, some embodiments of the inventions herein relate to a polymer or copolymer comprising a repeat unit having the structure

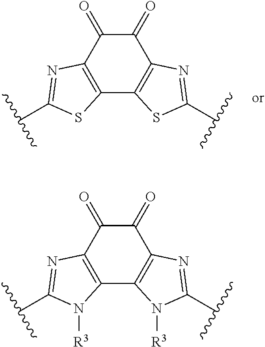

wherein R³ is a C₁-C₁₈ alkyl, perfluoroalkyl, aryl, or heteroaryl. In some embodiments, R³ is CF₃.

In other embodiments, the invention relates to polymers or copolymer comprising a repeat unit having the structure

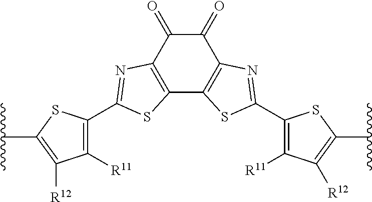

wherein R¹¹ and R¹² are hydrogen or a C₁-C₁₈ alkyl.

Many such polymers or copolymers can be unexpectedly superior organic semiconductors capable of transporting holes and/or electrons, and can be solution processed, so as to be useful in the synthesis of electronic devices, such as transistors, solar cells, and/or organic light emitting diodes.

### EXAMPLES

The various inventions described above are further illustrated by the following specific examples, which are not intended to be construed in any way as imposing limitations upon the scope of the invention disclosures or claims attached herewith. On the contrary, it is to be clearly understood that resort may be had to various other embodiments, modifications, and equivalents thereof which, after reading the description herein, may suggest themselves to one of ordinary skill in the art without departing from the spirit of the present invention or the scope of the appended claims.

General

All experiments with air- and moisture-sensitive intermediates and compounds were carried out under an inert atmosphere using standard Schlenk techniques. NMR spectra were recorded on 400 MHz Bruker AMX 400 and referenced to residual proton solvent or internal tetramethylsilane standard. UV-vis absorption spectra were recorded on a Varian Cary 5E UV-vis-NIR spectrophotometer. Cyclic voltammograms were obtained on a computer controlled BAS 100B electrochemical analyzer, and measurements were carried out under a nitrogen flow in deoxygenated anhydrous CH₂Cl₂ or THF solutions of tetra-n-butylammonium hexafluorophosphate (0.1 M). Glassy carbon was used as the working electrode, a Pt wire as the counter electrode, and an Ag wire anodized with AgCl as the pseudo-reference electrode. Potentials were referenced to the ferrocenium/ferrocene (Cp₂Fe^{+/0}) couple by using ferrocene as an internal standard. Abbreviations used include singlet (s), doublet (d), doublet of doublets (dd), triplet (t), triplet of doublets (td) and unresolved multiplet (m). Mass spectral analyses were provided by the Georgia Tech Mass Spectrometry Facility. Elemental analyses were provided by Atlantic Microlab, Inc.

Unless otherwise noted, cited reagents and solvents were purchased from well-known commercial sources (such as Sigma-Aldrich of Milwaukee Wis. or Acros Organics of Geel Belgium), and were used as received without further purification.

### Example 1

### 3,3′-Dibromo-5,5′-bis-trimethylsilanyl-2,2′-bithiophene (1a)

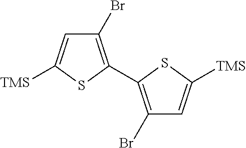 [SMILES: C[Si](C)(C)c1cc(Br)c(-c2sc([Si](C)(C)C)cc2Br)s1]

2-Bromothiophene (0.10 mol, 16.3 g) was dissolved in 200 ml of anhydrous THF and the colorless solution was cooled in acetone/dry ice bath. LDA (1.2 M in hexanes-THF, 0.10 mol, 83.3 ml) was added dropwise and clear yellow-orange solution was stirred for 1 h. Chlorotrimethylsilane (1.0 eq., 0.10 mol, 10.86 g) was added dropwise, the mixture was stirred for 1 h and clean formation of 2-bromo-5-trimethylsilylthiophene was confirmed by GC/MS analysis. LDA (1.2 M in hexanes-THF, 1.1 eq., 0.11 mol, 91.7 ml) was added dropwise, and after stirring for 0.5 h thick suspension formed. Completion of the BCHD reaction was confirmed by GC/MS analysis and CuCl₂ (1.1 eq., 0.11 mol, 14.79 g) was added in one portion. Dark green mixture was allowed to slowly warm to room temperature overnight. Hexanes and water were added (copper salts partially precipitated out) and the organic phase was carefully removed. The aqueous phase was extracted with hexanes several times and combined organic phases were dried over MgSO₄. The solvents were removed by rotary evaporation and the residue (oil with some green copper salts) was dissolved in hexanes. This solution was filtered through silica gel plug (hexanes as eluant), the solvent was removed from brownish solution and the crude product was obtained as oil, which partially solidified overnight. This crude material was purified by Kugelrohr distillation and the product was obtained as yellow oil at 175-180° C./1.0-1.2 mm Hg (this oil solidified on standing, 19.80 g, 84.5% yield). UV-vis (CH₂Cl₂) λₘₐₓ, nm 226, 266. ¹H NMR (400 MHz, CDCl₃): δ 7.20-7.10 (s, 2H); 0.40-0.30 (s, 18H); ¹³C{¹H} NMR (100 MHz, CDCl₃): δ 142.76, 136.86, 133.78, 112.78, −0.52.

### Example 2

### 3,3′-Dibromo-5,5′-bis-trimethylsilanyl-2,2′-biselenophene (2a)

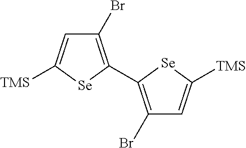 [SMILES: C[Si](C)(C)c1cc(Br)c(-c2[se]c([Si](C)(C)C)cc2Br)[se]1]

A solution of diisopropylamine (distilled from CaH₂, 48.4 mmol, 4.90 g) in anhydrous THF (20 ml) was cooled in acetone/dry ice bath and n-butyllithium (2.5 M in hexanes, 44.0 mmol, 17.6 ml) was added dropwise. The cooling bath was removed and the mixture was stirred for 0.5 h. Portion of this freshly prepared LDA (1.0 M, 20.0 mmol, 20 ml) was added dropwise to a colorless solution of 2-bromoselenophene (20.0 mmol, 4.20 g) in 100 ml of anhydrous THF (acetone/CO₂ bath). During the addition of LDA the reaction mixture changed color from colorless to yellow. The reaction mixture was stirred for 0.5 h and chlorotrimethylsilane (20.0 mmol, 2.17 g) was added dropwise. The mixture was stirred for 20 minutes and clean formation of 2-bromo-5-trimethylsilyl-selenophene was confirmed by GC/MS analysis. LDA (1.0 M, 24.0 mmol, 24 ml) was added dropwise, the reaction mixture was stirred for 0.5 min and completion of BCHD reaction was confirmed by GC/MS analysis. CuCl₂ (20.0 mmol, 2.69 g) was added in one portion, the resulting mixture was stirred for 2 hours and the cooling bath was removed. The dark yellow-brownish reaction mixture was poured into ˜50 ml of brine, diluted with ˜50 ml of hexanes and copper salts partially precipitated out. The organic phase was removed, the aqueous phase was extracted with hexanes (3×20 ml) and the combined organic phases were dried over MgSO₄. The solvents were removed by rotary evaporation, the residue was dissolved in hexanes and filtered through silica gel plug (200 ml of hexanes, then hexanes:EtOAc (50:1, 200 ml) as eluants). The solvents were removed from bright yellow solution and orange oil was purified by column chromatography to give product as a yellow solid (3.74 g, 66.6% yield). HRMS (EI) calculated for C₁₄H₂₀Br₂Se₂Si₂ 561.7801; found 561.7797. ¹H NMR (CDCl₃, 400 MHz): δ 7.46 (s, 2H), 0.33 (s, 18H); ¹³C{¹H} NMR (CDCl₃, 100 MHz): δ 145.0, 139.5 (CH), 139.4, 113.3, −0.1. Anal. Calc. for C₁₄H₂₀Br₂Se₂Si₂: C, 29.91; H, 3.59. Found: C, 30.15; H, 3.53.

### Example 3

### 3,3′5,5′-Tetrabromo-4,4′-di-n-hexyl-2,2′-bithiophene (3a)

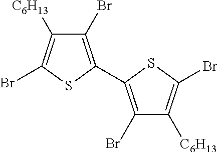

Diisopropylamine (distilled from CaH₂, 90.0 mmol, 9.11 g) was dissolved in anhydrous THF (160 ml) under nitrogen atmosphere and the resulting solution was cooled (acetone/dry ice bath). n-Butyllithium (2.5 M in hexanes, 82.5 mmol, 33.0 ml) was added dropwise, the cooling bath was removed and the mixture was stirred for half an hour. This freshly prepared solution of LDA was cooled (acetone/dry ice bath) and 2,5-dibromo-3-n-hexylthiophene (75.0 mmol, 24.46 g) was added dropwise. The bright yellow reaction mixture was stirred for 1 h and CuCl₂ (82.5 mmol, 11.09 g) was added in one portion. The mixture from yellow-orange became blue. The reaction mixture was allowed to warm slowly to room temperature overnight (without cooling bath removal). The reaction mixture was treated with water (˜70 ml) and hexanes (copper salts precipitated out). The organic phase was removed, the aqueous phase was extracted with hexanes two times and the combined organic phases were dried over MgSO₄. The solvents were removed by rotary evaporation and the crude product was obtained as brownish oil. The crude product was dissolved in hexanes and filtered through silica gel plug (˜400 ml of hexanes as eluant was used). Barely yellowish solution was collected (brown and green matter got stuck on the silica gel), the solvent was removed and the yellowish oil was dried under vacuum. Yellowish oil solidified on scratching and yellowish solid was obtained (19.74 g, 81.0%). HRMS (EI) calculated for C₂₀H₂₆Br₄S₂ 645.8209; found 645.8171. ¹H NMR (CDCl₃, 400 MHz): δ 2.64 (t, J=7.9 Hz, 2H), 1.53 (m, 2H), 1.43-1.20 (m, 6H), 0.88 (t, J=6.8 Hz, 3H); ¹³C{¹H} NMR (CDCl₃, 100 MHz): δ 141.5, 128.5, 114.6, 111.0, 31.5, 30.3, 29.0, 28.5, 22.6, 14.1. Anal. Calc. for C₂₀H₂₆Br₄S₂: C, 36.95; H, 4.03. Found: C, 37.23; H, 4.03.

### Example 4

### 4,4′-Dibromo-2,2′-bis(triisopropylsilyl)-5,5′-bithiazole (4a)

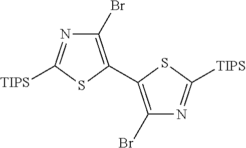

2-Triisopropylsilyl-5-bromothiazole (6.71 mmol, 2.15 g) was dissolved in 70 ml of anhydrous THF under nitrogen atmosphere and the resulting colorless solution was cooled in acetone/dry ice bath. LDA (1.5 M in hexanes-THF, 1.1 eq., 7.38 mmol, 4.9 ml) was added dropwise and the reaction mixture became bright yellow. The mixture was stirred for 15 minutes, a small aliquot was treated with hexanes-MeOH, the solvent was removed and the residue was analyzed by ¹H NMR. The completion of the BCHD reaction was confirmed and CuCl₂ (1.1 eq., 7.38 mmol, 0.99 g) was added in one portion. The reaction mixture became dark green in color. After stirring for 2 h the cooling bath was removed, the mixture was warmed to room temperature, treated with hexanes (˜70 ml) and water and copper salts precipitated out. The organic phase was removed, the aqueous phase was extracted with hexanes (3×20 ml) and combined organic phases were dried over MgSO₄. The solvent was removed by rotary evaporation and the crude product was obtained as brownish solid. This material was purified by column chromatography (200 ml of silica gel, hexanes:CH₂Cl₂ (2:1) as eluant). First several fractions containing slightly contaminated material were combined separately, the solvent was removed and yellowish solid (0.514 g) was further purified by recrystallization from ˜45 ml of EtOH. Off white crystalline material was obtained after vacuum filtration (0.412 g, 80.2% recovery). Fractions with pure material were combined separately, the solvents were removed by rotary evaporation and the yellowish solid (1.24 g) was recrystallized from ˜80 ml EtOH. Off white shiny solid was obtained after vacuum filtration (1.09 g, 87.9% recovery). Total yield of the product before recrystallization was 81.8% (1.75 g), the recovery after recrystallization was 85.6% (1.50 g). UV-vis (CH₂Cl₂) λₘₐₓ: 225, 314. HRMS (EI) calculated for C₂₄H₄₂Br₂N₂S₂Si₂ 636.0695; found 636.0669. ¹H NMR (CDCl₃, 400 MHz): δ1.48 (septet, 6H), 1.75 (d, J=7.6 Hz, 36H); ¹³C{¹H} NMR (CDCl₃, 100 MHz): δ 172.5, 130.3, 125.0, 18.4, 11.5. Anal. Calc. for C₂₄H₄₂Br₂N₂S₂Si₂C, 45.13; H, 6.63; N, 4.39. Found: C, 44.86; H, 6.53; N, 4.36.

### Example 5

### 2,2′-Difluoro-4,4′-diiodo-3,3′-bipyridine (5a)

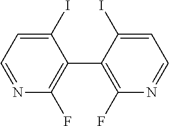 [SMILES: Fc1nccc(I)c1-c1c(I)ccnc1F]

2-Fluoro-3-iodopyridine (7.80 mmol, 1.74 g) was dissolved in 40 ml of anhydrous THF under nitrogen atmosphere and the solution was cooled in acetone/CO₂ bath. LDA (1.1 eq., 1.2 M in hexanes-THF, 8.58 mmol, 7.15 ml) was added dropwise. The reaction mixture became yellowish and after stirring for 0.5 h it was analyzed by GC/MS. A clean BCHD reaction was confirmed, the mixture was stirred for additional 0.5 h and CuCl₂ (1.1 eq., 8.58 mmol, 1.15 g) was added in one portion. The yellow reaction mixture became dark blue, then brown red (within 1-2 h) and then light greenish after warm up to room temperature. The reaction mixture was treated with hexanes and water, the organic phase was removed, and the aqueous phase was extracted with hexanes (2×˜20 ml). The combined organic phases were dried over MgSO₄ and the solvent was removed by rotary evaporation to give greenish-brownish oil which partially solidified on standing. This crude product was purified by column chromatography (200 ml of silica gel, hexanes:CH₂Cl₂ mixtures (2:1, 1:1: and then 1:2) as eluants). The solvents were removed from combined fractions and the product was obtained as off-white solid (1.04 g, 60.1%). UV-vis (CH₂Cl₂) λₘₐₓ, nm 226, 244, 268. HRMS (EI) calculated for C₁₀H₄F₂₁₂N₂ 443.8432; found 443.8417. ¹H NMR (CDCl₃, 400 MHz): δ 8.01 (d, J=5.2 Hz, 2H), 7.82 (d, J=5.2 Hz, 2H); ¹³C{¹H} NMR (CDCl₃, 100 MHz): δ 159.28 (d, J (C—F)=242.8 Hz, quaternary C), 148.5 (d, J (C—F)=15.62 Hz, CH), 132.1 (d, J (C—F)=4.6 Hz, CH), 125.0 (dd, J(C—F)=33.4 Hz, 4.1 Hz), 114.5 (d, J(C—F)=1.7 Hz). Anal. Calc. for C₁₀H₄F₂₁₂N₂: C, 27.05; H, 0.91; N, 6.31. Found: C, 27.52; H, 0.84; N, 6.19.

### Example 6

### 4,4′-Dibromo-5,5′-bis(trimethylsilyl)-2,2′-bithiazole (6a)

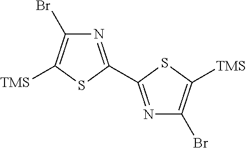 [SMILES: C[Si](C)(C)c1sc(-c2nc(Br)c([Si](C)(C)C)s2)nc1Br]

2-Bromothiazole (40.0 mmol, 6.56 g) was dissolved in 125 ml of anhydrous THF under nitrogen atmosphere, chlorotrimethylsilane (40.0 mmol, 4.34 g) was added and the resulting mixture was cooled (hexanes/N₂ bath). LDA (1.2 M in hexanes-THF, 40.0 mmol, 33.3 ml) was added dropwise and the colorless solution became yellow and then yellow-orange (−90-80° C. internal temperature). GC/MS analysis confirmed a clean formation of 2-bromo-5-trimethylsilylthiazole. The second equivalent of LDA (1.2 M in hexanes-THF, 44.0 mmol, 36.7 ml) was added dropwise (−85° C. internal temperature) and the mixture became green after addition of 5 ml of LDA. After completion of addition of LDA the dark brown reaction mixture was stirred for 10 minutes (−85-80° C. internal temperature) and analyzed by GC/MS. GC/MS analysis showed the presence of rearranged species as a major compound, CuCl₂ (40.0 mmol, 5.38 g) was added in one portion and the mixture was slowly warmed to room temperature without removal of a cooling bath. After 50 minutes the reaction mixture was analyzed by product was detected as a major compound. The reaction mixture was treated with ˜40 ml of water (copper salts partially precipitated out), organic phase was separated and the aqueous phase was extracted with hexanes several times and the dark brown organic phases were dried over MgSO₄. The solvents were removed by rotary evaporation and the crude material was obtained as brown-orange solid. This crude compound was dissolved in hexanes under heating and the cloudy solution was filtered through silica gel plug (hexanes, then hexanes:Et₂O (˜10:1) and slightly impure compound was obtained as orange solid (6.6 g, 68.0% yield). This material was further purified by recrystallization from EtOH and yellow solid was obtained after vacuum filtration (4.3 g, 65% recovery). Additional amount of material can be obtained from the mother liquor. UV-vis (CH₂Cl₂) λₘₐₓ (nm) 339, 347, 251. HRMS (EI) calculated for C₁₂H₁₈Br₂N₂S₂Si₂ 467.8817; found 467.8834. ¹H NMR (CDCl₃, 400 MHz): δ 0.45 (s, 18H); ¹³C{¹H} NMR (CDCl₃, 100 MHz): δ 163.2, 123.1, 132.0, −0.9. Anal. Calc. for C₁₂H₁₈Br₂N₂S₂Si₂: C, 30.64; H, 3.86; N, 5.96. Found: C, 30.85; H, 3.77; H, 5.69.

### Example 7

### 4,4′-Dibromo-2,2′-bis(4-n-hexyl-5-(trimethylsilyl)thiophen-2-yl)-5,5′-bithiazole (7a)

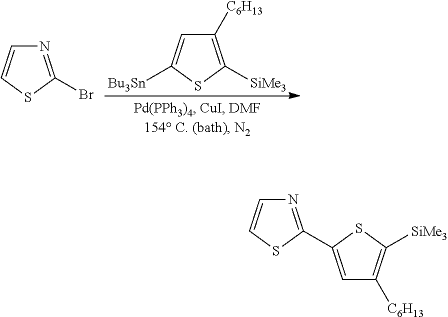

2-Bromothiazole (5.0 mmol, 0.82 g) was mixed with 2-trimethylsilyl-3-n-hexyl-5-tri-n-butylstannylthiophene (1.05 eq., 5.25 mmol, 2.78 g) in an oven-dried Schenk flask. Pd(PPh₃)₄ (0.01 mol %, 0.05 mmol, 0.058 g) and CuI (0.003 mmol, 0.025 mmol, 3.0 mg) and 10 ml of anhydrous DMF were added and the mixture was heated up to 154° C. (bath temperature). The mixture became orange and then after 15 minutes it rapidly changed to brown. TLC analysis (CH₂Cl₂ as eluant) confirmed the complete consumption of 2-bromothiazole and the mixture was cooled to room temperature. Water was added and organic phase was extracted with hexanes. Organic phase was treated with KF_{aq} and syrup-like organic phase was dried over MgSO₄ and filtered through Celite. The solvent was removed from thick solution and the residue was purified by column chromatography (100 ml of silica gel, Hexanes:CH₂Cl₂ (2:1) as eluant; note: the residue was dissolved in dichloromethane and some insoluble white solid (presumably tin salts) was left behind). The solvents were removed from combined fractions and the resulting oil was dried under vacuum (1.12 g, 69.2% yield). GC/MS: 323 at 14.99 min (exact mass calculated for C₁₆H₂₅NS₂Si 323.1198). ¹H NMR (CDCl₃, 400 MHz): δ 7.74 (d, J=3.3 Hz, 1H), 7.45 (s, 1H), 7.21 (d, J=3.3 Hz, 1H), 2.65 (m, 2H), 1.65 (m, 2H), 1.45-1.25 (m, 6H), 1.38 (m, 3H), 0.37 (s, 18H); ¹³C{¹H} NMR (CDCl₃, 100 MHz): 8161.9 (quaternary C), 151.1 (quaternary C), 143.2 (CH), 140.3 (quaternary C), 136.3 (quaternary C), 129.5 (CH), 117.8 (CH), 31.7 (CH₂), 31.8 (CH₂), 31.6 (CH₂), 31.3 (CH₂), 29.3 (CH₂), 22.5 (CH₂), 14.0 (CH₃), 0.1 (CH₃ of SiMe₃) (assignment of the quaternary, CH, CH₂ and CH₃ signals was made based on the DEPT experiment).

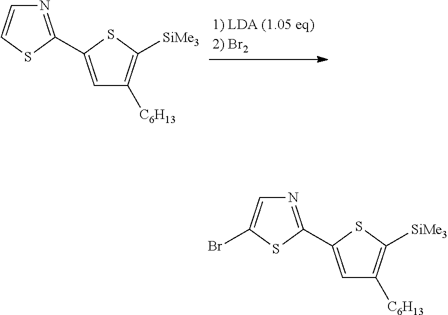

LDA was prepared from diisopropylamine (1.2 eq., 3.6 mmol, 0.36 g), n-butyllithium (2.5 M in hexanes, 3.15 mmol, 1.26 ml) and 10 ml of anhydrous THF. 2-(5-Trimethylsilyl-3-n-hexylthiophen-2-yl)-thiazole (3.0 mmol, 0.97 g) was dissolved in 20 ml of anhydrous THF in a three-necked round bottom flask equipped with magnetic stirbar, nitrogen inlet, thermometer and septum. The colorless solution was cooled in acetone/dry ice bath and freshly prepared LDA was added dropwise (−70 to −65° C. internal temperature). The light purple solution was stirred for 1 h and bromine (1.05 eq., 3.15 mmol, 0.50 g) was added dropwise. The grey reaction mixture became dark in color and then within minutes it became yellow-orange. The mixture was warmed to room temperature, treated with aqueous Na₂S₂O₃ and organic phase was separated. The aqueous phase was extracted with hexanes (3×15 ml) and combined organic phases were dried over MgSO₄. The solvents were removed by rotary evaporation and the crude product was obtained as orange oil, which was purified by column chromatography (100 ml of silica gel, hexanes:CH₂Cl₂ (3:2 as eluant). The solvent was removed from combined fractions and the yellowish oil was dried under vacuum (0.53 g, 43.9% yield). GC/MS: 401 and 403 at 17.08 min (exact mass calculated for C₁₆H₂₄BrNS₂Si 401.0303). ¹H NMR (CDCl₃, 400 MHz): δ 7.62 (s, 1H), 7.37 (s, 1H), 2.64 (t, J=8.0 Hz, 2H), 1.61 (m, 2H), 1.42-1.28 (m, 6H), 0.91 (t, J=6.7 Hz, 3H), 0.36 (s, 9H); ¹³C{¹H} NMR (CDCl₃, 100 MHz): δ 163.1 (quaternary C), 151.1 (quaternary C), 144.4 (CH), 139.7 (quaternary C), 137.1 (quaternary C), 129.6 (CH), 107.3 (quaternary C—Br), 31.7 (CH₂), 31.6 (CH₂), 31.3 (CH₂), 29.3 (CH₂), 22.6 (CH₂), 14.0 (CH₃), 0.1 (CH₃ of SiMe₃) (this material still contained ˜8% of impurity based on NMR analysis).

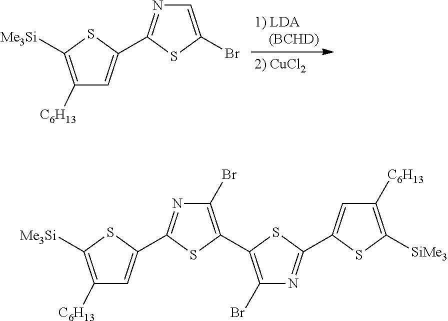

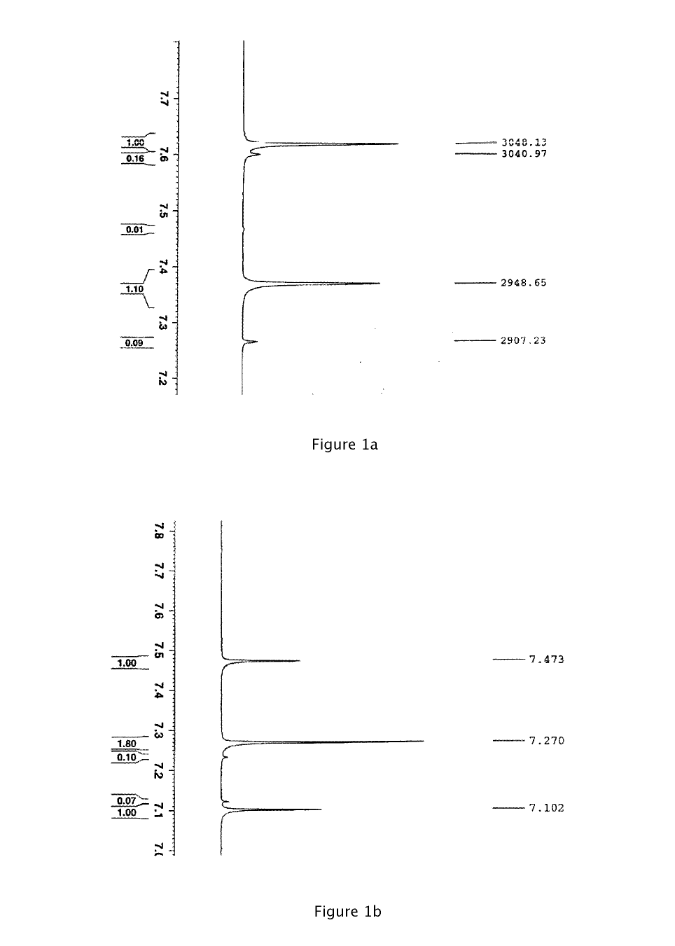

LDA (2.2 eq., 0.37 M, 6 ml) was prepared from diisopropylamine (2.4 mmol, 0.24 g), n-butyllithium (2.5 M in hexanes, 2.2 mmol, 0.9 ml) and 5 ml of anhydrous THF). 2-(5-Trimethylsilyl-3-n-hexyl-thiophen-2-yl)-5-bromothiazole (1.0 mmol, 0.40 g) was dissolved in 20 ml of anhydrous THF and the yellowish solution was cooled in acetone/dry ice bath (nitrogen atmosphere). Freshly prepared LDA (0.37 M in THF, 1.1 eq., 3 ml) was added dropwise to the bromothiazole derivative and the reaction mixture became light purple in color. The reaction mixture was stirred for 20 minutes and a small aliquot was removed for analysis as described immediately below, and treated with hexanes:MeOH. Organic solvents were removed from the analytical sample, and the residue was analyzed by GC/MS analysis and ¹H NMR. The NMR analysis of the analytical sample is shown in FIG. 1.

The completion of the BCHD reaction was confirmed by the NMR of the analytical sample, and CuCl₂ (1.1 eq., 0.148 g) was added in one portion to the remaining purple reaction mixture. After stirring for 5 minutes the color changed to yellowish-green and the mixture was slowly warmed to room temperature without cooling bath removal. Hexanes and water were added, the organic phase was removed and the aqueous phase was extracted with Et₂O (3×15-20 ml). The combined organic phases were dried over MgSO₄ and the solvents were removed by rotary evaporation to give crude product as dark yellow solid. This crude product was purified by column chromatography (50 ml of silica gel, hexanes:CH₂Cl₂ (3:2) and bright yellow-orange solid was obtained (0.27 g, 67.3%). Minor impurities were detected by the TLC analysis and material was further purified by the column chromatography (100 ml of silica gel, Hexanes:CH₂Cl₂ (35:15). The solvents were removed from combined fractions and product was obtained as yellow-orange oil which solidified on standing (0.13 g, 48% recovery, 32% yield). HRMS calculated for C₃₂H₄₆Br₂N₂S₄Si₂ 800.0449; found 800.0420. ¹H NMR (CDCl₃, 400 MHz): δ 7.53 (s, 2H), 2.66 (t, J=8.0 Hz, 4H), 1.62 (m, 4H), 1.45-1.30 (m, 12H) 0.98 (t, J=6.9 Hz, 6H), 0.38 (s, 18H); ¹³C{¹H} NMR (CDCl₃, 100 MHz): δ 162.1 (quaternary C), 151.4 (quaternary C), 139.0 (quaternary C), 138.5 (quaternary C), 130.5 (CH), 127.6 (quaternary C), 1210 (quaternary C), 31.7 (CH₂), 31.6 (CH₂), 31.3 (CH₂), 29.3 (CH₂), 22.6 (CH₂), 14.1 (CH₃), 0.1 (CH₃) (assignment of the quaternary, CH, CH₂ and CH₃ signals was made based on the DEPT experiment). Elemental analysis calculated for C₃₂H₄₆Br₂N₂S₄Si₂: C, 47.87; H, 5.77; N, 3.49. Found: C, 47.72; H, 5.77; N, 3.47.

### Example 9

### 2,6-Bis-trimethylsilanyl-cyclopenta[2,1-b;3,4-b]dithiophen-4-one (1b)

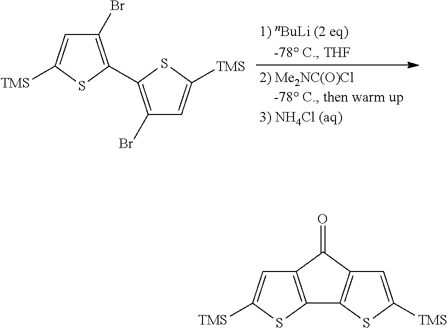 [SMILES: C[Si](C)(C)c1cc(Br)c(-c2sc([Si](C)(C)C)cc2Br)s1 ; C[Si](C)(C)c1cc2c(s1)-c1sc([Si](C)(C)C)cc1C2=O]

3,3′-Dibromo-5,5′-bis-trimethylsilanyl-2,2′-bithiophene (1a) (25.62 mmol, 12.00 g) was dissolved in anhydrous THF (100 ml) under nitrogen atmosphere and the colorless solution was cooled in acetone/dry ice bath. n-Butyllithium (2.5 M in hexanes, 2 eq., 51.24 mmol, 20.5 ml) was added and the colorless reaction mixture became bright yellow in color. After stirring for 15 minutes N,N-dimethylcarbamoyl chloride (1 eq., 25.62 mmol, 2.76 g) in 20 ml of anhydrous THF was added dropwise and the deep-yellow mixture was allowed to warm up. The mixture was stirred for 2.5 h and NH₄Cl (10 g) in water (75 ml) was added carefully, and the dark orange-brown solution became intense red (almost black red). The dark red organic phase was removed, the aqueous phase was extracted with hexanes several times, and the combined organic extracts were dried over MgSO₄. The solvents were removed by rotary evaporation and the crude product (11.0 g) was purified by Kugelrohr distillation. Bright red oil was collected at 190° C./0.25 mm Hg and some brown-orange matter was left in the original distillation flask. The product was obtained as bright red solid in 85.8% yield (7.40 g). Analytically pure compound was obtained after column chromatography purification (silica gel, hexanes as eluant to remove minor impurities, then hexanes:EtOAc (30:1) as eluant for the product). IR (KBr, cm⁻¹): 2955, 2896, 1702, 1466, 1420, 1355, 1248, 1168, 1020, 961, 838, 753, 695, 620, 556, 487. UV-vis (CH₂Cl₂) λₘₐₓ (nm) 273, 282, 494. HRMS (EI) calculated for C₁₅H₂₀OS₂Si₂ 336.0494; found 336.0490. ¹H NMR (400 MHz, CDCl₃): δ 7.07 (s, 2H, two Th-H), 0.32 (s, 18H, two SiMe₃). ¹³C{¹H} NMR (400 MHz, CDCl₃): δ 183.1, 154.3, 144.9, 144.1, 127.9, −0.3. Anal. Calc. for C₁₅H₂₀OS₂Si₂: C, 53.52; H, 5.99. Found: C, 53.39; H, 6.11.

### Example 10

### 2,6-Bis(trimethylsilyl)-4-(3,4,5-tris(dodecyloxy)phenyl)-4H-dithieno[3,2-b:2′,3′-d]pyrrole (1c)

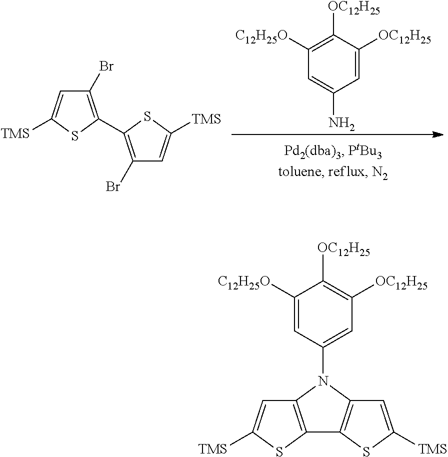

Catalyst Pd₂(dba)₃ (0.125 mmol, 0.115 g, where dba is tris(dibenzylideneacetone)dipalladium(0)), tri-^{t}butylphosphine (10 wt % in hexanes, 0.625 mmol, 1.26 ml) and 25 ml of anhydrous toluene were stirred under nitrogen atmosphere for 20 minutes (dark purple solution) and 3,3′-dibromo-5,5′-bis-trimethylsilanyl-2,2′-bithiophene (1a) (2.5 mmol, 1.17 g), 3,4,5-tris(dodecyloxy)aniline (2.625 mmol, 1.695 g) and ^{t}BuONa (11.5 mmol, 1.09 g) were added (nitrogen atmosphere). The resulting dark brown-orange mixture was refluxed for 0.5 h, analyzed by TLC (hexanes as eluant) and consumption of the starting dibromide 1a was confirmed and a new more polar product was detected. The reaction mixture was cooled to room temperature and treated with ˜15 ml of water. The brown organic phase was separated and the aqueous phase was extracted with hexanes (2×˜15 ml). The combined organic phases were dried over MgSO₄, the solvents were removed by rotary evaporation and the crude product was purified by column chromatography (150 ml of silica gel, hexanes and then hexanes:CH₂Cl₂ (2:1) as eluants). Combined fractions were subjected to rotary evaporation and the residue was dried under vacuum. The product was obtained as very thick yellowish oil (52.9% yield). UV-vis (CH₂Cl₂) λₘₐₓ (nm) 266, 315, 329. MS (MALDI) calculated for C₅₆H₉₇NO₃S₂Si₂ 951.6448; found 951.6. ¹H NMR (400 MHz, CDCl₃): δ 7.21 (s, 2H), 6.78 (s, 2H), 4.02 (m, 6H), 1.84 (m, 6H), 1.51 (m, 6H), 1.45-1.15 (m, 48H), 0.90 (m, 6H), 0.37 (s, 18H); ¹³C{¹H} NMR (400 MHz, CDCl₃): δ 153.7 (quaternary C), 147.0 (quaternary C), 139.3 (quaternary C), 136.5 (quaternary C), 135.4 (quaternary C), 121.6 (quaternary C), 117.9 (CH), 102.2 (CH), 73.7 (CH₂), 69.3 (CH₂), 31.9 (4) (CH₂), 31.9 (2) (CH₂), 30.4 (CH₂), 29.8 (CH₂), 29.8 (CH₂), 29.7 (CH₂), 29.7 (CH₂), 29.4 (CH₂), 29.4 (CH₂), 29.3 (CH₂), 26.2 (CH₂), 26.1 (CH₂), 22.7 (CH₂), 14.1 (CH₃), −0.1 (CH₃) (assignment was made based on DEPT experiment; several CH₂ carbons in the alkyl chains are missing presumably due to overlap). Anal. Calc. for C₅₆H₉₇NO₃S₂Si₂: C, 70.60; H, 10.26; N, 1.47. Found: C, 70.59; H, 10.52; N, 1.55.

### Example 11

### 2,7-Bis-trimethylsilyl-benzo[2,1-b:3,4-b′]dithiophene-4,5-dione (1d)

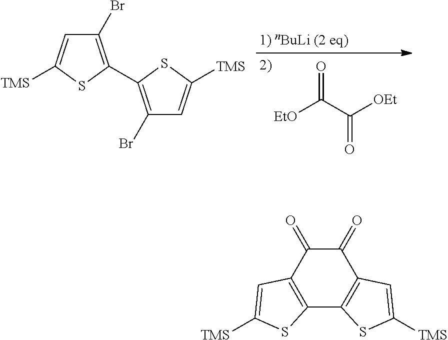 [SMILES: C[Si](C)(C)c1cc(Br)c(-c2sc([Si](C)(C)C)cc2Br)s1 ; CCOC(=O)C(=O)OCC ; C[Si](C)(C)c1cc2c(s1)-c1sc([Si](C)(C)C)cc1C(=O)C2=O]

3,3′-Dibromo-5,5′-bis-trimethylsilyl-2,2′-dithiophene (1a) (60.0 mmol, 28.11 g) was dissolved in anhydrous THF (240 mL), the solution was cooled in acetone/dry ice bath and n-butyllithium (2.87 M in hexanes, 2 eq., 120.0 mmol, 41.8 mL (caution! added in several portions with volume less than 20 mL) was added dropwise. The yellow-orange solution was stirred for 0.5 h and then transferred via cannula into a solution of diethyl oxalate (1.3 eq., 78.0 mmol, 11.40 g) in 200 mL of anhydrous THF (cooled in acetone/dry ice bath). After completion of the addition of the di-lithiated species to the diethyl oxalate, the orange-reddish mixture was stirred for 45 minutes and transferred via cannula into a solution of aqueous NH₄Cl. The dark red organic phase was separated, the aqueous phase was extracted with hexanes, and the combined organic phases were dried over MgSO₄. The solvents were removed by rotary evaporation and the crude product was heated to reflux with ˜500 ml of ethanol, cooled to room temperature, and dark-red needles were separated by the vacuum filtration (16.3 g, 76.7% yield). The mother liquor was subjected to rotary evaporation and the residue was recrystallized from ethanol to give additional amount of product (0.7 g, total yield 17.0 g, 79.9%). ¹H NMR (CDCl₃, 400 MHz): δ 7.60 (s, 2H), 0.36 (s, 18H, 6CH₃); ¹³C{¹H} NMR (CDCl₃, 100 MHz): δ 175.2 (quaternary C), 148.3 (quaternary C), 142.5 (quaternary C), 135.8 (quaternary C), 134.4 (CH), −0.44 (CH₃). HRMS (EI) calculated for C₁₆H₂₀O₂S₂Si₂ 364.0443; found 364.0469. Anal. Calc. for C₁₆H₂₀O₂S₂Si₂: C, 52.70; H, 5.53. Found: C, 52.70; H, 5.36.

Alternatively this compound was prepared using N,N-dimethyl-piperazine-2,3-dione instead of diethyl oxalate. 3,3′-Dibromo-5,5′-bis-trimethylsilanyl-2,2′-bithiophene (6.5 mmol, 3.045 g) was dissolved in anhydrous THF (100 ml), the colorless solution was cooled in acetone/CO₂ bath and n-BuLi (2.5M in hexanes, 13.0 mmol, 5.2 ml) was added dropwise. Bright yellow solution was stirred for 25 minutes and N,N-dimethyl-piperazine-2,3-dione (6.5 mmol, 0.924 g) was added in one portion. The flask was placed into ice-water bath, and the mixture was stirred for 17 h. The orange-yellow mixture was treated with aqueous NH₄Cl and the dark red organic phase was separated. The aqueous phase was extracted with Et₂O (2×15 ml) and combined organic phases were dried over MgSO₄. The solvent was removed by rotary evaporation and the residue was purified by column chromatography (250 ml of silica gel, hexanes:CH₂Cl₂:EtOAc (200:100:3 and then 200:100:6) as eluants. Combined fractions were subjected to rotary evaporation and material was obtained as red tiny needles (0.98 g, 41.4% yield). This material was recrystallized from ˜40 ml of EtOH, and dark red needles were collected by vacuum filtration (0.94 g, 95.9% recovery).

### Example 12

### 2,6-Bis(trimethylsilyl)-4-(3,4,5-tris(dodecyloxy)phenyl)-4H-diselenopheno[3,2-b:2′,3′-d]pyrrole (2b)

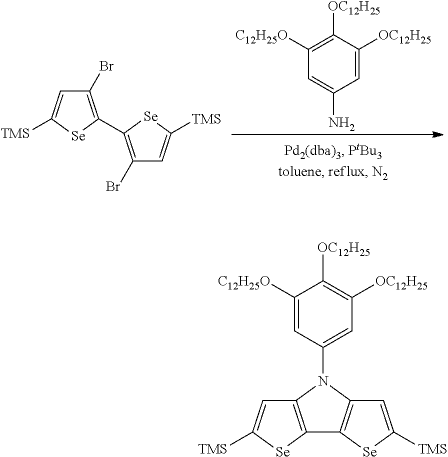

Catalyst Pd₂(dba)₃ (0.319 mmol, 0.292 mg), tri-^{t}butylphosphine (10 wt % in hexanes, 1.60 mmol, 3.23 ml) and 75 ml of anhydrous toluene were stirred for 20 minutes (purple solution) under nitrogen atmosphere and 5,5′-trimethylsilyl-3,3′-dibromo-2,2′-biselenophene (2a) (6.386 mmol, 3.59 g), 3,4,5-tris(dodecyloxy)aniline (6.70 mmol, 4.33 g) and ^{t}BuONa (29.38 mmol, 2.79 g) were added. The resulting dark brown-orange mixture was refluxed for 1 h, analyzed by TLC (hexanes as eluant) and consumption of dibromide 2a was confirmed. The brown mixture was cooled to room temperature, treated with water (˜20 ml) and brown organic phase was removed. The aqueous phase was extracted with hexanes (2×20 ml) and combined organic phases were dried over MgSO₄. The solvents were removed by rotary evaporation and the crude product was obtained as brown oil. This material was purified by the column chromatography (550 ml of silica gel, hexanes (700 ml) and then hexanes:CH₂Cl₂ (2:1) as eluants). The solvent was removed by rotary evaporation and purified material was obtained as yellow oil (it typically solidifies on standing during the storage in refrigerator). MS (MALDI) calculated for C₅₆H₉₇NO₃Se₂Si₂ 1047.5337, found 1047.54. ¹H NMR (CDCl₃, 400 MHz): δ 7.49 (s, 2H), 6.73 (s, 2H), 4.05 (t, J=6.6 Hz, 2H), 3.99 (t, J=6.5 Hz, 4H), 1.84 (m, 6H), 1.49 (m, 6H), 1.28 (m, 48H), 0.89 (m, 9H), 0.35 (s, 18H); ¹³C{¹H} NMR (CDCl₃, 100 MHz): δ 153.6, 147.6, 145.3, 136.9, 135.3, 122.7, 121.2, 103.27, 73.6, 69.3, 32.0, 31.9, 31.6, 30.4, 29.8, 29.7, 29.7, 29.4, 29.4, 29.3, 26.2, 26.1, 22.68, 22.7, 14.1, 0.3 (several CH₂ signals of alkyl groups are missing due to overlap). Anal. Calc. For C₅₆H₉₇NO₃Se₂Si₂: C, 64.27; H, 9.34; N, 1.34. Found: C, 64.38; H, 9.30; N, 1.37.

### Example 13

### 2,6-Bis-trimethylsilanyl-3,5-di-n-hexyl-cyclopenta[2,1-b;3,4-b′]dithiophen-4-one (3b)

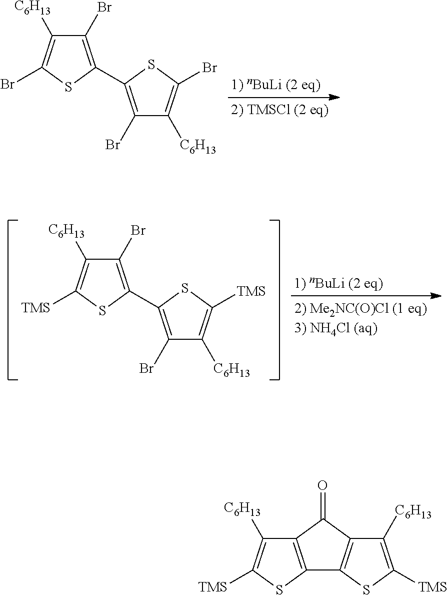

3,3′,5,5′-Tetrabromo-4,4′-di-n-hexyl-2,2′-bithiophene (23.0 mmol, 14.95 g) (3b) was dissolved in 200 ml of anhydrous THF, the solution was cooled in acetone/dry ice bath and n-butyllithium (2.5 M in hexanes, 46.0 mmol, 18.4 ml) was added dropwise (−70-65° C.). During the addition of n-BuLi the light yellowish reaction mixture became darker in color (yellow-orange), but when ˜1.5 ml of n-butyllithium was still in the syringe, the mixture became lighter yellow. The reaction mixture was stirred for 0.5 h and chlorotrimethylsilane (46.0 mmol, 5.00 g) was added dropwise (exothermic reaction), stirred for 20 min and analyzed by GC/MS. Clean formation of 3,3′-dibromo-4,4′-dihexyl-5,5′-bis-trimethylsilyl-2,2′-bithiophene was confirmed and n-butyllithium (2.5 M in hexanes, 46.0 mmol, 18.4 ml) was added dropwise (−70 to −68° C. internal temperature). The reaction mixture was analyzed by GC/MS after 5 minutes of stirring and clean lithiation was confirmed. N,N-Dimethylcarbamoyl chloride (23.0 mmol, 2.47 g) in 10 ml of anhydrous THF was added dropwise and the mixture became darker yellow in color. The reaction flask was partially removed from the cooling bath and the mixture was warmed to −40-30° C. After 40 minutes of stirring the mixture was analyzed by TLC (hexanes:EtOAc (20:1) and the product was detected as a major material. GC/MS analysis showed the presence of three species: de-brominated material (A, 17.4%), desired product (3b, 44.7%) and non-eliminated intermediate (B, 37.9%).

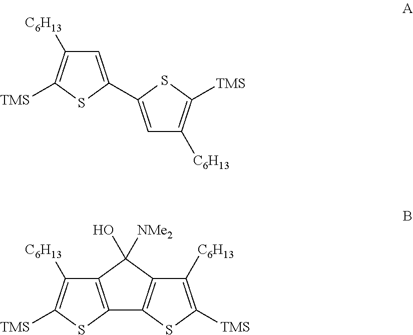

The mixture was stirred for 1.5 h, treated with NH₄Cl (12 g in 50 ml of water) (˜−30° C. internal temperature), warmed to room temperature and the dark red organic phase was separated. The aqueous phase was extracted with hexanes and combined organic phases were dried over MgSO₄. The solvent was removed by rotary evaporation and the crude product was obtained as thick red oil. This material was purified by column chromatography (200 ml of silica gel, hexanes as eluant). Fractions with a pure material were combined, the solvent was removed and the product was dried under vacuum (3.72 g). Fractions with slightly contaminated material were combined separately and further purified by column chromatography to give dark red oil, which solidified on standing (2.73 g). Total yield of the pure material was 6.45 g (55.6% yield). UV-vis (CH₂Cl₂) λₘₐₓ: 278, 287, 499. HRMS (EI) calculated for C₂₇H₄₄OS₂Si₂ 504.2370; found 504.2362. ¹H NMR (CDCl₃, 400 MHz): δ 2.63 (m, 4H), 1.55 (m, 4H), 1.40-1.25 (m, 12H), 0.87 (t, J=6.8 Hz, 6H), 0.31 (s, 18H); ¹³C{¹H} NMR (CDCl₃, 100 MHz): δ 184.5, 153.1, 147.4, 143.5, 137.1, 31.7, 31.1, 29.7, 29.3, 22.7, 14.1, 0.4. Anal. Calc. for C₂₇H₄₄OS₂Si₂: C, 64.22; H, 8.78. Found: C, 64.22; H, 8.94.

### Example 14

### 2,7-Bis-trimethylsilyl-2,6-di-n-hexyl-benzo[2,1-b:3,4-b′]dithiophene-4,5-dione (1d)

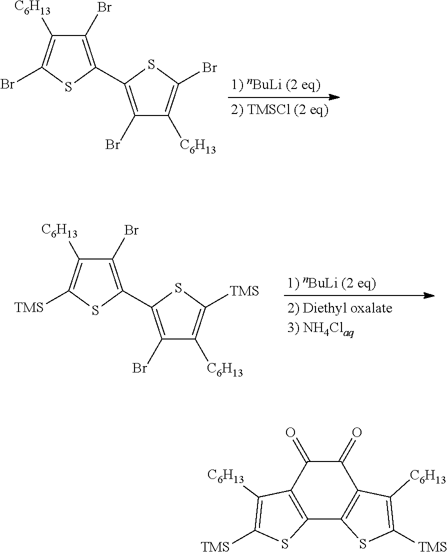

3,3′,5,5′-Tetrabromo-4,4′-di-n-hexyl-2,2′-bithiophene (3.076 mmol, 2.00 g) (3b) was dissolved in 60 ml of anhydrous THF under nitrogen atmosphere, the solution was cooled in acetone/dry ice bath and n-butyllithium (2.5 M in hexanes, 6.15 mmol, 2.5 ml) was added dropwise to the yellowish solution The reaction mixture was stirred for 15 minutes and chlorotrimethylsilane (6.15 mmol, 0.67 g) was added dropwise. The mixture was stirred for 15 minutes and n-butyllithium (2.5 M in hexanes, 6.15 mmol, 2.5 ml) was added dropwise. The reaction mixture was stirred for 0.5 h and the yellow solution was transferred via cannula to a solution of diethyl oxalate (4.24 mmol, 0.62 g) in 60 ml of THF cooled in acetone/dry ice bath. The dark yellow-brown reaction mixture was stirred for 0.5 h and transferred via cannula to an aqueous solution of NH₄Cl (13 g in 50 ml of water). Dark red organic phase was separated, the organic phase was dried over MgSO₄ and the solvents were removed by rotary evaporation to give crude product as red thick oil. This material was purified by column chromatography (250 ml of silica gel, hexanes:CH₂Cl₂ (3:2) to pack the column, hexanes to elute byproducts and then hexanes:CH₂Cl₂ (3:2) to elute the product). Solvents were removed from combined fractions (red) to give dark red oil which was dried under vacuum (oil solidified on standing, 0.56 g, 34.1%). HRMS (EI) calculated for C₂₈H₄₄O₂S₂Si₂ 532.2321; found 532.2325. ¹H NMR (CDCl₃, 400 MHz): δ 2.89 (poorly resolved t, 4H), 1.47 (m, 8H), 1.33 (m, 8H), 0.90 (poorly resolved t, 6H), 0.39 (s, 18H); ¹³C{¹H} NMR (CDCl₃, 100 MHz): δ 175.8 (quaternary C(O), 154.0 (quaternary C), 149.1 (quaternary C), 135.0 (quaternary C), 133.3 (quaternary C), 31.6 (CH₂), 30.9 (CH₂), 30.8 (CH₂), 29.8 (CH₂), 22.7 (CH₂), 14.1 (CH₃), 0.2 (CH₃) (the assignment of the carbon signals was made based on the DEPT experiment). Anal. Calc. for C₂₈H₄₄O₂S₂Si₂: C, 63.10; H, 8.32. Found: C, 62.89; H, 8.40.

### Example 15

### 2,6-Bis-trimethylsilanyl-cyclopenta[2,1-b;3,4-b]dithiazole-4-one (4b)

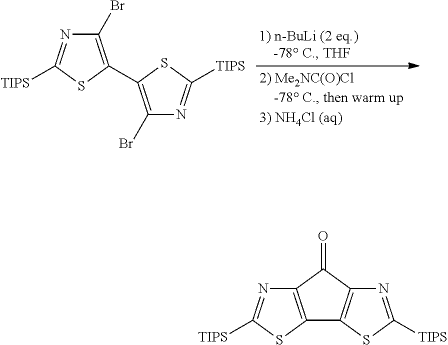

4,4′-Dibromo-2,2′-bis(triisopropylsilyl)-5,5′-bithiazole (4a) (2.0 mmol, 1.277 g) was dissolved in 80 ml of anhydrous THF under nitrogen atmosphere, the resulting colorless solution was cooled in acetone/dry ice bath and n-butyllithium (2.5 M in hexanes, 4.0 mmol, 1.6 ml) was added dropwise. The yellow solution was stirred for 20 minutes, and N,N-dimethylcarbamoyl chloride (2.0 mmol, 0.215 g) in 1 ml of anhydrous THF was added dropwise. The reaction flask was partially removed from the cooling bath, the yellow-orange mixture was stirred for 1 h, and treated with aqueous NH₄Cl. The red organic phase was removed, the aqueous phase was extracted with hexanes and combined organic phases were dried over MgSO₄. The solvents were removed by rotary evaporation and the red residue was purified by column chromatography (150 ml of silica gel, CH₂Cl₂ as eluant). The solvent was removed from combined fractions and red solid was obtained (0.392 g, 38.8% yield). UV-vis (CH₂Cl₂) λₘₐₓ: 267, 309, 492. HRMS (EI) calculated for C₂₅H₄₂N₂OS₂Si₂ 506.2277; found 506.2239. ₁H NMR (CDCl₃, 400 MHz): δ 1.46 (septet, J=7.4 Hz, 6H), 1.15 (d, J=7.5 Hz, 36H); ¹³C{³H} NMR (CDCl₃, 100 MHz): δ 179.0, 174.0, 158.2, 145.3, 18.4, 11.6. Anal. Calc. for C₂₅H₄₂N₂OS₂Si₂: C, 59.23; H, 8.35; N, 5.53. Found: C, 59.43; H, 8.44; N, 5.55.

### Example 16

### 2,7-Bis-trimethylsilyl-benzo[2,1-b:3,4-b′]dithizole-4,5-dione (4c)

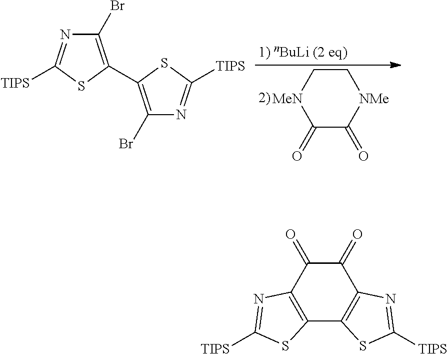

4,4′-Dibromo-2,2′-bis(triisopropylsilyl)-5,5′-bithiazole (4a) (1.5 mmol, 0.958 g) was dissolved in 75 ml of anhydrous THF under nitrogen atmosphere and the colorless solution was cooled in acetone/dry ice bath. n-Butyllithium (2.5 M in hexanes, 3.0 mmol) was added dropwise and the mixture became bright yellow. N,N-Dimethyl-piperazine-2,3-dione (1.5 mmol, 0.213 g) was added in one portion and the flask with suspension was placed into a water-ice bath. The mixture was stirred overnight, and orange-reddish solution was treated with NH₄Cl. The mixture became very dark in color and then orange-red. The organic phase was separated, the aqueous phase was extracted with hexanes and combined organic phases were dried over MgSO₄. The solvents were removed by rotary evaporation and the residue was purified by column chromatography (150 ml of silica gel, hexanes:CH₂Cl₂ (2:1, 1:1) as eluants). First two fractions with the product were kept separately and pure (by TLC) material was obtained (few mg). Fractions with slightly contaminated material were combined separately, the solvents were removed by rotary evaporation and the product was obtained as orange-red solid (0.21 g, 26.1% yield). HRMS (EI) calculated for C₂₆H₄₂N₂O₂S₂Si₂ 534.2226; found 534.2241. ¹H NMR (CDCl₃, 400 MHz): δ 1.51 (septet, J=7.5 Hz, 6H), 1.18 (d, J=7.5 Hz, 36H); ¹³C NMR (CDCl₃, 100 MHz): δ 174.0, 172.4, 149.8, 140.6, 18.4, 11.6. Anal. Calc. for C₂₆H₄₂N₂O₂S₂Si₂: C, 58.38; H, 7.91; N, 5.24. Found: C, 58.51; H, 7.98; N, 5.16.

### Example 17

### 2,6-Di-iodo-cyclopenta[2,1-b;3,4-b′]dithiophen-4-one

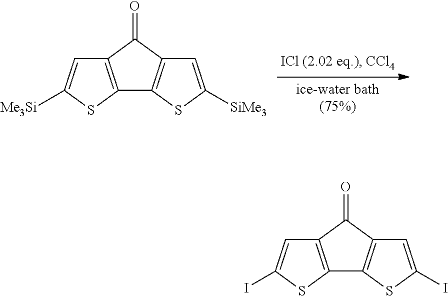 [SMILES: C[Si](C)(C)c1cc2c(s1)-c1sc([Si](C)(C)C)cc1C2=O ; O=C1c2cc(I)sc2-c2sc(I)cc21]

2,6-Bis-trimethylsilanyl-cyclopenta[2,1-b;3,4-b′]dithiophen-4-one (3.00 mmol, 1.01 g) was dissolved in 20 ml of CCl₄, a very dark red solution was cooled in ice-water bath and iodine monochloride (2.02 eq., 6.06 mmol, 0.98 g) in 10 ml of CH₂Cl₂ was added dropwise. The mixture changed color to dark purple. The cooling bath was removed, and the mixture was stirred for an hour and precipitation was observed. Water (50 ml) and several crystals of Na₂S₂O₃ were added, the bottom layer was separated, and the purple solution was dried over MgSO₄. The solvent was removed by rotary evaporation and the residue was dissolved in toluene-hexanes mixture under heating. The solution was cooled in ice-water bath and product was isolated as purple solid with some shine (0.65 g, 48.9% yield). The filter with MgSO₄ was thoroughly washed with CHCl₃, the purple solution was washed with aqueous Na₂S₂O₃, and the solvent was removed by rotary evaporation. The residue was heated with ˜30 ml of EtOAc, and the very dark solution was cooled to room temperature and then in ice-water bath. Additional amount of purple solid was obtained (0.35 g). The total amount of the product is 75.2% (1.00 g). HRMS calculated for C₉H₂I₂OS₂ 443.7636; found 443.7644. UV-vis (THF) λₘₐₓ: 207, 284, 518 (weak). ¹H NMR (THF-d8, 400 MHz): δ 7.20 (s, 2H); ¹³C{¹H} NMR (THF-d8, 100 MHz): δ 179.7, 154.5, 142.7, 131.2, 77.7. Anal. Calc. for C₉H₂I₂OS₂: C, 24.34; H, 0.45. Found: C, 24.74; H, 0.43.

### Example 18

### 2,7-Dibromo-benzo[2,1-b:3,4-b′]dithiophene-4,5-dione

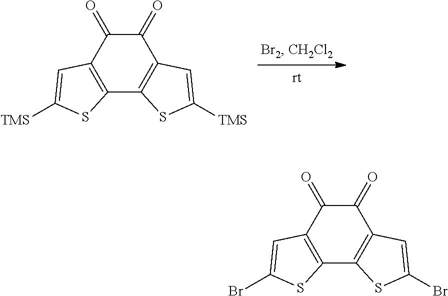 [SMILES: C[Si](C)(C)c1cc2c(s1)-c1sc([Si](C)(C)C)cc1C(=O)C2=O ; O=C1C(=O)c2cc(Br)sc2-c2sc(Br)cc21]

2,7-Bis-trimethylsilyl-benzo[2,1-b:3,4-b′]dithiophene-4,5-dione (4.0 mmol, 1.459 g) was dissolved in dichloromethane (40 ml) and bromine (2.2 eq., 8.8 mmol, 1.41 g) was added dropwise to a red-black solution. The reaction mixture became purple-black. The reaction mixture was analyzed by TLC (CH₂Cl₂ as eluant), and a new product and a minor impurity was detected. Additional amount of bromine (0.33 g) was added, the mixture was stirred for 0.5 h and treated with 10 ml of aqueous Na₂S₂O₃. The organic solvent was removed by rotary evaporation and the crude product was separated by vacuum filtration (1.95 g, 128% crude yield, slightly wet). This crude material was purified by column chromatography (300 ml of silica gel, CH₂Cl₂ as eluant). The solvent was removed from the combined fractions 3-12 and the black shiny microcrystalline material was obtained (0.90 g, 59.5% yield). The heavily stained column was eluted with chloroform, the solvent was removed from the combined fractions and additional amount of black microcrystalline solid was obtained (0.52 g, 34.4% yield). HRMS (EI) calculated for C₁₀H₂Br₂O₂S₂ 375.7863; found 375.7869. ¹H NMR (CDCl₃, 400 MHz): δ 7.47 (s, 2H); ¹³C{¹H} NMR (CDCl₃, 100 MHz): δ 172.5 (quaternary C), 143.6 (quaternary C), 135.4 (quaternary C), 130.1 (CH), 114.7 (quaternary C—Br) (assignment of the quaternary and CH signals was made based on the DEPT experiment). Anal. Calc. for C₁₀H₂Br₂O₂S₂: C, 31.77; H, 0.53. Found: C, 32.06; H, 0.40.

### Example 19

### 2,7-Dibromo-benzo[2,1-b:3,4-b′]dithiophene-4,5-dione

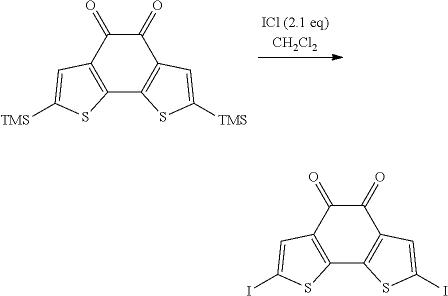 [SMILES: C[Si](C)(C)c1cc2c(s1)-c1sc([Si](C)(C)C)cc1C(=O)C2=O ; O=C1C(=O)c2cc(I)sc2-c2sc(I)cc21]

2,7-Bis-trimethylsilyl-benzo[2,1-b:3,4-b′]dithiophene-4,5-dione (2.58 mmol, 0.94 g) was dissolved in 25 ml of CH₂Cl₂ and iodine monochloride (2.1 eq., 5.41 mmol, 0.88 g) in 10 ml of CH₂Cl₂ was added dropwise to a dark red solution. The reaction mixture became purple in color and precipitate was observed. The mixture was stirred for ˜2 h at room temperature, treated with hexanes (˜30 ml) and brown-black solid was separated by vacuum filtration (1.21 g, 99.3% crude yield). This material was purified by column chromatography using hot CHCl₃ to apply the material and then CHCl₃:EtOAc (150:1) to elute the product. Fractions with pure compound were combined separately and black shiny solid was obtained after removal of the solvents (0.70 g). Fractions with slightly contaminated material were combined separately and black shiny solid was obtained after solvents removal (0.466 g). ¹H NMR (THF-d8, 400 MHz): δ 7.64 (s, 2H); ¹³C{¹H} NMR (THF-d8, 100 MHz): δ 172.6 (quaternary C), 147.0 (quaternary C), 138.4 (quaternary C), 137.4 (CH), 77.2 (quaternary C—I) (assignment of the quaternary and CH signals was made based on the DEPT experiment). HRMS (EI) calculated for C₁₀H₂I₂O₂S 471.7586; found 471.7608. Anal. Calc. for C₁₀H₂I₂O₂S₂: C, 25.44; H, 0.43. Found: C, 23.91; H, 0.54 (the TGA and NMR analysis confirmed the presence of CHCl₃, and this elemental analysis is in agreement with a material which has 1:1 ratio of diiodide and chloroform). Material was also recrystallized from toluene to potentially avoid co-crystallization with the solvent which observed for chloroform, but NMR and TGA analysis of the sample showed the presence of toluene in a sample (3.7% by TGA).

### Example 20

### 3,6-Di-n-hexyl-2,7-dibromo-benzo[2,1-b:3,4-b′]dithiophene-4,5-dione

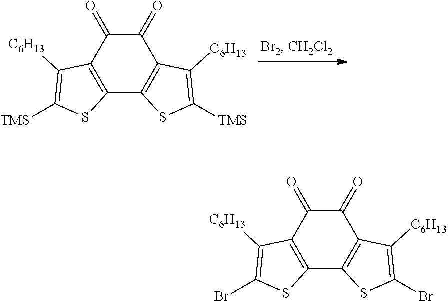

3,6-Di-n-hexyl-2,7-bis-trimethylsilyl-benzo[2,1-b:3,4-b′]dithiophene-4,5-dione (0.70 mmol, 0.37 g) was dissolved in dichloromethane (20 ml) and bromine (2.2 eq., 1.54 mmol, 0.25 g) was added dropwise to a red-purple solution. The dark purple mixture was stirred for 0.5 h and aqueous Na₂S₂O₃ was added. The organic phase was removed, dried over MgSO₄ and the solvent was partially removed by rotary evaporation. Purple solution was column chromatographed (250 ml of silica gel, hexanes:CH₂Cl₂ (1:1) to pack the column, hexanes to elute the byproduct, then hexanes:CH₂Cl₂ (1:1) to elute the product). Several fractions with slightly contaminated product was further purified by recrystallization from 2-PrOH and material was obtained as purple solid (0.163 g). Fractions with pure material were subjected to rotary evaporation and the residue was recrystallized from 2-PrOH to give purple solid (0.078 g). Total yield of the product is 63.2% (0.242 g). HRMS (EI) calculated for C₂₂H₂₆Br₂O₂S₂ 543.9741; found 543.9722. ¹H NMR (CDCl₃, 400 MHz): δ 2.88 (t, J=7.6 Hz, 4H), 1.51 (m, 4H), 1.38 (m, 4H), 1.32 (m, 8H), 0.91 (t, J=6.9 Hz, 6H); ¹³C{¹H} NMR (CDCl₃, 100 MHz): δ 173.5 (quaternary C), 145.5 (quaternary C), 144.1 (quaternary C), 131.8 (CH), 111.7 (quaternary C—Br), 31.5 (CH₂), 29.1 (CH₂), 28.7 (CH₂), 28.5 (CH₂), 22.6 (CH₂), 14.1 (CH₃) (assignment of the carbon signals was made based on the DEPT experiment). Anal. Calc. for C₂₂H₂₆Br₂O₂S₂: C, 48.36; H, 4.80. Found: C, 48.46; H, 4.81.

### Example 21

### 3,6-Di-n-hexyl-2,7-di-iodo-benzo[2,1-b:3,4-b′]dithiophene-4,5-dione

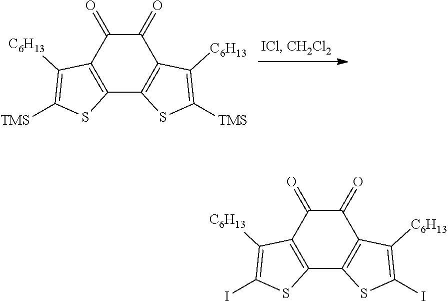

3,6-Di-n-hexyl-2,7-bis-trimethylsilyl-benzo[2,1-b:3,4-b′]dithiophene-4,5-dione (0.20 mmol, 0.107 g) was dissolved in dichloromethane (10 ml) and iodine monochloride (2.1 eq., 0.42 mmol, 0.068 g) was added dropwise to a dark red-purple solution. The purple mixture was stirred for 20 minutes and aqueous Na₂S₂O₃ was added. The purple organic phase was removed, dried over MgSO₄ and the solvent was removed by rotary evaporation. Crude product was purified by column chromatography (30 ml of silica gel, hexanes:CH₂Cl₂ (2:1) as eluant). The combined fractions were subjected to rotary evaporation and the residue was purified from 2-PrOH (˜10 ml). Material was obtained as purple solid in 55.9% yield (0.0716 mg). HRMS (EI) calculated for C₂₂H₂₆I₂O₂S₂ 639.9464; found 639.9468. ¹H NMR (CDCl₃, 400 MHz): δ 2.86 (t, J=7.4 Hz, 4H), 1.44 (m, 8H), 1.23 (m, 8H), 0.92 (t, J=7.0 Hz, 6H); ¹³C{¹H} NMR (CDCl₃, 100 MHz): δ 173.1 (quaternary C), 145.5 (quaternary C), 148.8 (quaternary C), 131.3 (CH), 78.0 (quaternary C—I), 31.5 (CH₂), 31.2 (CH₂), 29.2 (CH₂), 28.9 (CH₂), 22.6 (CH₂), 14.10 (CH₃) (assignment of the carbon signals was made based on the DEPT experiment). Anal. Calc. for C₂₂H₂₆I₂O₂S₂: C, 41.26; H, 4.09. Found: C, 41.44; H, 4.06.

### Example 22

### Preparation of 2,6-Dibromo-cyclopenta[2,1-b;3,4-b′]dithiophen-4-one

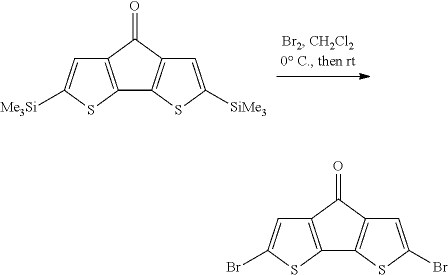 [SMILES: C[Si](C)(C)c1cc2c(s1)-c1sc([Si](C)(C)C)cc1C2=O ; O=C1c2cc(Br)sc2-c2sc(Br)cc21]

2,6-Bis-trimethylsilyl-cyclopenta[2,1-b;3,4-b′]dithiophen-4-one (3.0 mmol, 1.01 g) was dissolved in 20 mL of dichloromethane, cooled in ice-water bath and a solution of bromine (2.1 eq., 6.3 mmol, 1.01 g) in 10 mL of dichloromethane was added to a dark red solution. The reaction mixture became purple in color and after stirring for about 0.5 h it was allowed to warm to room temperature. Aqueous solution of Na₂S₂O₃ was added and organic solvent was removed by rotary evaporation. The dark purple solid was filtered off, washed with ethanol and dried. Crude product was obtained in 91.5% yield (0.96 g). This material was purified by column chromatography (150 mL of silica gel, CH₂Cl₂ as eluant; material was dissolved in boiling chloroform to apply to the column). Fractions with pure material were combined, the solvent was removed and product 2,6-dibromo-cyclopenta[2,1-b;3,4-b′]dithiophen-4-one was obtained as dark purple solid.

¹H NMR (CDCl₃, 400 MHz): δ 7.00 (s, 2H); ¹³C{¹H} NMR (CDCl₃, 100 MHz): δ 180.5 (quaternary C(O)), 148.7 (quaternary C), 139.5 (quaternary C), 124.4 (CH), 113.97 (quaternary C—Br) (assignment of the quaternary C and CH signals was made based on the DEPT experiment). Anal. Calc. for C₉H₂Br₂OS₂: C, 30.88; H, 0.58. Found: C, 30.87; H, 0.47.

Cyclic voltammograms of 2,6-dibromo-cyclopenta[2,1-b;3,4-b′]dithiophen-4-one in 0.1 M ⁿBu₄NPF₆ in THF, using Cp₂Fe internal standard at 0 V, 50 mV·s⁻¹ rate) gave a reversible reduction at E_{1/2}^{0/1-}=−1.52 V. In 0.1 M ⁿBu₄NPF₆ in CH₂Cl₂, using Cp₂Fe internal standard at 0 V, 50 mV·s⁻¹ rate) a semi-reversible oxidation was observed at E_{1/2}^{0/1+}=+1.05 V, and a reversible reduction E_{1/2}^{0/1-}=−1.48 V was also observed.

### Example 23

### Improved Procedure for the Preparation of 2,7-Bis-trimethylsilyl-benzo[2,1-b:3,4-b′]dithiophene-4,5-dione

The yield and purification procedure for the preparation of 2,7-bis-trimethylsilyl-benzo[2,1-b:3,4-b′]dithiophene-4,5-dione were improved by using slight excess of diethyl oxalate (1.3 eq.). This modification allows simplifying the isolation of the product from the crude mixture by recrystallization and also improves the yields up to 76-80% yields.

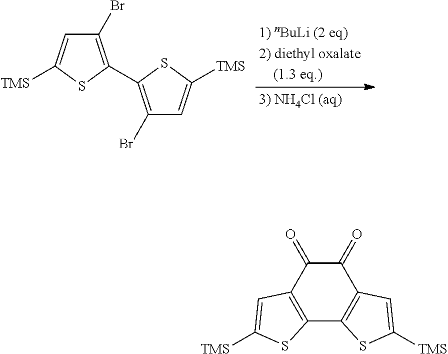 [SMILES: C[Si](C)(C)c1cc(Br)c(-c2sc([Si](C)(C)C)cc2Br)s1 ; C[Si](C)(C)c1cc2c(s1)-c1sc([Si](C)(C)C)cc1C(=O)C2=O]

3,3′-Dibromo-5,5′-bis-trimethylsilyl-2,2′-dithiophene (1a) (60.0 mmol, 28.11 g) was dissolved in anhydrous THF (240 mL), the solution was cooled in acetone/dry ice bath and n-butyllithium (2.87 M in hexanes, 2 eq., 120.0 mmol, 41.8 mL (caution! added in several portions with volume less than 20 mL) was added dropwise. The yellow-orange solution was stirred for 0.5 h and then transferred via cannula into a solution of diethyl oxalate (1.3 eq., 78.0 mmol, 11.40 g) in 200 mL of anhydrous THF (cooled in acetone/dry ice bath). After completion of the addition of the di-lithiated species to the diethyl oxalate, the orange-reddish mixture was stirred for 45 minutes and transferred via cannula into a solution of aqueous NH₄Cl. The dark red organic phase was separated, the aqueous phase was extracted with hexanes, and the combined organic phases were dried over MgSO₄. The solvents were removed by rotary evaporation and the crude product was heated to reflux with ˜500 ml of ethanol, cooled to room temperature, and dark-red needles were separated by the vacuum filtration (16.3 g, 76.7% yield). The mother liquor was subjected to rotary evaporation and the residue was recrystallized from ethanol to give additional amount of product (0.7 g, total yield 17.0 g, 79.9%).

¹H NMR (CDCl₃, 400 MHz): δ 7.60 (s, 2H), 0.36 (s, 18H, 6CH₃); ¹³C{¹H}NMR (CDCl₃, 100 MHz): δ 175.2 (quaternary C), 148.3 (quaternary C), 142.5 (quaternary C), 135.8 (quaternary C), 134.4 (CH), −0.44 (CH₃). HRMS (EI) calculated for C₁₆H₂₀O₂S₂Si₂ 364.0443; found 364.0469. Anal. Calc. for C₁₆H₂OO₂S₂Si₂: C, 52.70; H, 5.53. Found: C, 52.70; H, 5.36.

### Example 24

### 2,7-Dichloro-benzo[2,1-b:3,4-b′]dithiophene-4,5-dione

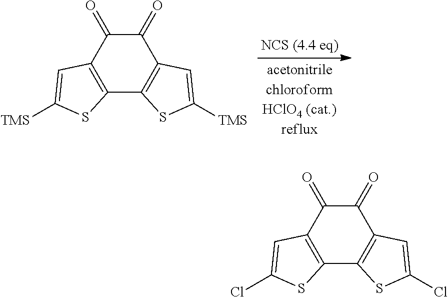 [SMILES: C[Si](C)(C)c1cc2c(s1)-c1sc([Si](C)(C)C)cc1C(=O)C2=O ; O=C1C(=O)c2cc(Cl)sc2-c2sc(Cl)cc21]

2,7-Bis-trimethylsilyl-benzo[2,1-b:3,4-b′]dithiophene-4,5-dione (4.0 mmol, 1.42 g) was mixed with N-chlorosuccinimide (2.2 eq., 8.8 mmol, 1.18 g) and 50 mL of acetonitrile was added. Dark red mixture was heated to reflux overnight and analyzed by TLC. Only starting material was detected and HClO₄ (0.05 mL, 69-72%) was added followed by addition of N-chlorosuccinimide (2.2 eq., 8.8 mmol, 1.18 g) and 10 mL of CHCl₃. Two new more polar red spots were detected by TLC (possible products of protiodesilylation), and the resulting mixture was refluxed overnight. Reaction mixture was cooled to room temperature, treated with aqueous solution of Na₂S₂O₃ and organic solvents were removed by rotary evaporation. Organic matter was extracted with dichloromethane, purple organic phases were dried over MgSO₄, and the solvent was removed by rotary evaporation. Almost black microcrystalline compound was obtained, 1.24 g, 107% crude yield (possible crystallization with the solvent).

This crude product was purified by column chromatography (200 mL of silica gel, chloroform as eluant). Fractions containing pure product were combined, the solvent was removed by rotary evaporation and very dark crystalline compound was obtained (0.59 g, 45.6% yield). This material was dissolved in toluene (˜40 mL) under reflux (purple solution) and allowed to cool to room temperature. Long very dark purple needles were obtained by vacuum filtration (2,7-Dichloro-benzo[2,1-b:3,4-b′]dithiophene-4,5-dione, 0.39 g, 66.1% recovery). First two fractions containing the product with minor impurities were combined separately, the solvent was removed and the residue was dissolved in boiling 2-propanol with addition of dichloromethane and purple solution was allowed to cool to room temperature. Long needles/blades were separated by vacuum filtration (0.063 g). Filtrates from both recrystallizations were combined separately, the solvents were removed and the residue was dissolved in boiling toluene and left to cool down for crystal growth.

.¹H NMR (CDCl₃, 400 MHz): δ 7.31 (s, 2H); ¹³C{¹H} NMR (CDCl₃, 100 MHz): δ 172.6 (quaternary C(O)), 141.0 (quaternary C), 134.5 (quaternary C), 132.2 (quaternary C), 126.2 (CH). HRMS (EI) calculated for C₁₀H₂Cl₂O₂S₂ 287.8873. found 287.8876. Anal. Calc. for C₁₀H₂Cl₂O₂S₂: C, 41.54; H, 0.70. Found: C, 41.54; H, 0.67.

A cyclic voltammogram (0.1 M ⁿBu₄NPF₆ in THF, Cp₂Fe internal standard at 0 V, 50 mV·s⁻¹ rate) of 2,7-dichloro-benzo[2,1-b:3,4-b′]dithiophene-4,5-dione was recorded: E_{1/2}^{0/1-}=−0.88 V (reversible), E_{1/2}^{1-/2-}−−1.68 V (reversible).

### Example 25

### 2,7-Di-bromo-benzo[2,1-b:3,4-b′]dithiophene-4,5-di-(1,3-dioxolane)

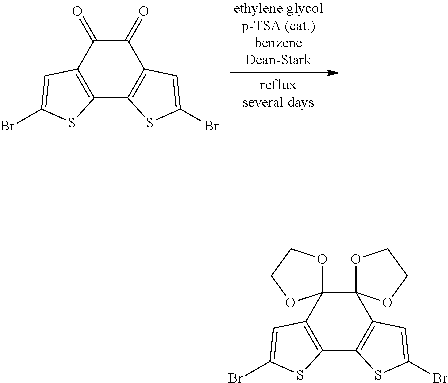 [SMILES: O=C1C(=O)c2cc(Br)sc2-c2sc(Br)cc21 ; Brc1cc2c(s1)-c1sc(Br)cc1C1(OCCO1)C21OCCO1]

2,7-Dibromo-benzo[2,1-b:3,4-b′]dithiophene-4,5-dione (18.0 mmol, 6.81 g), ethylene glycol (20 mL) and 100 mL of benzene were mixed together in a round bottom flask equipped with magnetic stir bar, Dean-Stark trap and a condenser. Catalytic amount (a few crystals) of p-TSA was added and the mixture was heated to reflux. Additional amount of ethylene glycol (40 mL) was added after a few hours and mixture was refluxed for 4 days until complete consumption of the starting material. Reaction mixture with greenish precipitate was cooled to room temperature, subjected to rotary evaporation (not a lot was removed), treated with water and greenish solid was separated by vacuum filtration (6.50 g, 77.5% crude yield). Organic matter in the filtrate was extracted with dichloromethane, combined with the greenish solid and purified by column chromatography (150 mL of silica gel, CH₂Cl₂:hexanes (2:1) as eluant). First fractions with slightly contaminated product were combined, the solvents were removed, the residue was heated with ˜250 mL of 2-propanol, cooled to room temperature and vacuum filtered (4.40 g, barely yellowish solid). Later fractions were kept separately, the solvents were removed by rotary evaporation and the residue was heated with ˜150 mL of 2-propanol to give off-white solid (2,7-Di-bromo-benzo[2,1-b:3,4-b′]dithiophene-4,5-di-(1,3-dioxolane), 1.21 g, combined yield 5.61 g, 85% recovery).

¹H NMR (CDCl₃, 400 MHz): δ 7.15 (s, 2H), 4.16 (m, 4H), 3.70 (m, 4H); ¹³C{¹H} NMR (400 MHz, CDCl₃): δ 135.9 (quaternary C), 133.5 (quaternary C), 128.1 (CH), 111.5 (quaternary C), 92.8 (quaternary C), 61.6 (CH₂). Anal. calc. for C₁₄H₁₀Br₂O₄S₂ C, 36.07; H, 2.16. Found: C, 36.35; H, 2.02.

### Example 26

### 2,7-Difluoro-benzo[2,1-b:3,4-b′]dithiophene-4,5-dione

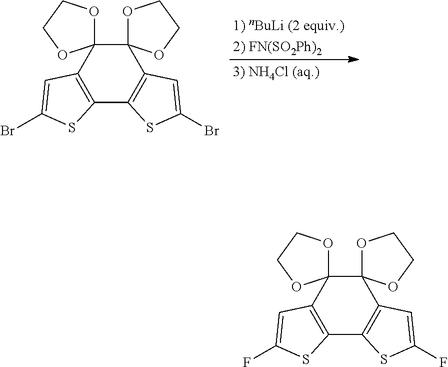 [SMILES: Brc1cc2c(s1)-c1sc(Br)cc1C1(OCCO1)C21OCCO1 ; Fc1cc2c(s1)-c1sc(F)cc1C1(OCCO1)C21OCCO1]

2,7-Di-bromo-benzo[2,1-b:3,4-b′]dithiophene-4,5-di-(1,3-dioxolane) (2.5 mmol, 1.165 g) was dissolved in 75 mL of anhydrous THF (nitrogen atmosphere) and the resulting yellowish solution was cooled in acetone/dry ice bath. n-Butyllithium (2.87 M in hexanes, 5.0 mmol, 1.75 mL) was added dropwise and yellowish solution became almost colorless suspension, which became light pink after stirring for a few minutes. The reaction mixture was stirred for 15 minutes and a solution of N-fluorobenzenesulfonimide (2.1 eq., 5.25 mmol, 1.66 g) in 25 mL of anhydrous THF was added dropwise. Reaction mixture became orange solution. After stirring for 10 minutes additional amount of N-fluorobenzenesulfonimide (0.16 g) was added, the reaction mixture was allowed to warm to room temperature and then treated with water. Organic phase was separated, the aqueous phase was extracted with dichloromethane and combined organic phases (yellow-brownish) were subjected to rotary evaporation. The residue was mixed with chloroform, heated to reflux and insoluble matter was separated by vacuum filtration. Filtrate was column chromatographed (˜250 mL of silica gel, dichloromethane as eluant). Fractions containing the product were combined, the solvent was removed by rotary evaporation and beige solid was obtained (microcrystalline compound, 0.46 g, 53.5% yield). The compound on the sides of the flask was mixed with 2-propanol, heated to reflux to dissolve the solid and cooled. Little colorless crystals formed on cooling indicating that 2-propanol could be a good solvent for recrystallization. Part of the solid (0.21 g) was recrystallized from 2-propanol, and purified product was obtained as yellowish large crystals (0.16 g, 76.2% recover).

¹H NMR (CDCl₃, 400 MHz): δ 6.61 (s, 2H), 4.13 (m, 4H), 3.71 (m, 4H); ¹³C{¹H} NMR (CDCl₃, 100 MHz): δ 165.2 (d, J=295 Hz, quaternary C—F), 131.7 (quaternary C), 120.8 (quaternary C), 105.8 (d, J=11 Hz, CH), 92.7 (quaternary C), 61.6 (CH₂). ¹⁹F NMR (CDCl₃, 376.3 MHz): δ −129.9 (1,1,2-trichlorotrifluoroethane was used as a reference with δ at −71.75 ppm (t)). HRMS (EI) calculated C₁₄H₁₀F₂O₄S₂ 343.9989. found 343.9982. Anal. Calc. for C₁₄H₁₀F₂O₄S₂: C, 48.83; H, 2.93. Found: C, 48.73; H, 2.90.

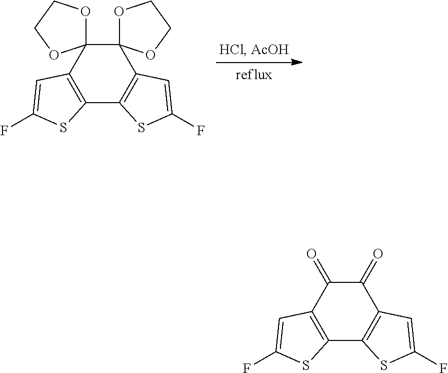 [SMILES: Fc1cc2c(s1)-c1sc(F)cc1C1(OCCO1)C21OCCO1 ; O=C1C(=O)c2cc(F)sc2-c2sc(F)cc21]

2,7-Difluoro-benzo[2,1-b:3,4-b′]dithiophene-4,5-di-(1,3-dioxolane) (0.5 mmol, 0.172 g) was mixed with acetic acid (10 mL) and the resulting mixture was heated to reflux. HCl (1 mL) was added dropwise, and yellowish mixture became purple within a few minutes. The mixture was refluxed for ˜10 minutes, analyzed by TLC (CHCl₃ as eluant) and complete consumption of the starting material was confirmed (a new purple spot of the product was detected as well). The reaction mixture was cooled to room temperature, treated with water and dark precipitated was separated by vacuum filtration, washed with water, then ethanol and dried (, 0.144 g, 113% crude yield, probably still contained some solvents). This material was recrystallized from toluene-hexanes and very dark purple needles were obtained (0.123 g, 96% yield). Some needles looked reasonable for single crystal X-ray analysis and were separated from the main batch.

2,7-Difluoro-benzo[2,1-b:3,4-b′]dithiophene-4,5-dione: ¹H NMR (CDCl₃, 400 MHz): δ 6.89 (s, 2H); ¹³C{¹H} NMR (CDCl₃, 100 MHz): δ 172.9 (quaternary C(O)), 165.4 (d, J=300 Hz, quaternary C—F), 133.0 (quaternary C), 132.2 (quaternary C), 107.3 (d, J=11 Hz, CH) (assignment of the CH and quaternary carbons was made based on DEPT-135 analysis). HRMS (EI) calculated for C₁₀H₂F₂O₂S₂ 255.9464. found 255.9476. Anal. Calc. for C₁₀H₂F₂O₂S₂: C, 46.87; H, 0.79. Found: C, 47.36; H, 0.83.

### Example 27

### 2,7-Bis-trimethylsilylethynyl-benzo[2,1-b:3,4-b′]dithiophene-4,5-dione

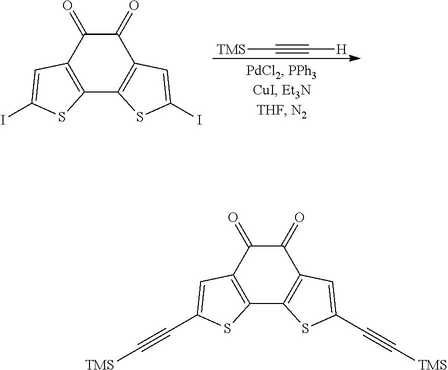 [SMILES: O=C1C(=O)c2cc(I)sc2-c2sc(I)cc21 ; C#C[Si](C)(C)C ; C[Si](C)(C)C#Cc1cc2c(s1)-c1sc(C#C[Si](C)(C)C)cc1C(=O)C2=O]

2,7-Diiodo-benzo[2,1-b:3,4-b′]dithiophene-4,5-dione (1.0 mmol, 0.472 g), PdCl₂ (0.04 eq., 0.04 mmol, 0.007 g), PPh₃ (0.1 eq., 0.1 mmol, 0.026 g) and Et₃N (2.2 eq., 2.2 mmol, 0.22 g) were mixed in an oven-dried Schlenk flask under nitrogen atmosphere. Anhydrous THF (30 mL) was added followed by addition of trimethylsilylacetylene (2.2 eq., 2.2 mmol, 0.22 g) and CuI (0.012 eq., 0.012 mmol, 2.3 mg). The mixture was heated (58° C. bath temperature initially, then 40-45° C.), but no reaction was observed by TLC analysis (CH₂Cl₂ as eluant) after ˜1.5 h of heating. Additional amount of Et₃N (0.3 mL) was added, followed by addition of trimethylsilylacetylene (2.4 mmol, 0.24 g). After stirring at heating (47-49° C. bath temperature) for 4 hours no reaction was observed based on TLC analysis and additional amount of CuI (6.5 mg) was added. After ˜20 minutes of stirring dark red-purple mixture became yellowish-greenish and the mixture was left to stir overnight (40° C., nitrogen atmosphere). The yellow-greenish mixture was cooled to room temperature and treated with water. Organic phase became dark purple-brown, brine was added and organic phase was separated. The aqueous phase was extracted with diethyl ether several times and combined organic phases were dried over MgSO₄. The drying agent was filtered off, the solvents were removed by rotary evaporation and the residue was purified by column chromatography (150 mL of silica gel, CH₂Cl₂ as eluant). Material came out contaminated, and the fractions with the product were combined, subjected to rotary evaporation and the residue was recrystallized from 2-propanol. Product was obtained as very dark needles (0.058 g, 14%). The column was eluted with CHCl₃:EtOAc and purple solution was collected, subjected to rotary evaporation and purified by column chromatography (150 mL of silica gel, CHCl₃ as eluant). Combined fractions were subjected to rotary evaporation, and the residue was recrystallized from ˜15 mL of EtOH. Very dark crystals were separated by vacuum filtration and additional amount of product was obtained (0.029 g, 7.0%).

¹H NMR (CDCl₃, 400 MHz): δ 7.53 (s, 2H), 0.27 (s, 18H); ¹³C{¹H} NMR (CDCl₃, 100 MHz): δ 173.6, 142.9, 135.0, 132.1 (CH), 124.5, 104.5, 95.0. HRMS (EI) calculated for C₂₀H₂₀O₂S₂Si₂ 412.0443. found 412.0449. Anal. Calc. for C₂₀H₂₀O₂S₂Si₂: C, 58.21; H, 4.88. Found: C, 57.36; H, 4.87 (ΔC −0.85)

Cyclic voltammograms of 2,7-bis-trimethylsilanylethynyl-benzo[2,1-b:3,4-b′]dithiophene-4,5-dione: were recorded (0.1 M ⁿBu₄NPF₆ in THF, Cp₂Fe internal standard at 0 V, 50 mV·s⁻¹ rate) and showed E_{1/2}^{0/1-}=−0.91V (reversible), and E_{1/2}^{1-/2-}=−1.60 V (reversible).

### Example 28

### 2,7-ethynyl-benzo[2,1-b:3,4-b′]dithiophene-4,5-dione

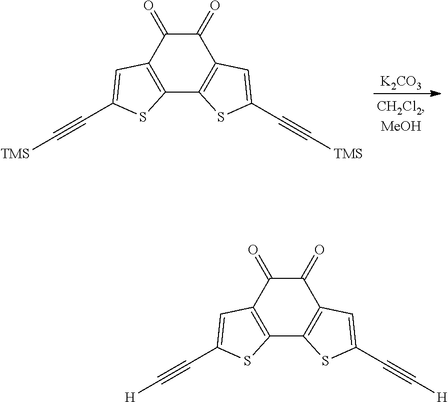 [SMILES: C[Si](C)(C)C#Cc1cc2c(s1)-c1sc(C#C[Si](C)(C)C)cc1C(=O)C2=O ; "K2CO3" ; C#Cc1cc2c(s1)-c1sc(C#C)cc1C(=O)C2=O]

2,7-Bis-trimethylsilylethynyl-benzo[2,1-b:3,4-b′]dithiophene-4,5-dione (0.07 mmol, 0.029 g) was dissolved in a mixture of dichloromethane-methanol (10:10 mL) and K₂CO₃ (3.0 eq., 0.12 mmol, 0.029 g) was added to a dark blue-purple solution at room temperature. The reaction mixture was stirred for about 1 h and treated with water. Organic phase was removed, aqueous phase was extracted with dichloromethane and combined organic phases were dried over MgSO₄. The drying agent was filtered off, the solvent was removed and the crude product was purified by column chromatography (50 mL of silica gel, CH₂Cl₂ as eluant). Solvent was removed from combined fractions and product was obtained as dark microcrystalline solid (100% yield, 0.019 g).

¹H NMR (CDCl₃, 400 MHz): δ 7.61 (s, 2H), 3.56 (s, 2H); ¹³C{¹H} NMR (CDCl₃, 100 MHz): δ 173.50 (quaternary C(O)), 143.03 (quaternary C), 132.72 (CH), 123.41 (quaternary C), 85.69 (CH), 74.70 (quaternary C) (assignment of the quaternary and CH signals was made based on the DEPT experiment).

Cyclic voltammograms of 2,7-ethynyl-benzo[2,1-b:3,4-b′]dithiophene-4,5-dione (0.1 M ⁿBu₄NPF₆ in THF, Cp₂Fe internal standard at 0 V, 50 mV·s⁻¹ rate) showed two reversible reductions, E_{1/2}^{0/1-}=−0.91 V, E_{1/2}^{1-/2-}=−1.60 V.

### Example 29

### 2,7-ethynyl-benzo[2,1-b:3,4-b′]dithiophene-4,5-dione

### Step 1-4,4′-dibromo-2,2′-bis(4-hexyl-5-(trimethylsilyl)thiophen-2-yl)-5,5′-bithiazole

Lithium diisopropylamide (LDA) (2.2 eq., 0.37 M, 6 ml) was prepared from diisopropylamine (2.4 mmol, 0.24 g), n-butyllithium (2.5 M in hexanes, 2.2 mmol, 0.9 ml) and 5 ml of anhydrous THF. 2-(5-Trimethylsilyl-3-n-hexyl-thiophen-2-yl)-5-bromothiazole (1.0 mmol, 0.40 g, see Example 7) was dissolved in 20 ml of anhydrous THF and the yellowish solution was cooled in acetone/CO₂ bath (nitrogen atmosphere). Freshly prepared LDA (0.37 M in THF, 1.1 eq., 3 ml) was added dropwise to the bromothiazole derivative and the reaction mixture became light purple in color. The reaction mixture was stirred for 20 minutes and a small aliquot was treated with hexanes:MeOH, organic solvents were removed and the residue was analyzed by GC/MS analysis. The completion of the BCHD reaction was confirmed and CuCl₂ (1.1 eq., 0.148 g) was added in one portion to the purple reaction mixture. After stirring for 5 minutes the color changed to yellowish-green and the mixture was slowly warmed to room temperature without cooling bath removal.

Hexanes and water were added, the organic phase was removed and the aqueous phase was extracted with Et₂O (3×15-20 ml). The combined organic phases were dried over MgSO₄ and the solvents were removed by rotary evaporation to give crude product as dark yellow solid. This crude product was purified by column chromatography (50 ml of silica gel, hexanes:CH₂Cl₂ (3:2) and bright yellow-orange solid was obtained (0.27 g). Minor impurities were detected by the TLC analysis and material was further purified by the column chromatography (100 ml of silica gel, Hexanes:CH₂Cl₂ (35:15). The solvents were removed from combined fractions and product was obtained as yellow-orange oil which solidified on standing.

4,4′-Dibromo-2,2′-bis(4-hexyl-5-trimethylsilyl-thiophen-2-yl)-5,5′-bithiazole; ¹H NMR (CDCl₃, 400 MHz): δ 7.53 (s, 2H), 2.66 (t, J=8.0 Hz, 4H), 1.62 (m, 4H), 1.45-1.30 (m, 12H) 0.98 (t, J=6.9 Hz, 6H), 0.38 (s, 18H); ¹³C{¹H} NMR (CDCl₃, 100 MHz): δ 162.1 (quaternary C), 151.4 (quaternary C), 139.0 (quaternary C), 138.5 (quaternary C), 130.5 (CH), 127.6 (quaternary C), 121.0 (quaternary C), 31.7 (CH₂), 31.6 (CH₂), 31.3 (CH₂), 29.3 (CH₂), 22.6 (CH₂), 14.1 (CH₃), 0.14 (CH₃) (assignment of the quaternary, CH, CH₂ and CH₃ signals was made based on the DEPT experiment). HRMS (EI) calculated for C₃₂H₄₆Br₂N₂S₄Si₂ 800.0449. found 800.0420. Anal. Calc. for C₃₂H₄₆Br₂N₂S₄Si₂: C, 47.87; H, 5.77; N, 3.49. Found: 47.72; H, 5.77; N, 3.47.

### Step 2

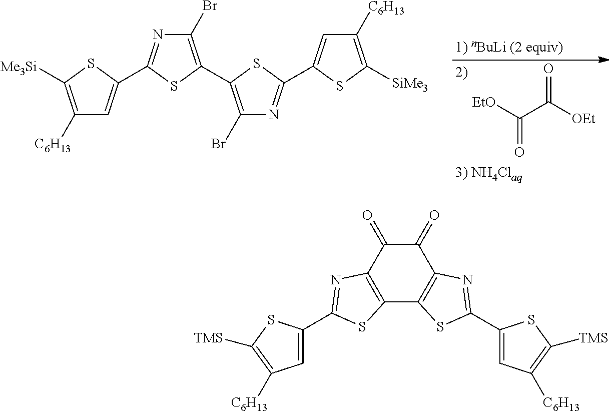

4,4′-Dibromo-2,2′-bis(4-n-hexyl-5-trimethylsilyl-thiophen-2-yl)-5,5′-bithiazole (0.5 mmol, 0.401 g) was dissolved in 30 mL of anhydrous THF under nitrogen atmosphere and the resulting bright yellow solution was cooled in acetone/dry ice bath. n-Butyllithium (2.85 M in hexanes, 1.0 mmol, 0.35 mL) was added dropwise, and reaction mixture became orange-red. After stirring for 0.5 h this solution was transferred via cannula into a solution of diethyl oxalate (1.2 eq., 0.6 mmol, 0.09 g) in 50 mL of anhydrous THF cooled in acetone/dry ice bath. Very dark red-orange solution became yellow-red-brownish. After stirring for 1 h only trace amount of the desired product was detected by TLC analysis and the mixture was allowed to warm to 0° C. After stirring for 3 hours additional amount of diethyl oxalate (0.2 mL) was added and the mixture was left to stir overnight. The reaction mixture was treated with aqueous NH₄Cl, dark brown organic phase was separated and the aqueous phase was extracted with dichloromethane. Combined organic phases were dried over MgSO₄, the organic solvents were removed by rotary evaporation and the residue was purified by column chromatography (100 mL of silica gel, CH₂Cl₂:EtOAc (30:1, 20:1, 10:1). All blue or green fractions were combined, the solvents were removed and the product (still impure) was obtained as green-blue film (˜50 mg). This material was further purified by column chromatography (˜50 mL of silica gel, CH₂Cl₂ as eluant). Fractions with material (blue in color) were combined, the solvent was removed by rotary evaporation and the product was obtained as blue-green film (˜30 mg, <10% yield).

¹H NMR (CDCl₃, 400 MHz): δ 7.59 (s, 2H), 2.65 (t, J=8.0 Hz, 4H), 1.61 (m, 4H), 1.42-1.30 (m, 12H), 0.93, (t, J=6.6 Hz, 6H), 0.38 (s, 18H); ¹³C{¹H}NMR (CDCl₃, 100 MHz): 172.5, 162.0, 151.6, 147.9, 140.9, 137.8, 136.8, 132.0 (CH), 31.7 (CH2), 31.6 (CH2), 31.3 (CH2), 29.3 (CH2), 22.6 (CH2), 14.1 (CH3), 0.1 (CH3). HRMS (EI) calculated for C34H46N2O2S4Si2 698.1981. Found: 698.1970. (M+2 ion was also observed as a major ion: calculated for C₃₄H₄₈N₂O₂Si₂S₄ 700.2137. found 700.2090). Anal. Calc. for C₃₄H₄₆N₂O₂S₄Si₂: C, 58.41; H, 6.63; N, 4.01. Found: 58.50; H, 6.64,N, 4.11.

### Example 30

### Synthesis of 2,7-bis(perfluorobenzoyl)benzo[1,2-b:3,4-b′]dithiophene-4,5-dione

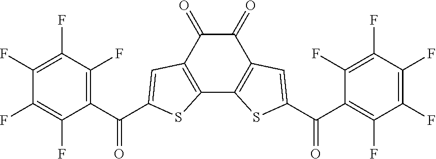 [SMILES: O=C1C(=O)c2cc(C(=O)c3c(F)c(F)c(F)c(F)c3F)sc2-c2sc(C(=O)c3c(F)c(F)c(F)c(F)c3F)cc21]

### Step 1

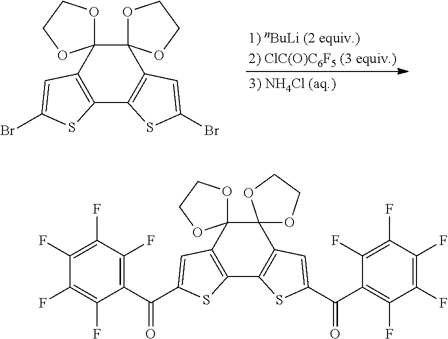 [SMILES: Brc1cc2c(s1)-c1sc(Br)cc1C1(OCCO1)C21OCCO1 ; O=C(c1cc2c(s1)-c1sc(C(=O)c3c(F)c(F)c(F)c(F)c3F)cc1C1(OCCO1)C21OCCO1)c1c(F)c(F)c(F)c(F)c1F]

2,7-Dibromo-benzo[2,1-b:3,4-b′]dithiophene-4,5-di-(1,3-dioxolane) (3.0 mmol, 1.40 g) was dissolved in 75 mL of anhydrous THF, and solution was cooled in acetone/dry ice bath. n-Butyllithium (2.85 M in hexanes, 6.0 mmol, 2.11 mL) was added dropwise and purple suspension formed. The reaction mixture was stirred for ˜40 minutes and transferred via cannula into a solution of pentafluorobenzoyl chloride (9.0 mmol, 2.07 g) in 75 mL of anhydrous THF cooled in acetone/dry ice bath. Yellow-brown solution formed. After stirring for ˜2 h the cooling bath was removed, the mixture was treated with aqueous solution of NH₄Cl, and organic phase was removed. Aqueous phase was extracted with CH₂Cl₂ and combined organic phases were dried over MgSO₄. The drying agent was filtered off, and the solvents were removed by rotary evaporation. The crude product was purified by column chromatography (250 mL of silica gel, hexanes:CH₂Cl₂ as eluant. Fractions with pure product were combined, and the solvents were removed from yellow solution, and yellow solid was obtained. This material contained some solvent, and 100 mg was recrystallized from 2-propanol (˜75 mL). Yellow solid (83 mg) was obtained. Anal. Calc. for C₂₈H₁₀F₁₀O₆S₂: C, 48.28; H, 1.45. Found: C, 48.14; H, 1.54.

¹H NMR (CDCl₃, 400 MHz): δ 7.57 (s, 2H), 4.17 (m, 4H), 3.70 (m, 4H); ¹³C{¹H} NMR (CDCl₃, 100 MHz): δ 176.0, 142.9, 141.7, 140.1, 133.6 (CH), 61.5 (CH₂) (multiplets for C—F carbons were observed as weak signals at 145.1, 142.6, 139.0, 136.5). ¹⁹F NMR (CDCl₃, 376.3 MHz): δ −139.4 (m, 4F), −148.9 (appears as poorly resolved tt, 2F), −159.0 (appears as not well resolved qt, 4F) (1,1,2-trichlorotrifluoroethane was used as a reference with δ at −71.75 ppm (t)). HRMS (EI) calculated for C₂₈H₁₀F₁₀O₆S₂ 695.9759; found 695.9733.

Step 2:

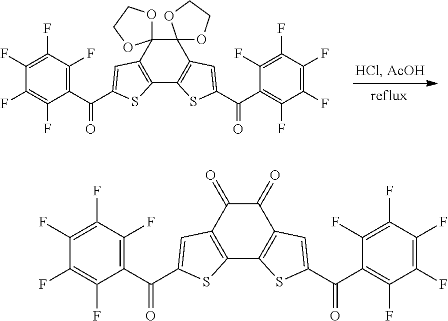 [SMILES: O=C(c1cc2c(s1)-c1sc(C(=O)c3c(F)c(F)c(F)c(F)c3F)cc1C1(OCCO1)C21OCCO1)c1c(F)c(F)c(F)c(F)c1F ; O=C1C(=O)c2cc(C(=O)c3c(F)c(F)c(F)c(F)c3F)sc2-c2sc(C(=O)c3c(F)c(F)c(F)c(F)c3F)cc21]

2,7-Bis-pentafluorobenzoyl-benzo[2,1-b:3,4-b′]dithiazole-4,5-di-(1,3-dioxolane) (0.4 mmol, 0.279 g) was mixed with 50 mL of acetic acid and the mixture was heated to reflux. HCl (˜5 mL) was added to a yellow solution and the reaction mixture became orange and then red-orange. After reflux for 1 h the mixture was cooled to room temperature and only small amount of precipitate formed. The mixture was heated to reflux and water was added until precipitation was observed. The reaction mixture was cooled, and orange solid was separated, washed with water, ethanol and dried, (0.190 g, 78.2%). This material was purified for mobility measurement by column chromatography (100 mL of silica gel, CH₂Cl₂ as eluant). Middle fractions with the product were combined, the solvent was removed and orange-red powder was obtained (0.109 g, 77.9% recovery).

Bis(perfluorobenzoyl)benzo[1,2-b:3,4-b′]dithiophene-4,5-dione. ¹H NMR (CDCl₃, 400 MHz): δ 7.88 (s, 2H); ¹³C{¹H} NMR (CDCl₃, 100 MHz): δ 176.5 (quaternary C(O)), 173.1 (quaternary C(O)), 148.7, 144.3, 137.3, 134.4 (weak C—F carbons were detected as multiplets at 145.2, 142.7, 139.2, 136.6). ¹⁹F NMR (CDCl₃, 376.3 MHz): δ −139.1 (m, 4F), −147.1 (tt, J=20.7 Hz, 3.4 Hz, 2F), −158.2 (m, 4F) (1,1,2-trichlorotrifluoroethane was used as a reference with δ at −71.75 ppm (t)). HRMS (EI) calculated for C₂₄H₂F₁₀O₄S₂ 607.9235; found 607.9216 (M+2H at 609.9 was observed with ˜80% intensity with respect to molecular ion). Anal. Calc. for C₂₄H₂F₁₀O₄S₂: C, 47.38; H, 0.33. Found: C, 47.13; H, 0.34.

The above specification, examples and data provide exemplary description of the manufacture and use of the various compositions and devices of the inventions, and methods for their manufacture and use. In view of those disclosures, one of ordinary skill in the art will be able to envision many additional embodiments of the inventions disclosed and claimed herein to be obvious, and that they can be made without departing from the spirit and scope of the invention. The claims hereinafter appended define some of those embodiments.

1. A method for synthesizing a bishalo-bisheteroaryl compound comprising the structure 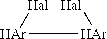
wherein HAr is an optionally substituted five or six membered heteroaryl ring comprising at least one ring carbon atom and at least one ring heteroatom, and Hal is a halogen: and wherein the steps of the method comprise:
a) providing an optionally substituted precursor compound comprising a halo-heteroaryl ring having an Hal substituent at a first position on the HAr ring;
b) treating the precursor compound with a strongly basic compound to induce the isomerization of the precursor compound to produce an intermediate compound wherein the Hal atom is bound to a different position on the HAr ring;
c) treating the intermediate compound with an oxidizing agent so as to form a carbon-carbon bond between two intermediate compounds and thereby form the bishalo-bisheteroaryl compound.

2. The method of claim 1 wherein Hal is Br or I.

3. The method of claim 1 wherein HAr is an optionally substituted five membered heteroaryl ring.

4. The method of claim 1 wherein HAr and Hal of the precursor compound comprise the structure 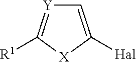
wherein
a) R¹ is a halide, or a C₁-C₃₀ organic radical selected from optionally substituted alkyl, alkynyl, aryl, and heteroaryl, or —Sn(R²)₃, —Si(R²)₃, —Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b) X is O, S, Se, or NR³ wherein R³ is a C₁-C₁₈ alkyl, perfluoroalkyl, aryl, or heteroaryl; and
c) Y is CH, CR⁴, or N, wherein R⁴ is a C₁-C₁₈ alkyl, aryl, or heteroaryl.

5. The method of claim 1 wherein HAr and Hal of the precursor compound comprise the structure 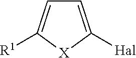
wherein
a) R¹ is a halide, or a C₁-C₃₀ organic radical selected from optionally substituted alkyl, alkynyl, aryl, and heteroaryl, or —Sn(R²)₃, —Si(R²)₃, —Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b) X is S, Se, or NR³ wherein R³ is a C₁-C₁₈ alkyl, perfluoroalkyl, aryl, or heteroaryl.

6. The method of claim 1 wherein HAr and Hal of the precursor compound comprise the structure 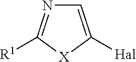
wherein
a) R¹ is a C₁-C₃₀ organic radical selected from optionally substituted alkyl, alkynyl, aryl, or heteroaryl, or —Sn(R²)₃, —Si(R²)₃, —Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms; and
b) X is S or NR³ wherein R³ is a C₁-C₁₈ alkyl, perfluoroalkyl, aryl, or heteroaryl.

7. The method of claim 1 wherein HAr and Hal of the precursor compound comprise the structure 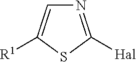
wherein
a) R¹ is a halide, or a C₁-C₃₀ organic radical selected from alkyl, alkynyl, aryl, heteroaryl, —Sn(R²)₃, —Si(R²)₃, —Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group to form a ring bridging the oxygen atoms.

8. The method of any one of claims 4-7 wherein R¹ is a C₁-C₃₀ aryl or heteroaryl optionally substituted by one to four ring substituents independently selected from halides, alkyl, alkynyl, perfluoroalkyl, alkoxide, perfluoroalkoxide, —Sn(R²)₃, —Si(R²)₃, —Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group to form a ring bridging the oxygen atoms.

9. The method of any one of claims 4-7 wherein R¹ is 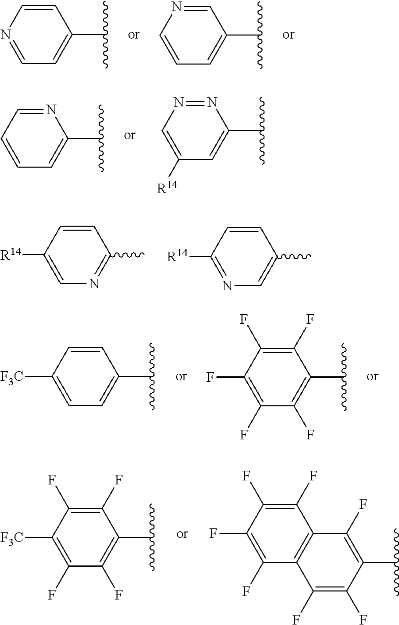
wherein R^{N} is hydrogen or a C₁-C₁₈ alkyl, perfluoroalkyl, or alkoxy group.

10. The method of any one of claims 4-7 wherein R¹ is 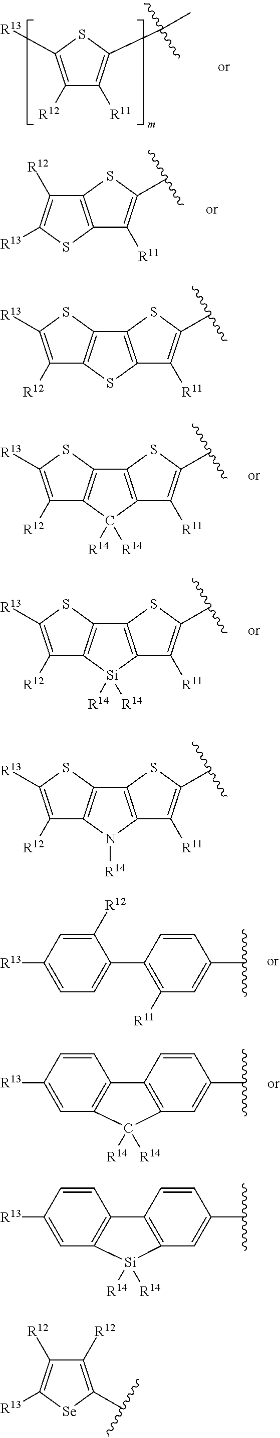 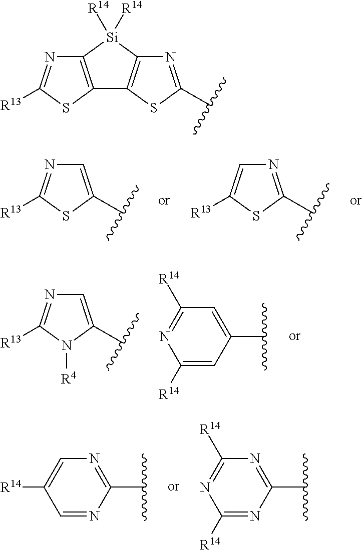
wherein m is 1, 2, 3, or 4, and R¹¹, R¹², R¹⁴ are a C₁-C₁₈ alkyl, perfluoroalkyl, alkoxy, or perfluoroalkoxy group, and R¹³ is hydrogen, —B(—OR²¹)₂, Si(R²)₃, Si(OR²)₃ or Sn(R²)₃, wherein each R² is an independently selected alkyl or aryl, and each R²¹ is an independently selected alkyl or aryl, or the R²¹ groups together form an optionally substituted alkylene group to form a ring bridging the oxygen atoms.

11. The method of any one of claims 1-10 wherein the strongly basic compound is an alkyl lithium compound.

12. The method of any one of claims 1-10 wherein the strongly basic compound is a lithium dialkylamide compound.

13. The method of any one of claims 1-10 wherein the oxidizing agent is a Cu(II) salt.

14. The method of any one of claims 1-10 wherein the bishalo-bisheteroaryl compound is a 2,2′-bishalo-1,1′-bisheteroaryl compound.

15. The method of claim 4 wherein the bishalo-bisheteroaryl compound has the structure 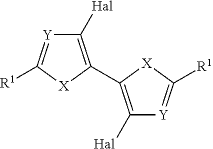
wherein
a) R¹ is a halide, or a C₁-C₃₀ organic radical selected from optionally substituted alkyl, alkynyl, aryl, and heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b) X is O, S, Se, or NR³ wherein R³ is a C₁-C₁₈ alkyl, perfluoroalkyl, aryl, or heteroaryl; and
c) Y is CH, CR⁴, or N, wherein R⁴ is a C₁-C₁₈ alkyl, aryl, or heteroaryl.

16. The method of claim 1-3 wherein the bishalo-bisheteroaryl compound has one of the structures 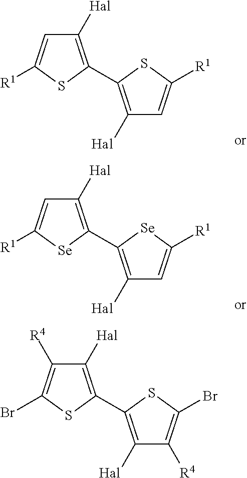
wherein
a) R¹ is hydrogen or a halide, or a C₁-C₃₀ organic radical selected from alkyl, alkynyl, aryl, heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b) R⁴ is a C₁-C₁₈ alkyl, aryl, or heteroaryl.

17. The method of any one of claims 1-2 wherein the bishalo-bisheteroaryl compound has one of the structures 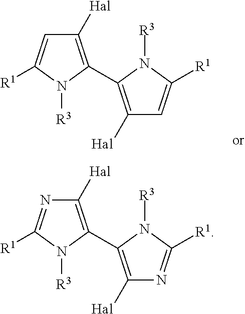
wherein
a) R¹ is hydrogen or a halide, or a C₁-C₃₀ organic radical selected from optionally substituted alkyl, alkynyl, aryl, heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b) R³ is a C₁-C₁₈ alkyl, perfluoroalkyl, aryl, or heteroaryl.

18. The method of any one of claims 1-3 wherein the bishalo-bisheteroaryl compound has one of the structures 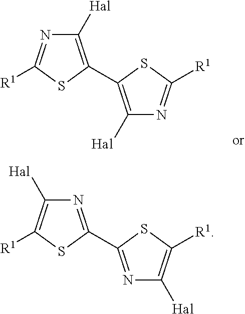
wherein R¹ is hydrogen or a halide, or a C₁-C₃₀ organic radical selected from alkyl, alkynyl, aryl, heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms.

19. The method of any one of claims 1-2 wherein the bishalo-bisheteroaryl compound has one of the structures

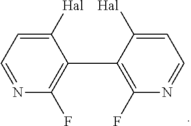

20. A method for synthesizing a fused tricyclic compound comprising the structure 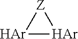
wherein
a) HAr is as defined in any one of claims 1-9,
b) Z is S, Se, NR⁵, C(O), C(O)C(O), Si(R⁵)₂, SO, SO₂, PR⁵, P(O)R⁵, BR⁵, or C(R⁵)₂ wherein R⁵ is a C₁-C₅₀ organic radical selected from optionally substituted alkyl, perfluoroalkyl, aryl, and heteroaryl, and wherein the method comprises the steps of any one of claims 1-17, and then further comprises the steps of
c) optionally treating the bishalo-bisheteroaryl compound with an organometallic compound to exchange a metal for the Hal substituents, and form a bismetallo-bisheteroaryl compound, and
d) reacting the bismetallo-bisheteroaryl compound with a suitable electrophile, or reacting the bishalo-bisheteroaryl compound or bismetallo-bisheteroaryl compound with a nucleophile, to introduce the Z group, or a precursor thereof suitable for forming the fused tricyclic compound.

21. The method of claim 20 wherein the organometallic compound is an alkyl lithium compound or lithium diorganoamide.

22. The method of claim 20 wherein the organometallic compound is a transition metal compound.

23. The method of claim 20 wherein the electrophile is a compound V—R⁶—V′, where R⁶ is selected from S, Se, NR⁵, C(O), C(O)C(O), Si (R⁵)₂, SO, SO₂, PR⁵, P(O)R⁵, BR⁵, or C(R⁵)₂, V and V′ are leaving groups or V and V′ together form a leaving group suitable for a condensation reaction with the bismetallo-bisheteroaryl compound to form the fused tricyclic compound.

24. The method of claim 20 wherein the fused tricyclic compound has the structure 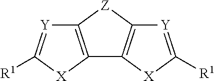
wherein
a) R¹ is hydrogen, a halide, or a C₁-C₃₀ organic radical selected from optionally substituted alkyl, alkynyl, aryl, and heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms;
b) X is O, S, Se, or NR³ wherein R³ is a C₁-C₁₈ alkyl, perfluoroalkyl, aryl, or heteroaryl; and
c) Y is CH, CR⁴, or N, wherein R⁴ is a C₁-C₁₈ alkyl, aryl, or heteroaryl; and
d) Z is S, Se, NR⁵, C(O), C(O)C(O), Si(R⁵)₂, SO, SO₂, PR⁵, P(O)R⁵, BR⁵, or C(R⁵)₂, wherein R⁵ is a C₁-C₅₀ organic radical selected from optionally substituted alkyl, perfluoroalkyl, aryl, and heteroaryl.

25. The method of claim 20 wherein the fused tricyclic compound has the structure 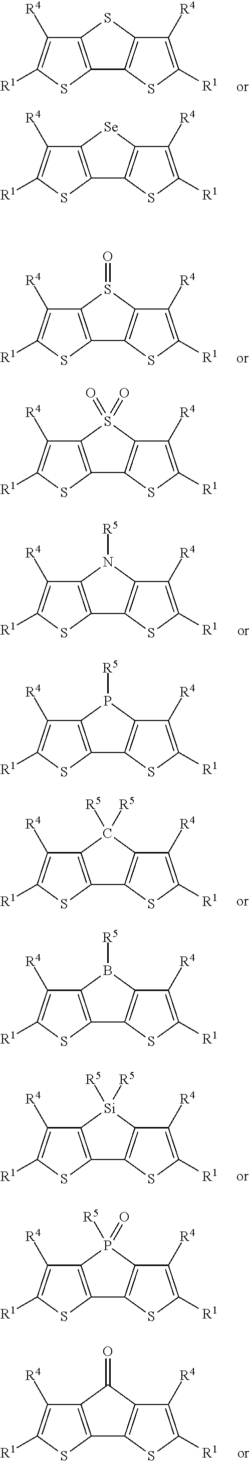 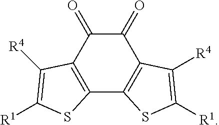
wherein R¹ is hydrogen or a halide, or a C₁-C₃₀ organic radical selected from optionally substituted alkyl, alkynyl, aryl, and heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms, R⁴ is hydrogen or optionally a C₁-C₁₈ alkyl group, and R⁵ is a C₁-C₅₀ organic radical selected from alkyl, aryl, heteroaryl.

26. The method of claim 25 wherein R¹ is 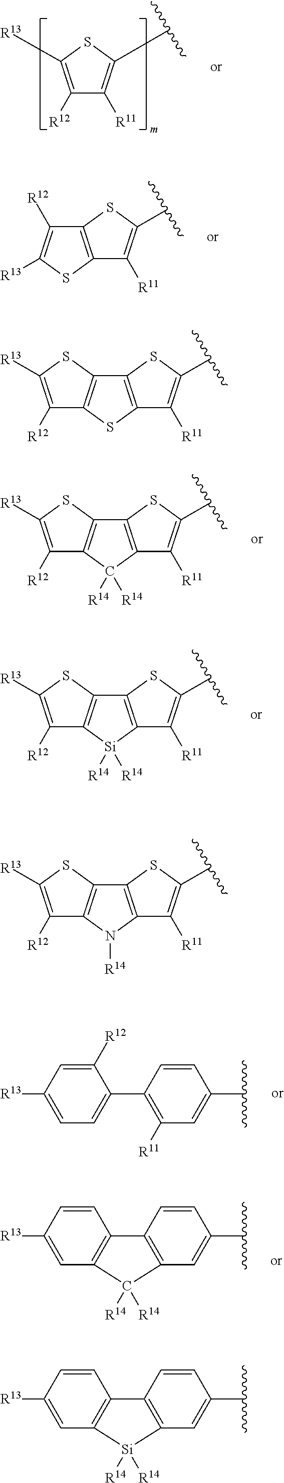 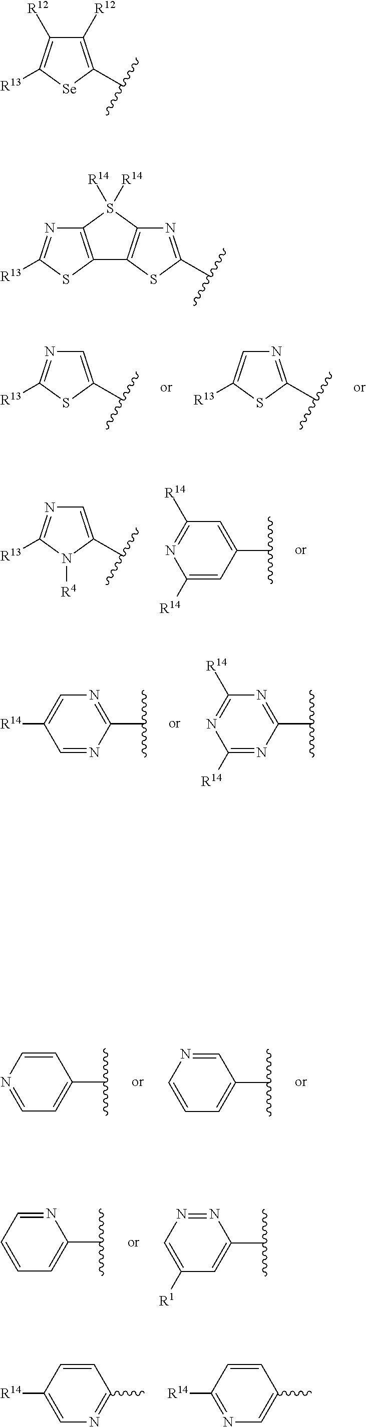 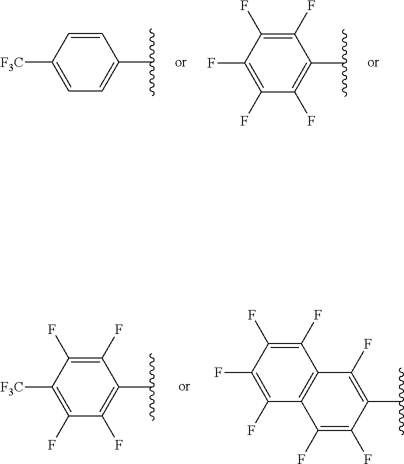
wherein m is 1, 2, 3, or 4, and R¹¹, R¹², R¹⁴ are a C₁-C₁₈ alkyl, perfluoroalkyl, alkoxy, or perfluoroalkoxy group, and R¹³ is hydrogen, —B(—OR²¹)₂, Si(R²)₃, Si(OR²)₃, or Sn(R²)₃, wherein each R² is an independently selected alkyl or aryl, and each R²¹ is an independently selected alkyl or aryl, or the R²¹ groups together form an optionally substituted alkylene group bridging the oxygen atoms.

27. The method of claim 20 wherein the fused tricyclic compound has the structure 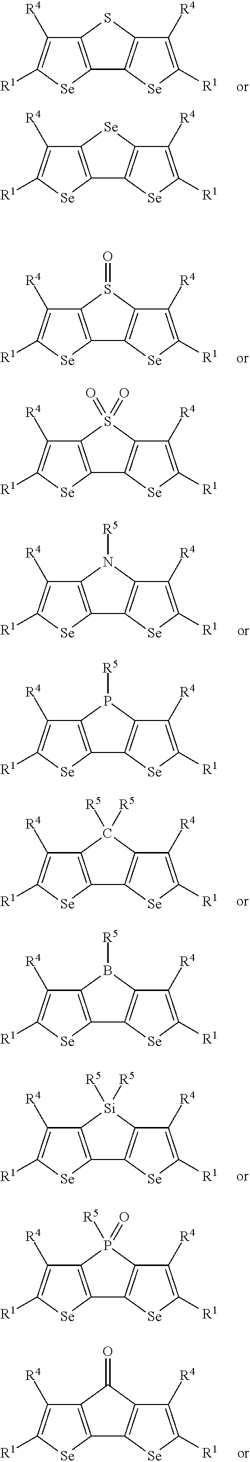 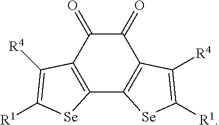
wherein R¹ is hydrogen or a halide, or a C₁-C₃₀ organic radical selected from optionally substituted alkyl, alkynyl, aryl, and heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms, R⁴ is hydrogen or optionally a C₁-C₁₈ alkyl group, and R⁵ is a C₁-C₅₀ organic radical selected from alkyl, aryl, heteroaryl.

28. The method of claim 20 wherein the fused tricyclic compound has the structure 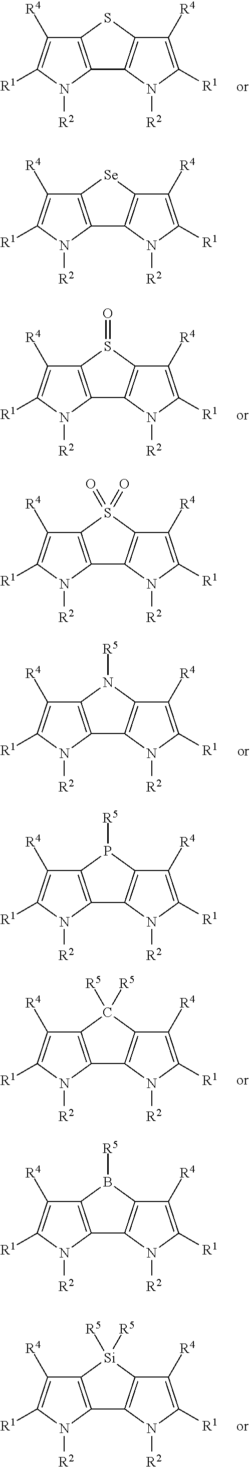 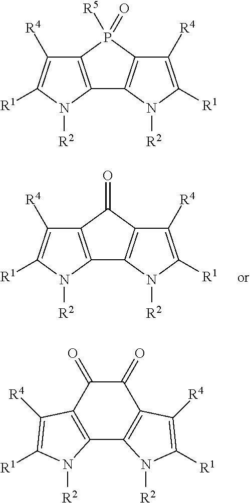
wherein R¹ is hydrogen or a halide, or a C₁-C₃₀ organic radical selected from alkyl, aryl, or heteroaryl, or —Sn(R²)₃, —Si(R²)₃, Si(OR²)₃ or —B(—OR²¹)₂ wherein each R² is an independently selected alkyl, perfluoroalkyl, or aryl and each R²¹ is an independently selected alkyl or aryl, or the R²¹ groups together form an optionally substituted alkylene group to form a ring bridging the oxygen atoms, R⁴ is hydrogen or optionally a C₁-C₁₈ alkyl group, and R⁵ is a C₁-C₅₀ organic radical selected from alkyl, aryl, heteroaryl.

29. The method of claim 20 wherein the fused tricyclic compound has the structure 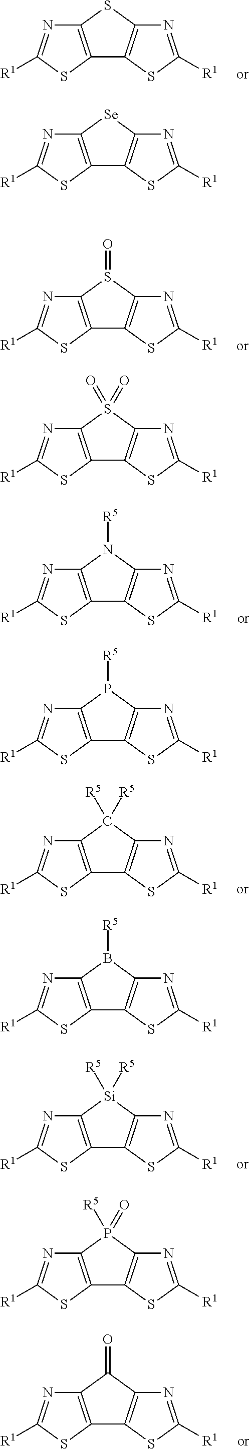 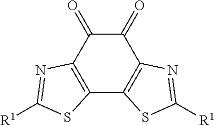
wherein R¹ is hydrogen or a halide, or a C₁-C₃₀ organic radical selected from alkyl, aryl, or heteroaryl, or —Sn(R²)₃, —Si(R²)₃, Si(OR²)₃ or —B(—OR²¹)₂ wherein each R² is an independently selected alkyl, perfluoroalkyl, or aryl and each R²¹ is an independently selected alkyl or aryl, or the R²¹ groups together form an optionally substituted alkylene group to form a ring bridging the oxygen atoms, R⁴ is hydrogen or optionally a C₁-C₁₈ alkyl group, and R⁵ is a C₁-C₅₀ organic radical selected from alkyl, aryl, heteroaryl.

30. The method of claim 29 wherein R¹ is 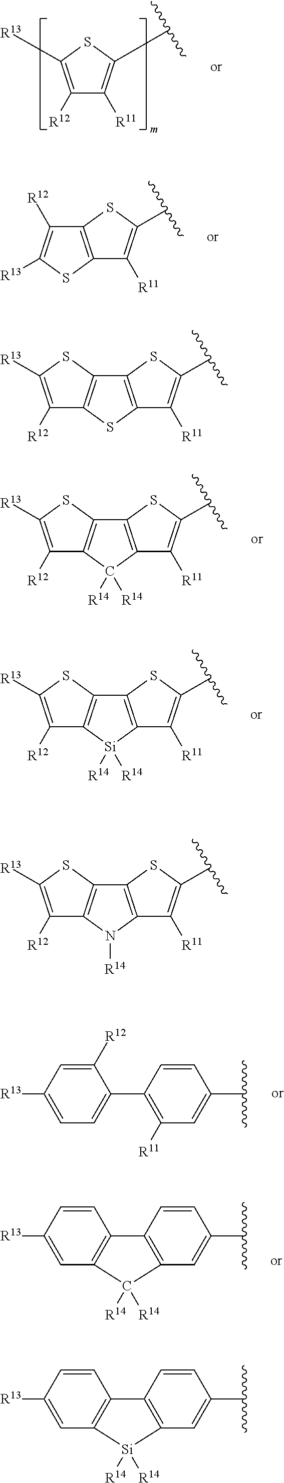 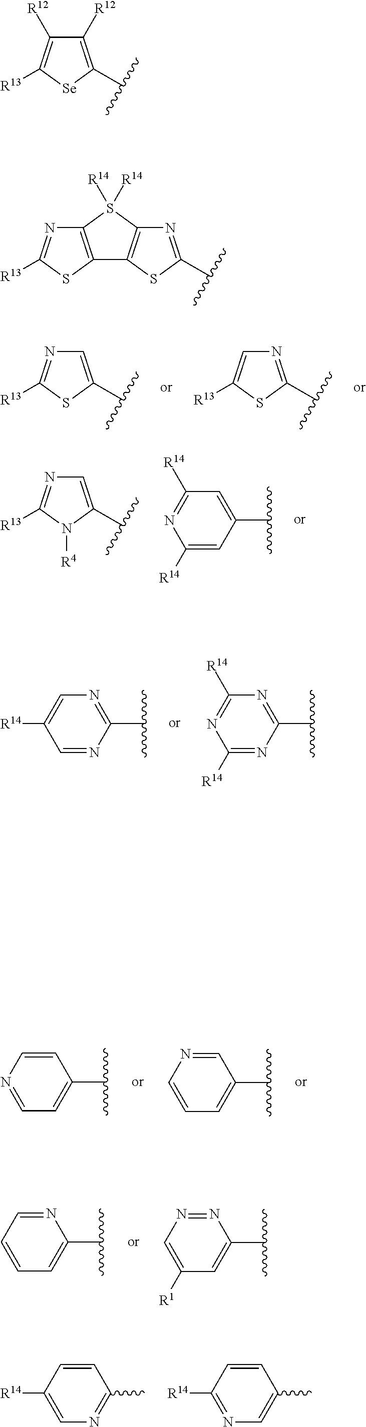 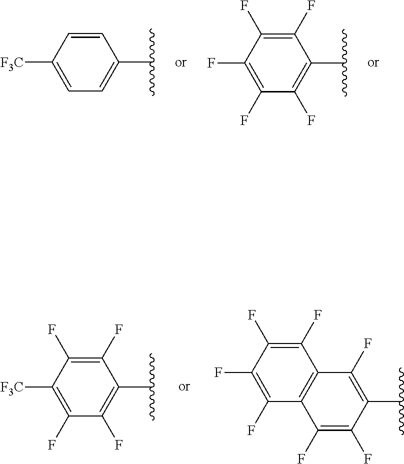
wherein m is 1, 2, 3, or 4, and R¹¹, R¹², R¹⁴ are a C₁-C₁₈ alkyl, perfluoroalkyl, alkoxy, or perfluoroalkoxy group, and R¹³ is hydrogen, —B(—OR²¹)₂, Si(R²)₃, Si(OR²)₃, or Sn(R²)₃, wherein each R² is an independently selected alkyl or aryl, and each R²¹ is an independently selected alkyl or aryl, or the R²¹ groups together form an optionally substituted alkylene group bridging the oxygen atoms.

31. A compound produced by any one of the processes of claims 1-30.

32. A composition comprising one or more of the compounds of claim 31.

33. An electronic device comprising one or more of the compounds of claim 32.

34. A compound having the structure: 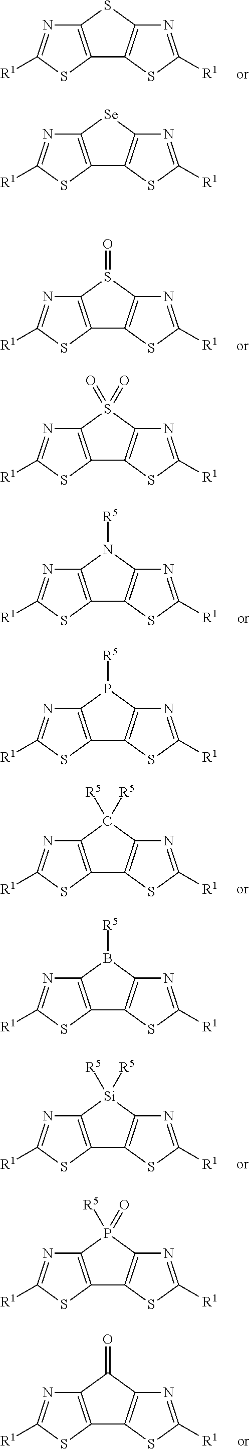 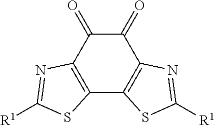
wherein R¹ is hydrogen or a halide, or a C₁-C₃₀ organic radical selected from optionally substitute alkyl, alkynyl, aryl, and heteroaryl, or —Sn(R²)₃, —Si(R²)₃, Si(OR²)₃ or —B(—OR²¹)₂ wherein each R² is an independently selected alkyl or aryl and each R²¹ is an independently selected alkyl or aryl, or the R²¹ groups together form an optionally substituted alkylene group bridging the oxygen atoms, and R⁵ is a C₁-C₅₀ organic radical selected from alkyl, aryl, heteroaryl.

35. The compound of claim 34 wherein R¹ is 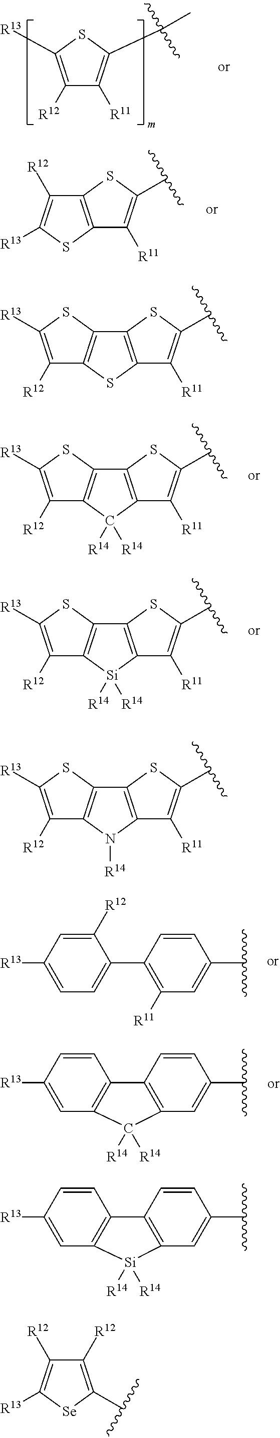 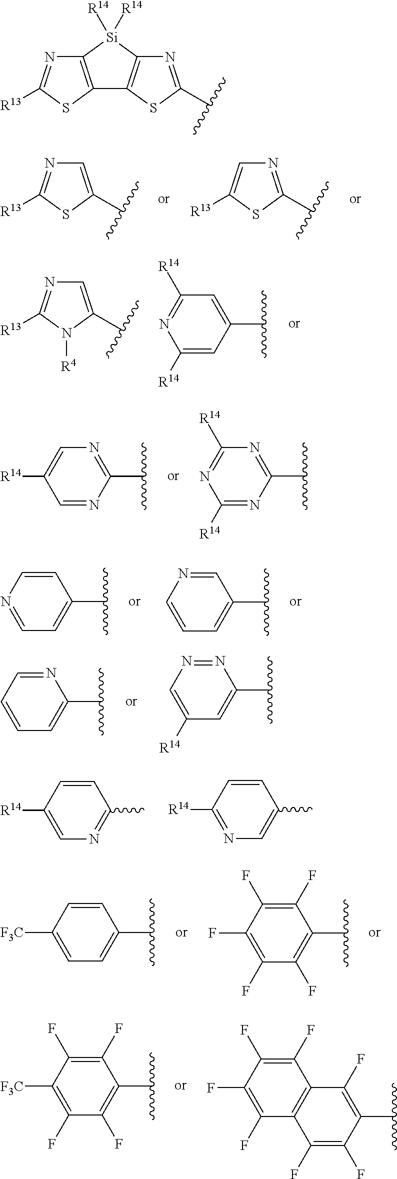
wherein m is 1, 2, 3, or 4, and R⁴, R¹¹, R¹², R¹⁴ are an independently selected C₁-C₁₈ alkyl, perfluoroalkyl, alkoxy, or perfluoroalkoxy group, and R¹³ is hydrogen, —B(—OR²¹)₂, Si(R²)₃, Si(OR²)₃ or Sn(R²)₃, wherein each R² is an independently selected alkyl or aryl, and each R²¹ is an independently selected alkyl or aryl, or the R²¹ groups together form an optionally substituted alkylene group bridging the oxygen atoms.

36. A fused tricyclic compound comprising the structure 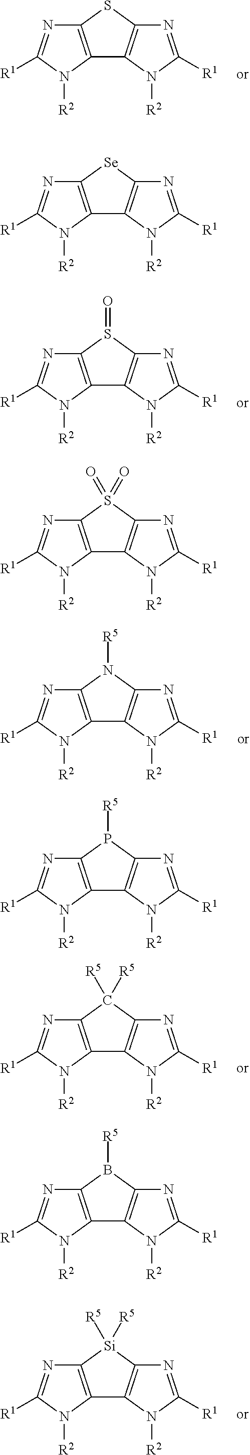 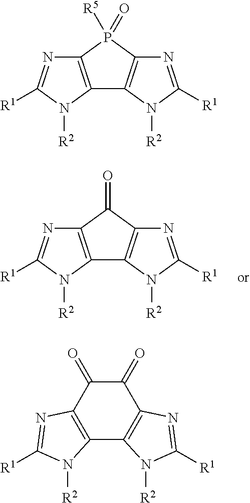
wherein R¹ is hydrogen or a halide, or a C₁-C₃₀ organic radical selected from alkyl, alkynyl, aryl, heteroaryl, or —Sn(R²)₃, —Si(R²)₃, or —B(—OR²¹)₂ wherein each R² is an independently selected alkyl or aryl and each R²¹ is an independently selected alkyl or aryl, or the R²¹ groups together form an optionally substituted alkylene group to form a ring bridging the oxygen atoms, and R⁵ is a C₁-C₅₀ organic radical selected from alkyl, aryl, heteroaryl.

37. The compound of claim 36 wherein R¹ is 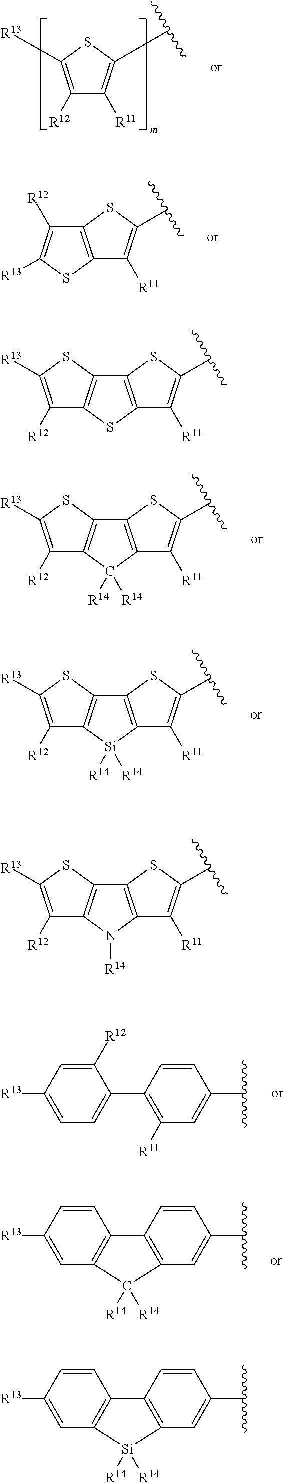 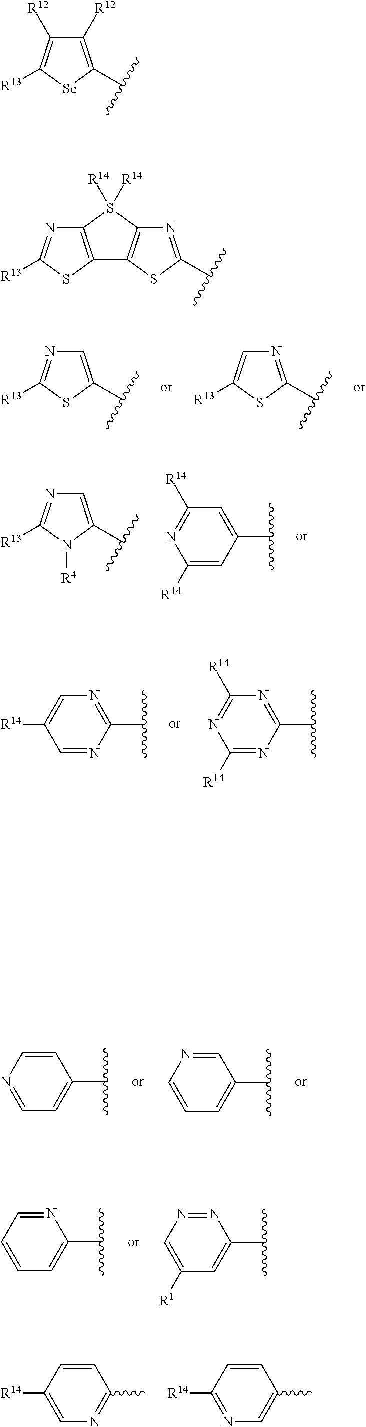 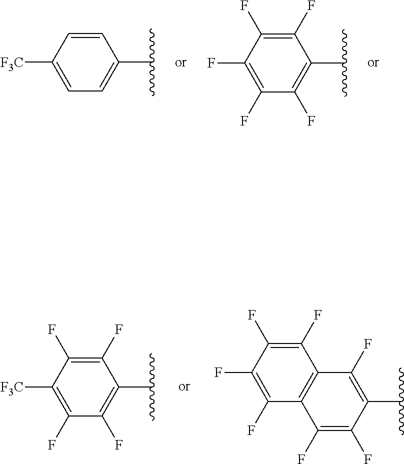
wherein m is 1, 2, 3, or 4, and R¹, R¹², R¹⁴ are a C₁-C₁₈ alkyl or alkoxy group, and R¹³ is hydrogen, halide, Si(R²)₃, or Sn(R²)₃.

38. A compound having the structure 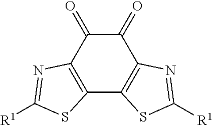
wherein R¹ is hydrogen, a halide, an optionally substituted C₁-C₃₀ alkynyl, aryl or heteroaryl, Si(R²)₃, Sn(R²)₃, or B(OR²)₂ wherein each R² is an independently selected C₁-C₁₈ alkyl or aryl, or the R² groups together form a cyclic alkylene.

39. The compound of claim 38 wherein R¹ is 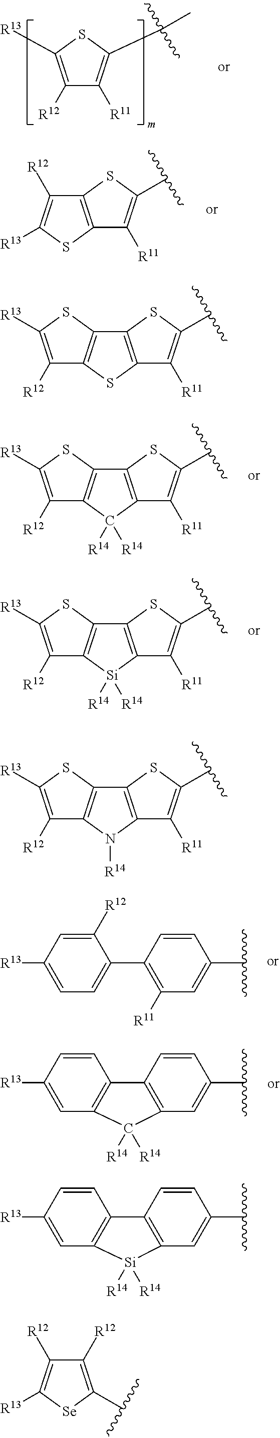 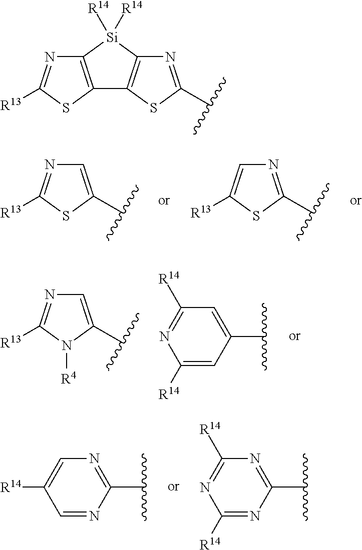
wherein m is 1, 2, 3, or 4, and R⁴, R¹, R¹², R¹⁴ are a C₁-C₁₈ alkyl, perfluoroalkyl, or alkoxy group, and R¹³ is hydrogen, halide, Si(R²)₃, or Sn(R²)₃.

40. The compound of claim 39 wherein R¹ is 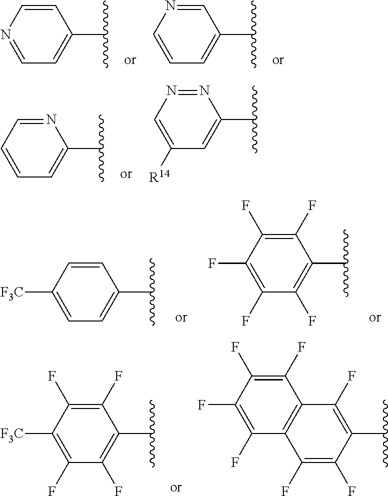
wherein R¹⁴ is hydrogen or a C₁-C₁₈ alkyl, perfluoroalkyl, or alkoxy group.

41. A compound having the structure: 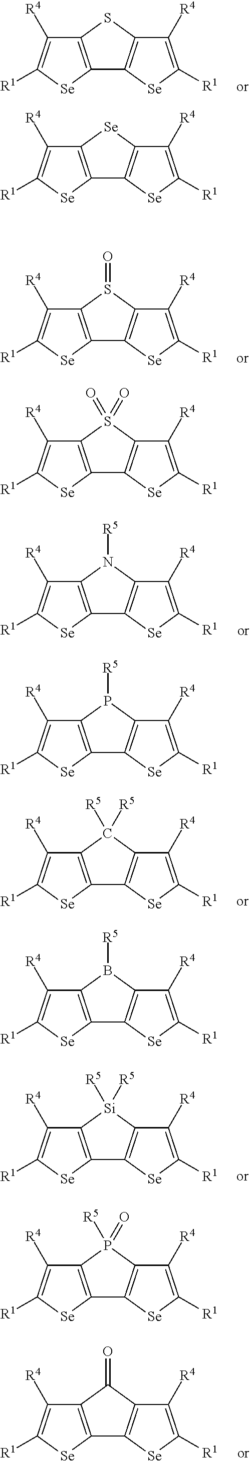 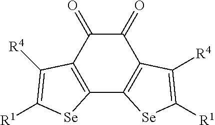
wherein R¹ is hydrogen or a halide, or a C₁-C₃₀ organic radical selected from optionally substitute alkyl, alkynyl, aryl, and heteroaryl, or —Sn(R²)₃, —Si(R²)₃, Si(OR²)₃ or —B(—OR²¹)₂ wherein each R² is an independently selected alkyl or aryl and each R²¹ is an independently selected alkyl or aryl, or the R²¹ groups together form an optionally substituted alkylene group bridging the oxygen atoms, and R⁵ is a C₁-C₅₀ organic radical selected from alkyl, aryl, heteroaryl.

42. The compound of claim 41 wherein R¹ is 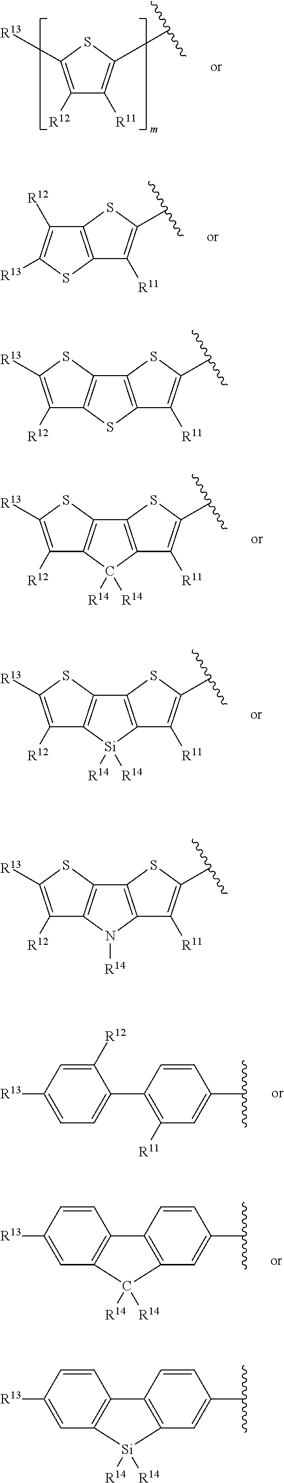 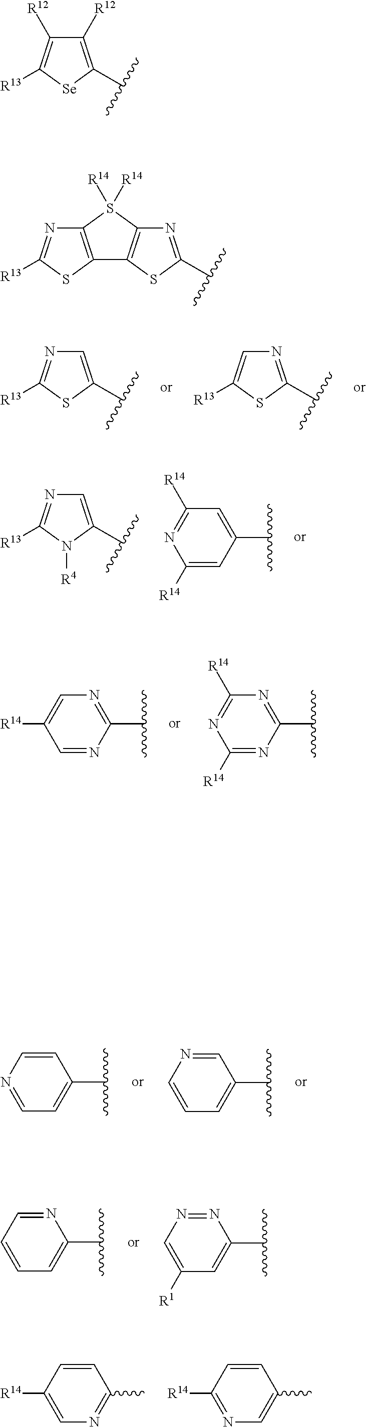 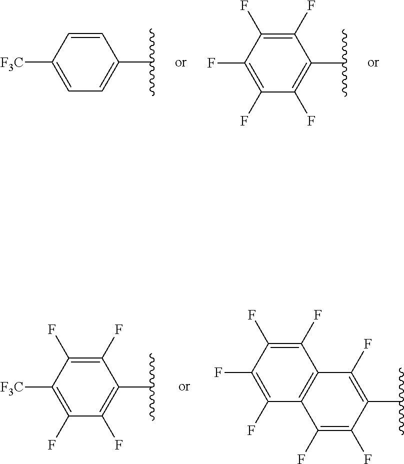
wherein m is 1, 2, 3, or 4, and R⁴, R¹¹, R¹², R¹⁴ are an independently selected C₁-C₁₈ alkyl, perfluoroalkyl, alkoxy, or perfluoroalkoxy group, and R¹³ is hydrogen, —B(—OR²¹)₂, Si(R²)₃, Si(OR²)₃ or Sn(R²)₃, wherein each R² is an independently selected alkyl or aryl, and each R²¹ is an independently selected alkyl or aryl, or the R²¹ groups together form an optionally substituted alkylene group bridging the oxygen atoms.

43. A compound having the structure 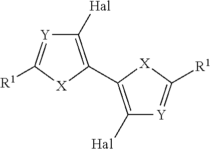
wherein
a) R¹ comprises an optionally substituted C₁-C₃₀ aryl or heteroaryl,
b) X is O, Se, or NR³ wherein R³ is a C₁-C₁₈ alkyl, fluoro alkyl, aryl, or heteroaryl, and
c) Y is CH, CR⁴, or N, wherein R⁴ is an optionally substituted C₁-C₁₈ alkyl, aryl, or heteroaryl.

44. A fused tricyclic compound having the structure 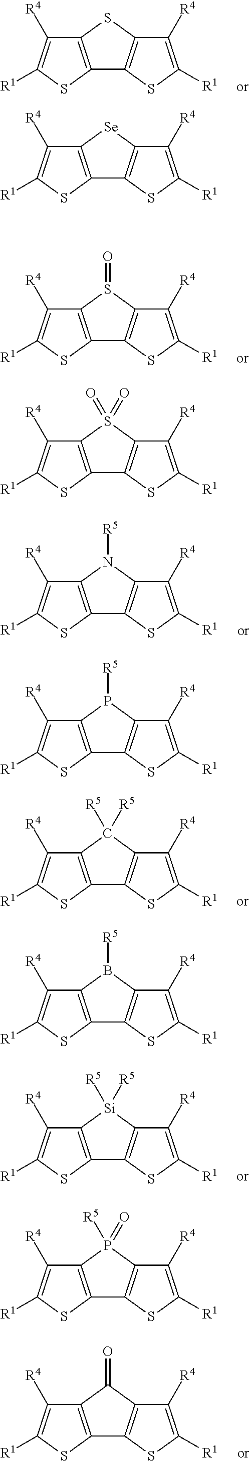 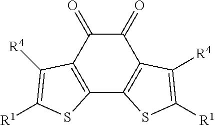
wherein R¹ is hydrogen or a halide, or a C₁-C₃₀ organic radical, R⁴ is hydrogen or optionally a C₁-C₁₈ alkyl group, and R⁵ is a C₁-C₅₀ organic radical selected from alkyl, aryl, heteroaryl.

45. The compounds of claim 44 wherein R¹ is hydrogen or a halide, or a C₁-C₃₀ organic radical selected from optionally substituted alkyl, alkynyl, aryl, and heteroaryl, or —Sn(R²)³, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms.

46. The compound of claim 44 wherein R¹ is an organic acyl compound having the formula 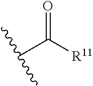
wherein R¹¹ is an aryl or heteroaryl optionally substituted with 1-10 independently selected halide, cyano, alkyl, perfluoroalkyl, acyl, alkoxy, or perfluoroalkoxy groups.

47. The compound of claim 44 wherein R¹ is 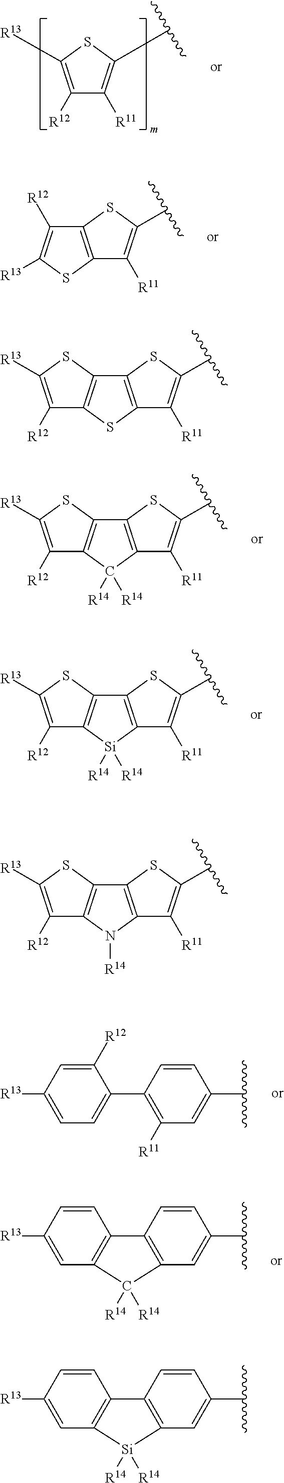 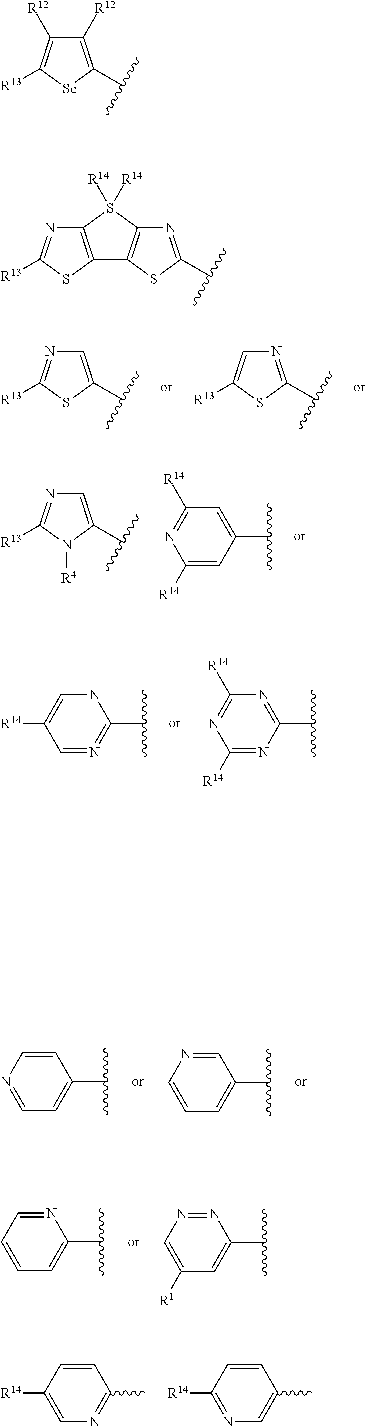 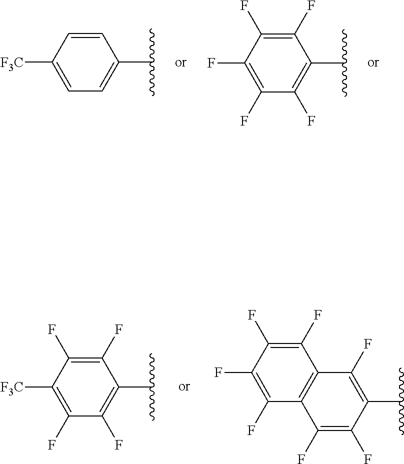
wherein m is 1, 2, 3, or 4, and R¹¹, R¹², R¹⁴ are a C₁-C₁₈ alkyl, perfluoroalkyl, alkoxy, or perfluoroalkoxy group, and R¹³ is hydrogen, —B(—OR²¹)₂, Si(R²)₃, Si(OR²)₃, or Sn(R²)₃, wherein each R² is an independently selected alkyl or aryl, and each R²¹ is an independently selected alkyl or aryl, or the R²¹ groups together form an optionally substituted alkylene group bridging the oxygen atoms.

48. The compound of claim 44 wherein R¹ is 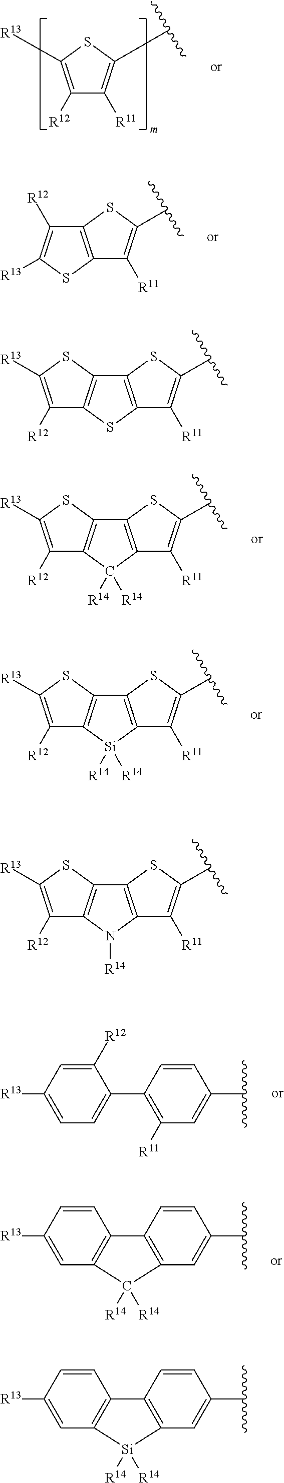 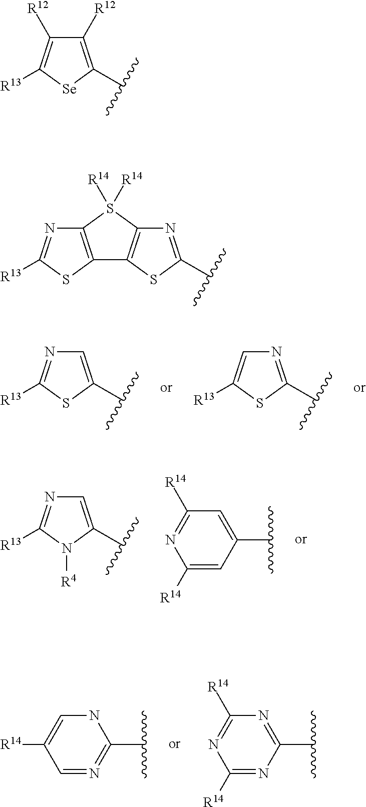
wherein m is 1, 2, 3, or 4, and R¹, R¹², R¹⁴ are a C₁-C₁₈ alkyl or alkoxy group, and R¹³ is hydrogen, —B(—OR²¹)₂, Si(R²)₃, or Sn(R²)₃.

49. The compound of claim 44 wherein R¹ is

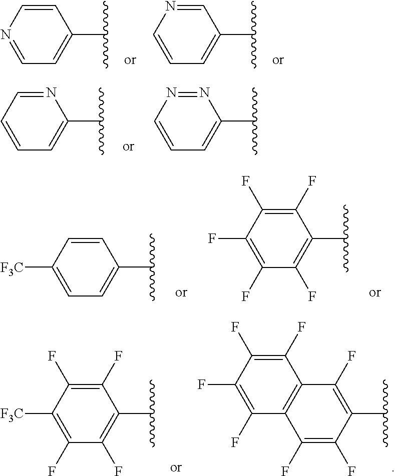

50. A compound having the structure 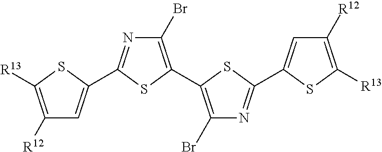
wherein R¹² is a C₁-C₁₈ alkyl or alkoxy group and R¹³ is hydrogen, halide, Si(R²)₃, wherein each R² is an independently selected alkyl or aryl.

51. A polymer or copolymer comprising a repeat unit having the structure 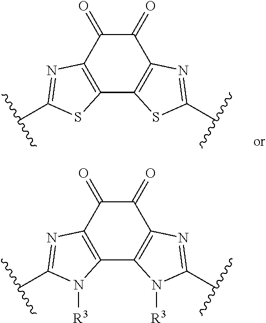
wherein R³ is a C₁-C₁₈ alkyl, perfluoroalkyl, aryl, or heteroaryl.

52. A polymer or copolymer comprising a repeat unit having the structure 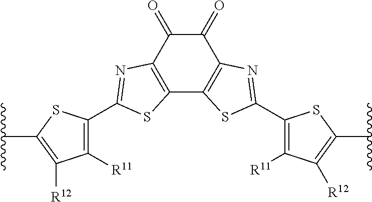
wherein R¹¹ and R¹² are hydrogen or a C₁-C₁₈ alkyl.

53. A mono or bis ketal compound having the formula 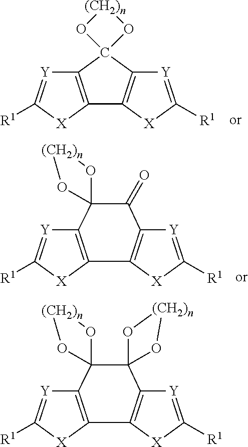
wherein
a) wherein R¹ is hydrogen or a halide, or a C₁-C₃₀ organic radical;
b) X is O, S, Se, or NR³ wherein R³ is a C₁-C₁₈ alkyl, perfluoroalkyl, aryl, or heteroaryl; and
c) Y is CH, CR⁴, or N, wherein R⁴ is a C₁-C₁₈ alkyl, aryl, or heteroaryl.

54. The compound of claim 54, wherein the C₁-C₃₀ organic radical is selected from optionally substituted alkyl, alkynyl, aryl, and heteroaryl, or —Sn(R²)₃, —Si(R²)₃, Si(OR²)₃ or —B(—OR²¹)₂ wherein each R² is an independently selected alkyl or aryl, and each R²¹ is an independently selected alkyl or aryl, or the R²¹ groups together form an optionally substituted alkylene group bridging the oxygen atoms.
